(19) United States
(12) Patent Application Publication
(10) Pub. No.: US 2011/0263540 A1
(43) Pub. Date: Oct. 27, 2011

(54) SMALL-MOLECULE INHIBITORS OF PROTEIN SYNTHESIS INACTIVATING TOXINS

(76) Inventors: Yuan-Ping Pang, Rochester, MN (US); Nilgun Ereken Tumer, Belle Mead, NJ (US); Charles B. Millard, Frederick, MD (US)

(21) Appl. No.: 13/055,813

(22) PCT Filed: Jul. 24, 2009
(86) PCT No.: PCT/US09/51683

§ 371 Date: May 3, 2011

Related U.S. Application Data
(60) Provisional application No. 61/083,667, filed on Jul. 25, 2008.

(51) Int. Cl. A61K 31/655; A61K 31/47; A61K 31/44; A61K 31/4025; A61K 31/402; G06G 7/60; A61K 31/454; A61K 31/381; A61K 31/4015; A61K 31/404; A61K 31/4035; A61P 39/02; A61K 31/196; A61K 31/5377

(52) U.S. Cl. 514/150; 514/563; 514/311; 514/355; 514/422; 514/425; 514/235.5; 514/326; 514/443; 514/565; 514/423; 514/418; 514/567; 514/417; 703/11

(57) ABSTRACT

Small-molecule inhibitors of a protein synthesis inhibiting toxin, e.g., ricin, abrin, Shiga, and Shiga-like toxins, as well as methods of using the inhibitors are provided. Further provided are methods of identifying small-molecule inhibitors of a protein synthesis inhibiting toxin.

### CROSS-REFERENCE TO RELATED APPLICATIONS

This application claims priority to U.S. Provisional Application Ser. No. 61/083,667, filed on Jul. 25, 2008, which is incorporated by reference in its entirely herein.

### STATEMENT AS TO FEDERALLY FUNDED RESEARCH

Studies described herein were supported by the U.S. Army Medical Research Acquisition Activity (W81XWH-04-2-0001) and the NIH/NIAID (1U01AI082120-01). The Government has certain rights in this invention.

### TECHNICAL FIELD

This disclosure relates to materials and methods for inhibiting a protein synthesis inactivating toxin. In particular, provided herein are materials and methods for inhibiting ricin, Shiga toxins, Shiga-like toxins (e.g., E. coli 0157:57), and abrin toxins.

### BACKGROUND

Ricin is a potent heterodimeric cytotoxin isolated from the seeds of the castor plant, Ricinus communis, Euphorbiacea. The protein consists of a lectin B chain (RTB), which can bind cell surfaces and is linked by disulfide bonds to an A chain (RTA), which enzymatically depurinates a specific adenine residue in 28S rRNA. Ricin is an extraordinarily toxic molecule that attacks ribosomes, thereby inhibiting protein synthesis. As RTA exhibits this type of destructive catalytic activity, RTA is commonly referred to as a type II ribosome inactivating protein (RIP).

The toxic consequences of ricin are due to the biological activity of RTA. RTB binds the toxin to cell surface receptors and then RTA is transferred inside the cell where inhibition of ribosome activity occurs. Ricin has an LD₅₀ of approximately 1 μg/kg body weight for mice, rats, and dogs. The toxic dose for humans is likely to be in the μg/kg range which ranks it among the most toxic substances known.

Shiga toxins are a family of related toxins with two major groups, Stx1 and Stx2, whose genes are considered to be part of the genome of lambdoid prophages. The most common sources for Shiga toxin are the bacteria S. dysenteriae and the Shigatoxigenic group of Escherichia coli (STEC), which includes serotype O157:H7 and other enterohemorrhagic E. coli. Shiga toxins act to inhibit protein synthesis within target cells by a mechanism similar to that of ricin toxin.

Abrin is a natural poison that is found in the seeds of a plant called the rosary pea or jequirity pea. Like ricin, abrin can penetrate cells and inhibit protein synthesis. Both ricin and abrin have potential medical use as components of immunotoxins.

Given the above, there is interest in identifying or designing potent inhibitors of ricin, Shiga, Shiga-like, and abrin toxins. These inhibitors could, in principle, be used as co-treatment to limit or control immunotoxin toxicity, or could be used as an antidote or prophylaxis to poison attacks or food poisonings.

### SUMMARY

This disclosure provides materials and methods for inhibiting a protein synthesis inactivating toxin. For example, small-molecule inhibitors of ricin, abrin, Shiga toxins, and Shiga-like toxins are provided. Methods for using such small-molecule inhibitors to treat, prevent, or ameliorate one or more symptoms of protein synthesis inactivating toxin poisoning are also provided. Kits and articles of manufacture containing one or more small-molecule inhibitors and accessory items are also provided. Further provided is a method of identifying inhibitors of protein synthesis inactivating toxins.

Provided herein are compounds according to Formula I-A:

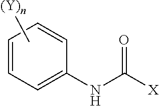

wherein:
X is selected from C₁₋₁₀ alkyl, C₅₋₁₂ cycloalkyl, C₅₋₁₂ aryl, or C₅₋₁₂ heteroaryl, wherein the alkyl, cycloalkyl, aryl, or heteroaryl may be substituted with one or more of C₁₋₁₀ alkyl, OR¹, NO₂, CONR¹R², COR¹, and halo;
each Y is independently H, C₁₋₁₀ alkyl, CO₂R¹, OR¹, or halo;
R¹ and R² are independently H, C₁₋₁₀ alkyl, and aryl; and
n is 1, 2, or 3; or a pharmaceutically acceptable salt or derivative thereof.

In some embodiments, n is 2 and one Y is in the meta position on the ring and the other Y is in the para position on the ring. In some embodiments, the Y in the meta position is a C₁₋₁₀ alkyl. In some embodiments, n is 3 and one Y is in the para position on the ring and the remaining two Y moieties are in the meta positions on the ring.

In some embodiments, the compound according to Formula I-A is selected from:

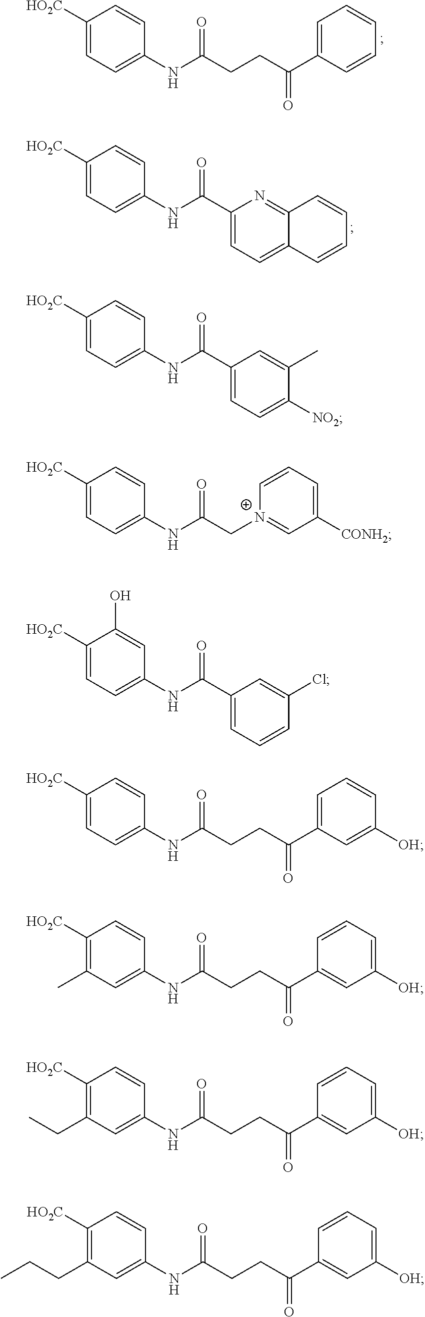

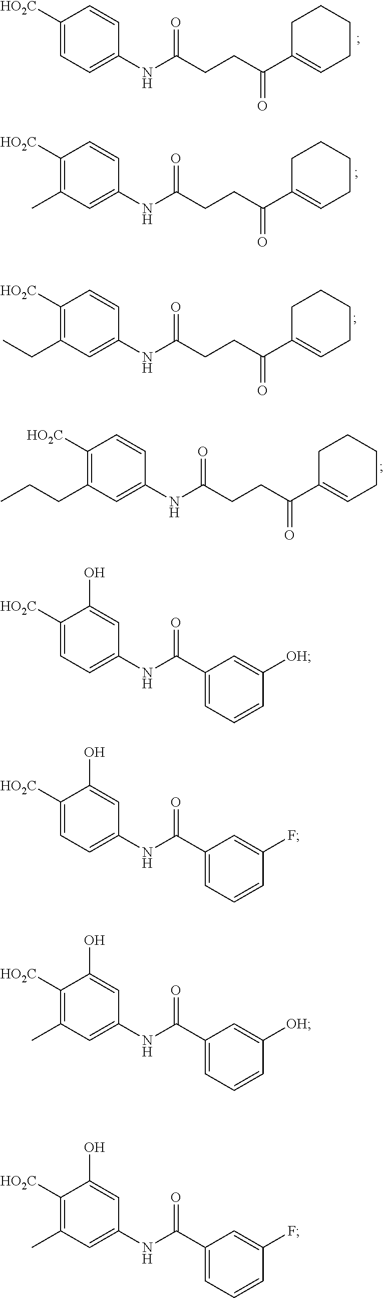

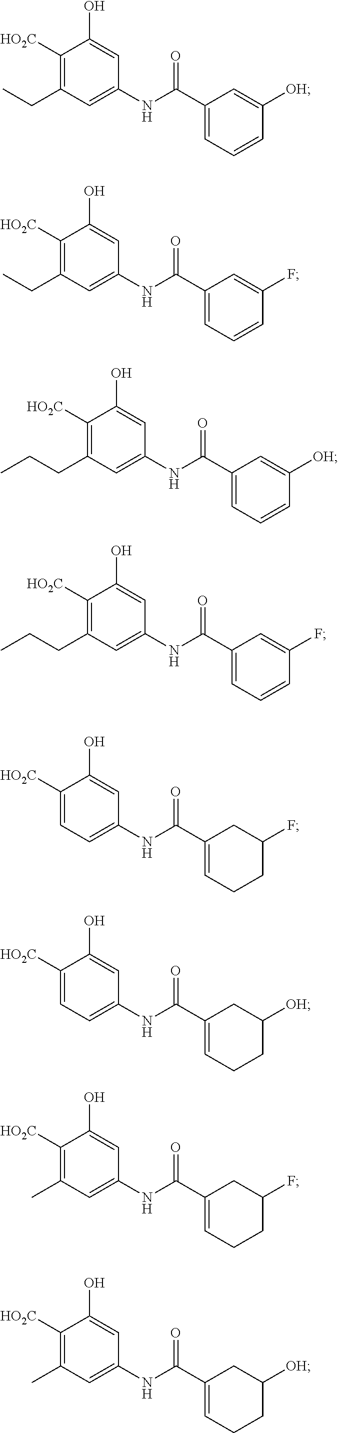

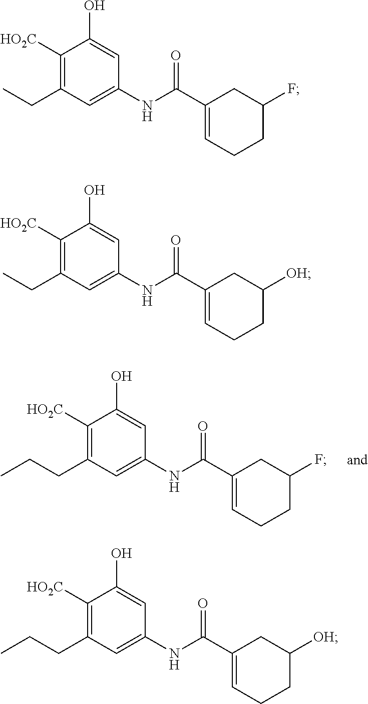

or
a pharmaceutically acceptable salt or derivative thereof.

Also provided herein are compounds according to Formula II-A:

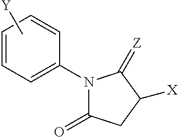

wherein:
X is selected from CO₂R¹, NR¹R², or C₅₋₁₂ heterocycloalkyl;
Y is selected from H, C₁₋₁₀ alkyl, OR¹, or halo;
Z is absent or O;
R¹ is H or C₁₋₁₀ alkyl; and
R² is selected from H, C₁₋₁₀ alkyl; and C₅₋₁₂ cycloalkyl, wherein the alkyl and cycloalkyl may be substituted with C₁₋₁₀ alkyl or C₅₋₁₂ heterocycloalkyl, wherein the heterocycloalkyl may be substituted with a C₁₋₁₀ alkyl; or a pharmaceutically acceptable salt or derivative thereof.

In some embodiments, the compound according to Formula II-A is selected from:

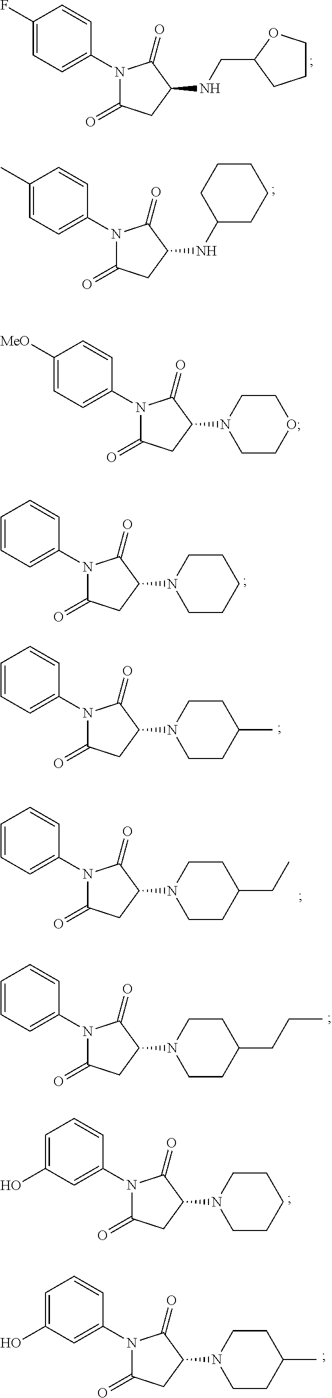

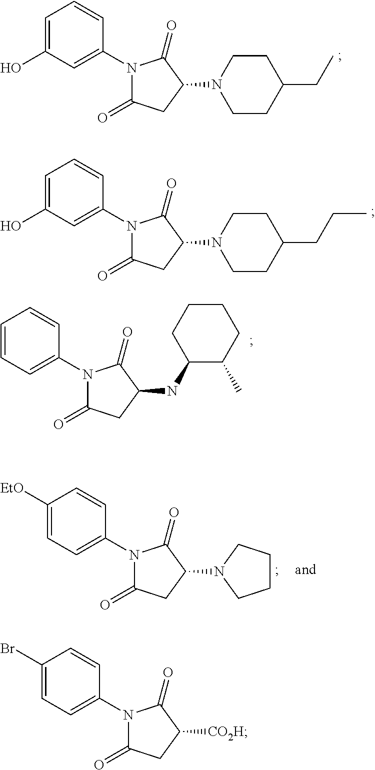

or
a pharmaceutically acceptable salt or derivative thereof.

This disclosure also provides compounds according to Formula III-A:

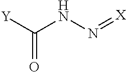

wherein:
X is C₁₋₁₀ alkyl, C₅₋₁₂ cycloalkyl, or C₅₋₁₂ heteroalkyl, wherein the alkyl and heteroaryl can be substituted with one or more of CO₂R¹, OR¹, and halo;
Y is selected from C₅₋₁₂ aryl, C₅₋₁₂ cycloalkyl, and C₅₋₁₂ heterocycle, wherein the heterocycle can be substituted with one or more of OR¹ and NR¹R²; and
R¹ and R² are independently selected from H and C₁₋₁₀ alkyl; or a pharmaceutically acceptable salt or derivative thereof.

In some embodiments, the compound according to Formula III-A is selected from:

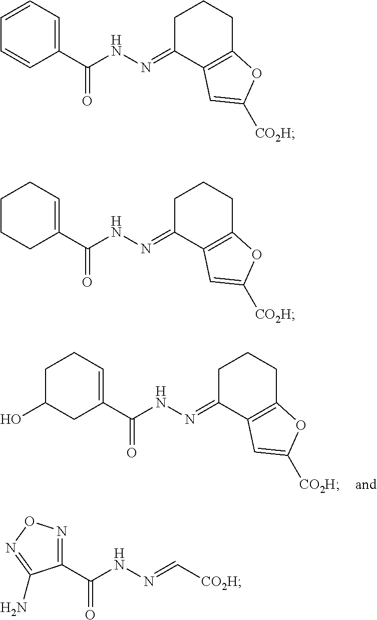

or
a pharmaceutically acceptable salt or derivative thereof.

Further provided herein is a compound according to Formula IV-A:

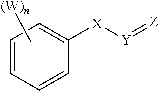

each W is independently C₁₋₁₀ alkyl, CO₂R¹, OR¹, or halo;
X is absent or NH;

### Y is N or CH;

Z is selected from C₁₋₁₀ alkyl, C₁₋₁₀ alkenyl, C₁₋₁₀ aralkyl, C₁₋₁₀ heteroaralkyl, C₅₋₁₂ cycloalkyl, and C₅₋₁₂ heterocycle, wherein the alkyl, aralkyl, heteroaralkyl, and heterocycle can be substituted with one or more of C₁₋₁₀ alkyl, C(NH)NH₂, NR¹R², (CH₂)ₘNR¹R², OR¹, (CH₂)ₘOR¹, CN, NO₂, COR¹, CO₂R¹, CF₃, OCF₃, SO₃H, halo, and ═O;
R¹ and R² are independently selected from H, COCH₃, C₁₋₁₀ alkyl, (CH₂)ₘOH, and C₁₋₁₀ aryl;
m is an integer from one to three; and
n is an integer from one to three; or a pharmaceutically acceptable salt or derivative thereof.

In some embodiments, a compound according to Formula IV-A is selected from:

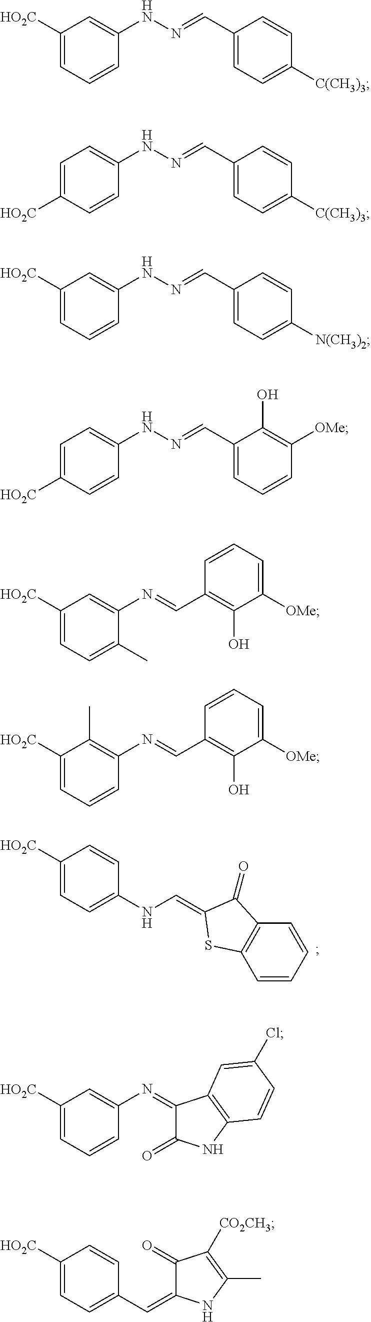

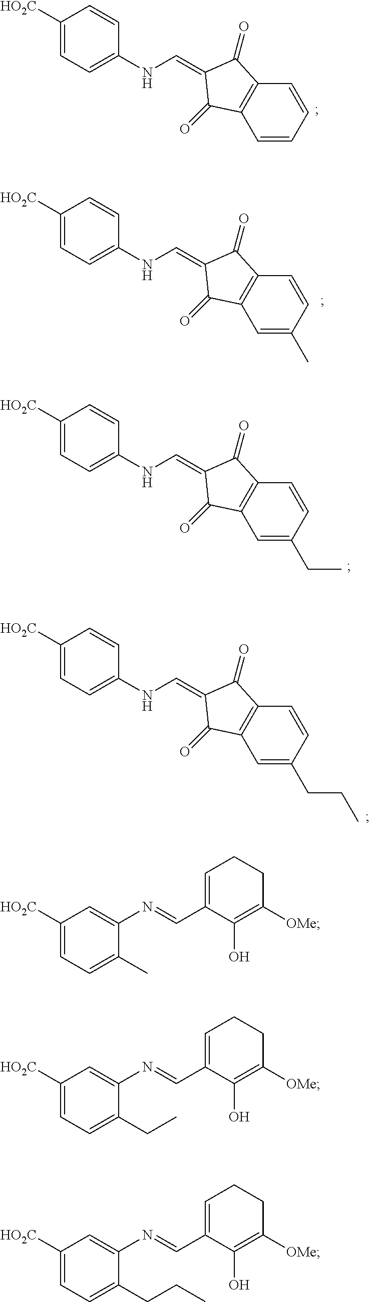

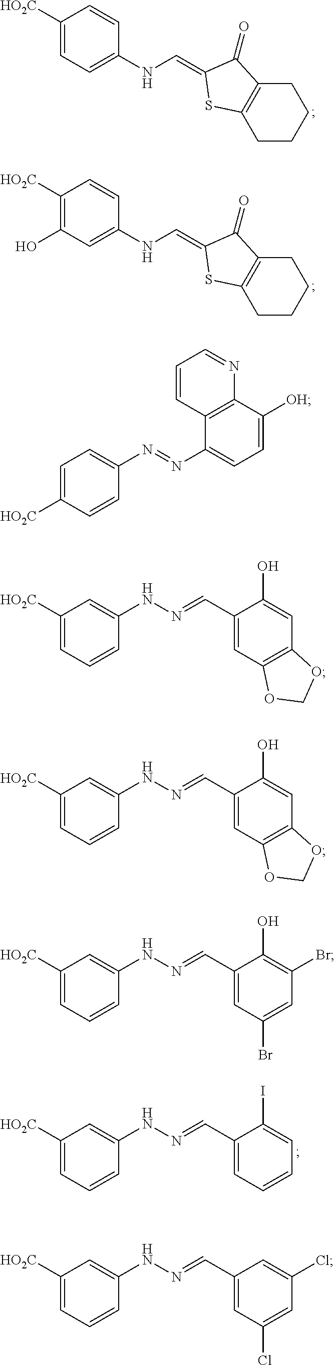

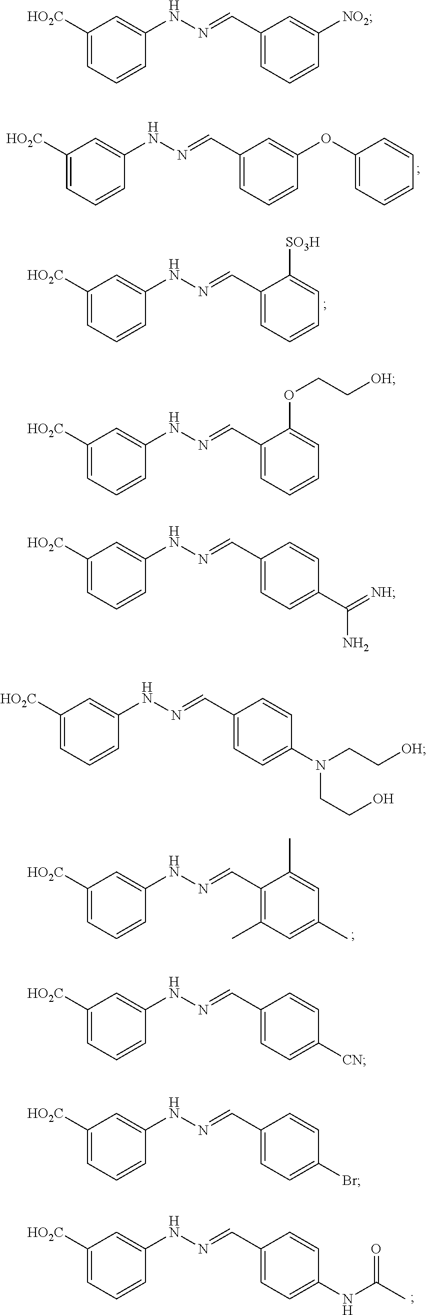

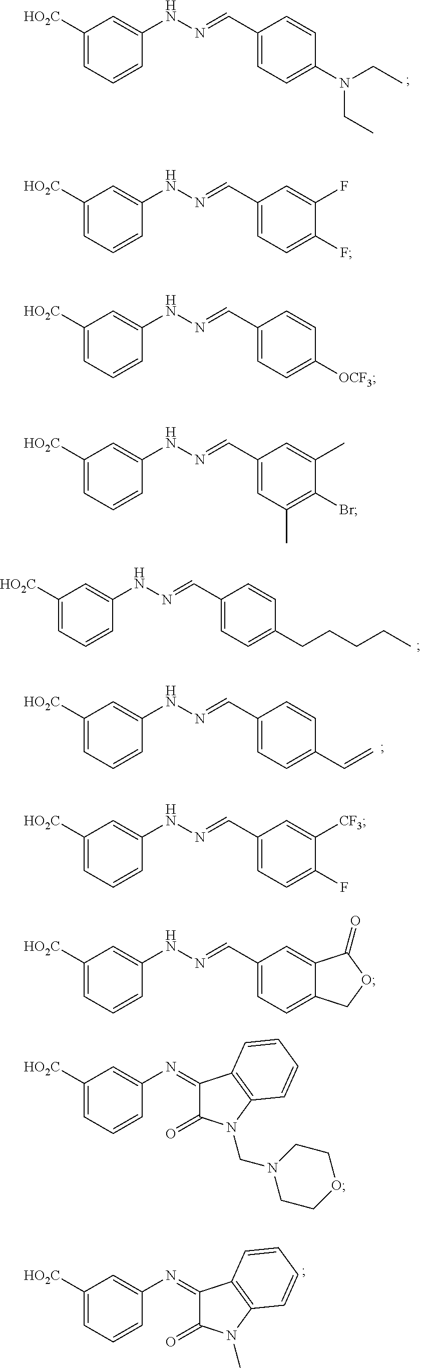

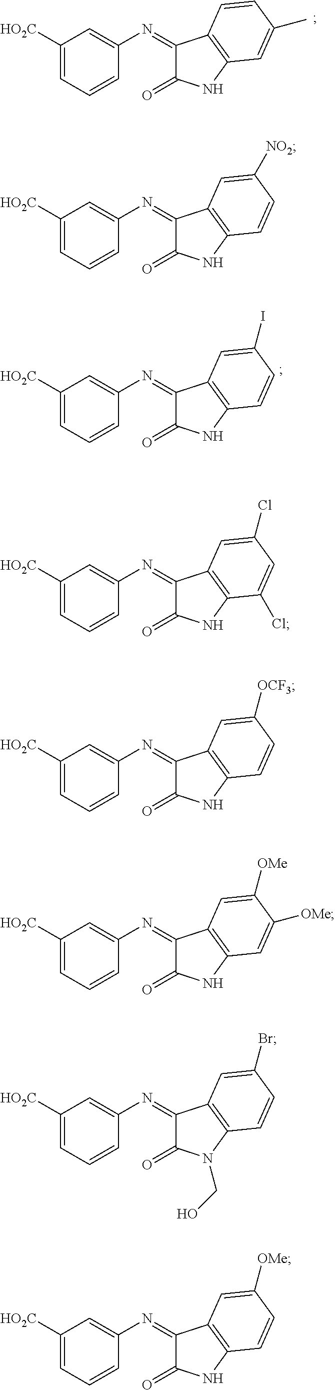

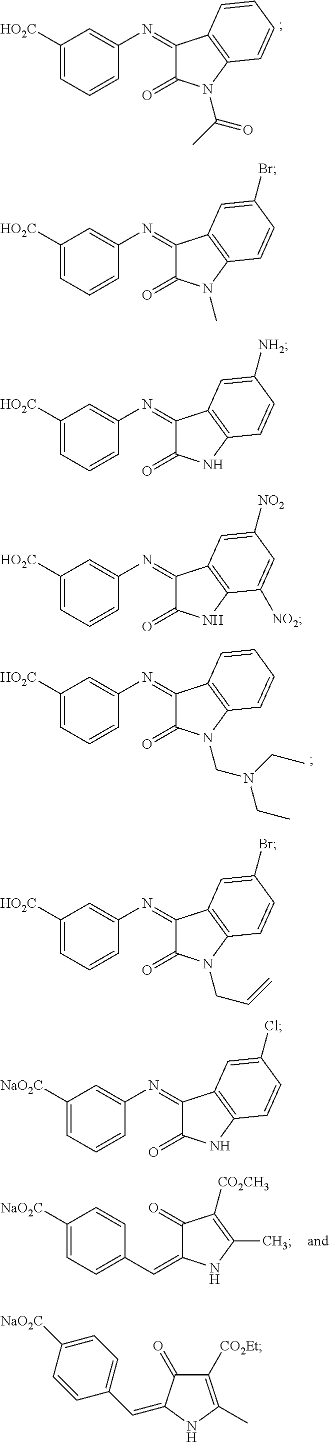

or a pharmaceutically acceptable salt or derivative thereof.

This disclosure also provides a compound according to Formula V-A:

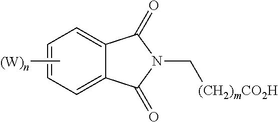

wherein
each W is independently C₁₋₁₀ alkyl, C₂₋₁₀ alkenyl, C₂₋₁₀ alkynyl, CO₂R¹, OR¹, halo, NO₂, NR¹R², or two W come together to form a fused aryl, heteroaryl, cycloalkyl, or heterocycloalkyl, wherein the alkyl, alkenyl or alkynyl can be unsubstituted or substituted with CO₂R¹, OR¹, or halo;
R¹ and R² are independently selected from H and C₁₋₁₀ alkyl;
m is an integer from zero to five; and
n is an integer from zero to three; or a pharmaceutically acceptable salt or derivative thereof.

In some embodiments, a compound according to Formula V-A is selected from:

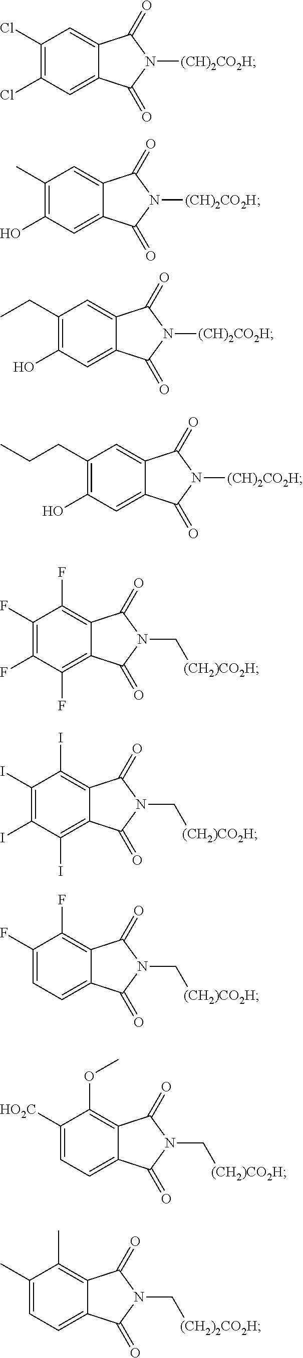

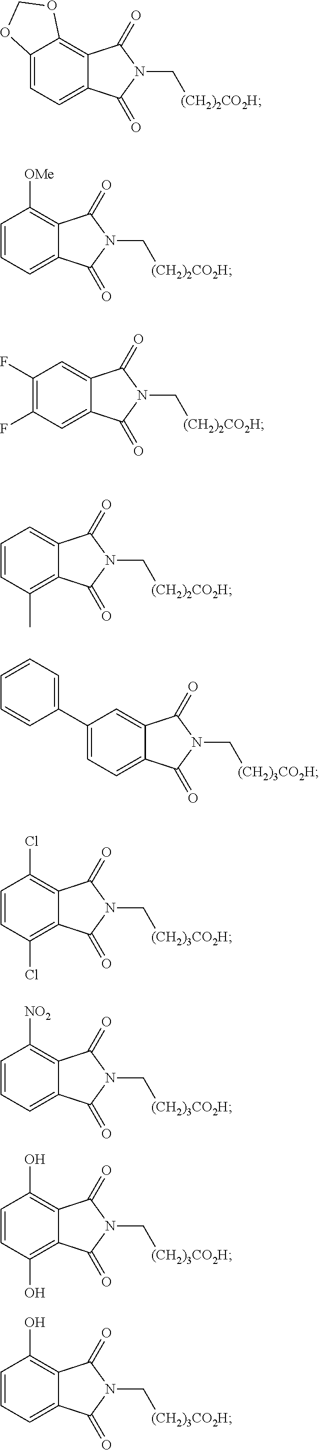

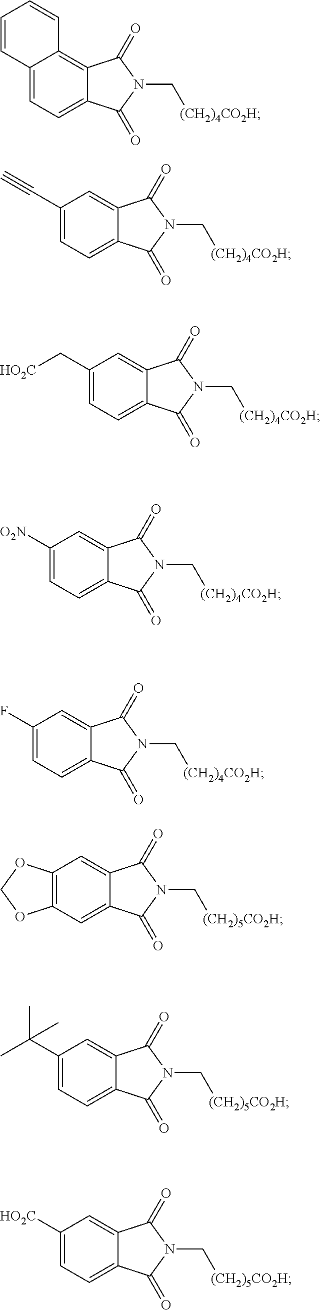

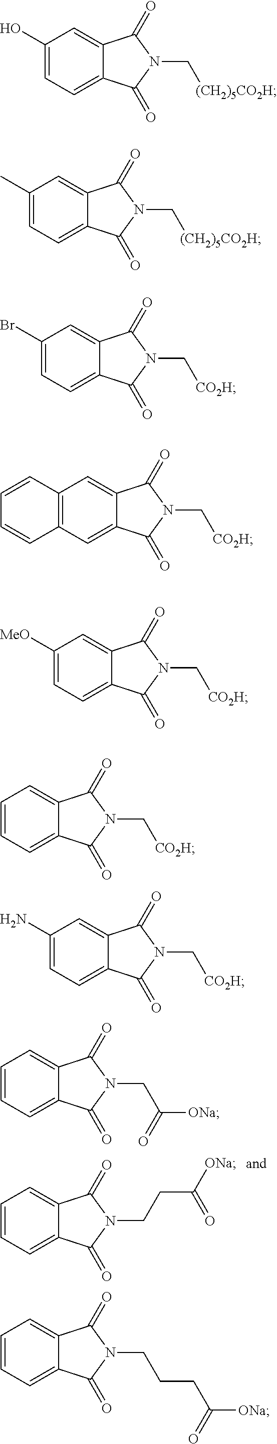

or a pharmaceutically acceptable salt or derivative thereof.

Provided herein are compounds according to Formula VI-A:

A-(CH₂)ₙ-B

wherein
A is selected from the group consisting of:

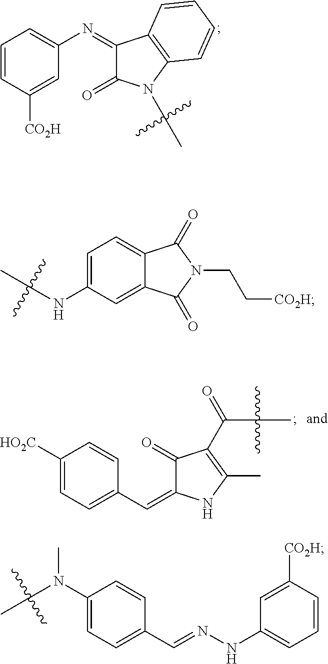

B is selected from the group consisting of:

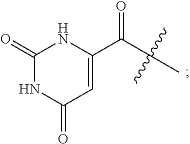

and
n is an integer from four to ten; or a pharmaceutically acceptable salt or derivative thereof.

Further provided herein are compounds according to formula VI-A is selected from:

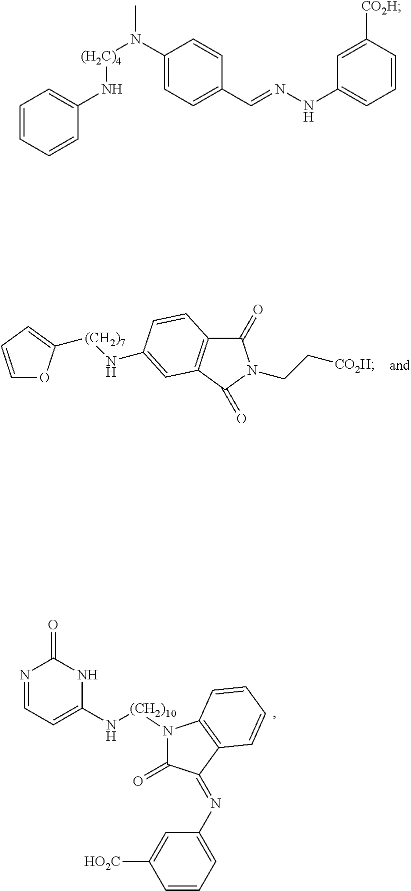

or a pharmaceutically acceptable salt or derivative thereof.

Further provided are compounds selected from:

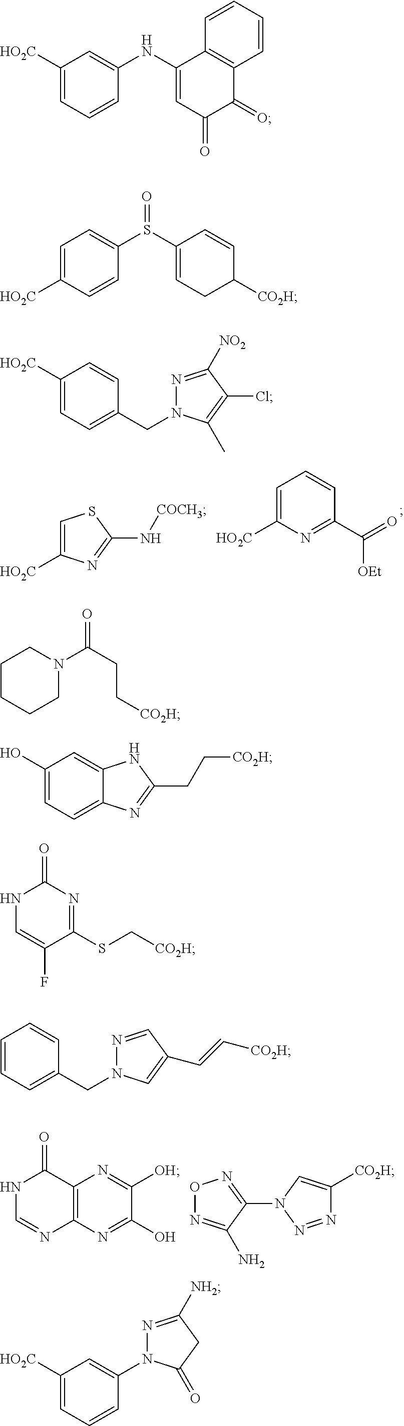

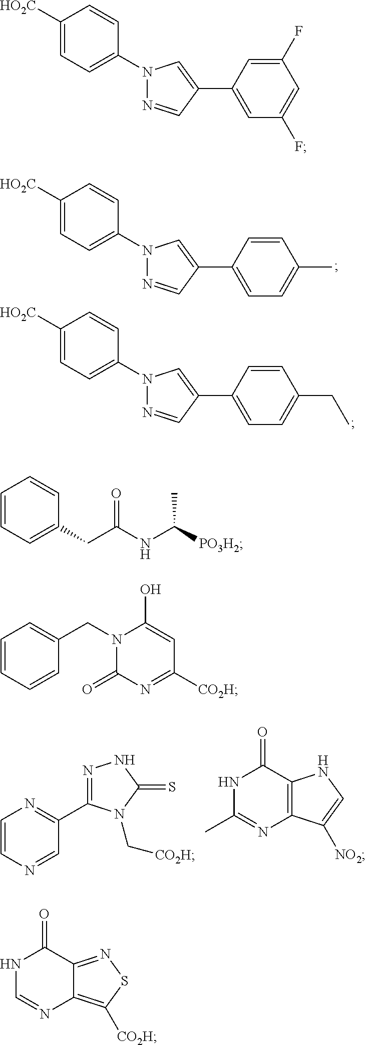

or a pharmaceutically acceptable salt or derivative thereof.

Provided herein is a method of treating or ameliorating one or more symptoms associated with a protein synthesis inactivating toxin poisoning in a subject, the method comprising administering to the subject one or more of the compounds described herein, or a pharmaceutically acceptable salt or derivative thereof.

In some embodiments, the subject is a human.

In some embodiments, the protein synthesis inactivating toxin is selected from: a ribonuclease, an N-glycosidase, and an ADP-ribosyltransferase. In some embodiments, the N-glycosidase is selected from a Type I ribosome inhibiting protein or a Type II ribosome inhibiting protein. In some embodiments, the Type II ribosome inhibiting protein is ricin (e.g., the ricin A chain). In some embodiments, the ribosome inhibiting protein is Stx2 (e.g., subunit A). In some embodiments, the protein synthesis inactivating toxin is ricin, abrin, a Shiga toxin, or a Shiga-like toxin.

Further provided herein is a method of treating or ameliorating one or more symptoms associated with ricin, abrin, a Shiga toxin, or a Shiga-like toxin poisoning in a subject, the method comprising administering to the subject one or more of the compounds described herein, or a pharmaceutically acceptable salt or derivative thereof. In some embodiments, the ricin is a heterodimeric ricin. In some embodiments, the ricin comprises the ricin A chain. In some embodiments, the ricin is the ricin A chain. In some embodiments, the Shiga-like toxin is Stx2. In some embodiments, the Shiga-like toxin comprises subunit A of Stx2. In some embodiments, the Shiga-like toxin is Stx2 subunit A.

Further provided herein is a pharmaceutical composition comprising a compound as described herein and a pharmaceutically acceptable carrier, excipient, or adjuvant.

This disclosure also provides a method of inhibiting type II ribosome inactivating protein poisoning in a subject, the method comprising administering to the subject one or more of the compounds described herein in combination with a type II ribosome inactivating protein vaccine.

Further provided is a method of reducing incapacitating local tissue damage at the portal of a type II ribosome inactivating protein entry in a subject, the method comprising administering to the subject one or more of the compounds described herein in combination with a type II ribosome inactivating protein vaccine. Also provided is a method of reducing incapacitating lung damage in a subject, the method comprising administering to the subject one or more of the compounds described herein in combination with a type II ribosome inactivating protein vaccine.

A computer-assisted method of generating a test inhibitor of the active site of ricin is also provided, the method using a programmed computer comprising a processor and an input device, the method comprising:

(a) inputting on the input device data comprising a docking box surrounded by one or more amino acid residues of the active site of ricin, the residues having a confirmation as set forth in crystal structure PDB code 1IFS;

(b) docking into the docking box a test inhibitor molecule using the processor; and

(c) determining, based on the docking, whether the test inhibitor molecule would be capable of interacting with one or more residues of the ricin active site.

In some embodiments, the docking box is surrounded by one or more of residues Asp100, Ile-104, Asp75, Asn78, Tyr80, Val82, Phe93, Gly120, Gly121, Asn122, His94, Pro95, and Asp96 of ricin chain A. In some embodiments, the docking box is surrounded by one or more of residues Tyr80, Val81, Phe93, Gly121, Asn122, Tyr123, Ile172, Arg180, Ala79, Ser176, Glu177, and Leu126 of ricin chain A.

In some embodiments, the test inhibitor molecule comprises one or more of a type-I molecule, a type-II molecule, or mixtures thereof. In some embodiments, the type-1 molecule is capable of interacting with one or more of residues Asp100, Ile-104, Asp75, Asn78, Tyr80, Val82, Phe93, Gly120, Gly121, Asn122, His94, Pro95, and Asp96 of ricin chain A. In some embodiments, the type-2 molecule is capable of interacting with one or more of residues Tyr80, Val81, Phe93, Gly121, Asn122, Tyr123, Ile172, Arg180, Ala79, Ser176, Glu177, and Leu126 of ricin chain A. In some embodiments, the type-1 molecule is tethered to type-2 molecule resulting in a dimer capable of interacting with one or more of residues Asp100, Ile-104, Asp75, Asn78, Tyr80, Val82, Phe93, Gly120, Gly121, Asn122, His94, Pro95, Asp96, Tyr80, Val81, Phe93, Gly121, Asn122, Tyr123, Ile172, Arg180, Ala79, Ser176, Glu177, and Leu126 of ricin chain A.

In some embodiments, the method further includes evaluating the inhibitory activity of the test inhibitor in cell-free in vitro translation assay. In some embodiments, the method further includes evaluating the inhibitory activity of the test inhibitor in a neutralization assay. In some embodiments, the method further includes evaluating the inhibitory activity of the test inhibitor in a pre-treat assay. In some embodiments, the method further comprising evaluating the inhibitory activity of the test inhibitor in a rescue assay.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 details the results for a luciferase activity assay using yeast cell-free translation of compound I-1 (50 nM) against RTA (50 nM).

FIG. 2 details the results for a luciferase activity assay using yeast cell-free translation of compound IV-5 (50 nM) against RTA (50 nM).

FIG. 3 details the results for a luciferase activity assay using yeast cell-free translation of compound IV-7 (50 nM) against RTA (50 nM).

FIG. 4 details the results for a luciferase activity assay using yeast cell-free translation of compound III-1 (50 nM) against RTA (50 nM).

FIG. 5 details the results for a luciferase activity assay using yeast cell-free translation of compound VII-2 (50 nM) against RTA (50 nM).

FIG. 6 details the results for a luciferase activity assay using yeast cell-free translation of compound I-3 (50 nM) against RTA (50 nM).

FIG. 7 details the results for a luciferase activity assay using yeast cell-free translation of compound I-5 (50 nM) against RTA (50 nM).

FIG. 8 details the results for a luciferase activity assay using yeast cell-free translation of compound II-3 (50 nM) against RTA (50 nM).

FIG. 9 details the results for a luciferase activity assay using yeast cell-free translation of compound II-13 (50 nM) against RTA (50 nM).

FIG. 10 details the results for a luciferase activity assay using yeast cell-free translation of compound VII-13 (50 nM) against RTA (50 nM).

FIG. 11 details the results for a luciferase activity assay using yeast cell-free translation of compound IV-10 (50 nM) against RTA (50 nM).

FIG. 12 details the results for a luciferase activity assay using yeast cell-free translation of compound II-2 (20 nM) against RTA (20 nM).

FIG. 13 details the results for a luciferase activity assay using yeast cell-free translation of compound IV-3 (20 nM) against RTA (20 nM).

FIG. 14 details the results for a luciferase activity assay using yeast cell-free translation of compound II-12 (20 nM) against RTA (20 nM).

FIG. 15 details the results for a luciferase activity assay using yeast cell-free translation of compound VII-3 (20 nM) against RTA (20 nM).

FIG. 16 details the results for a luciferase activity assay using yeast cell-free translation of compound V-21 (20 nM) against RTA (20 nM).

FIG. 17 details the results for a luciferase activity assay using yeast cell-free translation of compound IV-9 (20 nM) against RTA (20 nM).

FIG. 18 details the results for a luciferase activity assay using yeast cell-free translation of compound VII-1 (20 nM) against RTA (20 nM).

FIG. 19 details the results for a luciferase activity assay using yeast cell-free translation of compound V-1 (20 nM) against RTA (20 nM).

FIG. 20 details the results for a luciferase activity assay using yeast cell-free translation of compound IV-19 (20 nM) against RTA (20 nM).

FIG. 21 details the results for a luciferase activity assay using yeast cell-free translation of compound I-4 (20 nM) against RTA (20 nM).

FIG. 22 details the results for a luciferase activity assay using yeast cell-free translation of compound IV-1 (20 nM) against RTA (20 nM).

FIG. 23 details the results for a luciferase activity assay using yeast cell-free translation of compounds IV-3, V-21, IV-9, and IV-8 (R16, R19, R20, and R22, respectively) (10 nM) against Stx2 (10 nM).

FIG. 24 details the results for a colorimetric-based mouse myeloma cell viability assay of compounds IV-3, V-21, IV-9, and IV-8 (R16, R19, R20, and R22, respectively).

FIG. 25 details the results for a colorimetric-based Vero cell viability assay of compounds IV-3, V-21, IV-9, and IV-8 (R16, R19, R20, and R22, respectively).

FIG. 26 shows the activity of various RTA inhibitors.

FIG. 27 details the results for a colorimetric-based Vero cell viability assay of compounds IV-59 and IV-61 (WS-58 and JGP-17, respectively).

FIG. 28 is a block diagram of a computing system that can be used in connection with the data models and computer-implemented methods described in this document.

### DETAILED DESCRIPTION

This disclosure provides materials and methods for inhibiting a protein synthesis inactivating toxin. For example, small-molecule inhibitors of ricin, abrin, Shiga toxins, and Shiga-like toxins are provided. Methods for using such small-molecule inhibitors to treat, prevent, or ameliorate one or more symptoms of protein synthesis inactivating toxin poisoning are also provided. Kits and articles of manufacture containing one or more small-molecule inhibitors and accessory items are also provided. Further provided is a method of identifying inhibitors of protein synthesis inactivating toxins.

### A. DEFINITIONS

As used herein, “protein synthesis inactivating toxin” includes toxins that are ribonucleases, N-glycosidases, or ADP-ribosyltransferases. N-glycosidases are exemplified by the single polypeptide of the plant type I ribosome inactivating proteins (RIP) (e.g., gelonin, momordin, and saporin), the “A” chain of the plant type II ribosome-inactivating proteins (e.g., ricin, abrin, and modeccin), and similar acting bacterial toxins (e.g., Shiga toxins). The term “protein synthesis inactivating toxin” as used herein also includes specific ribonucleases that digest a specific phosphodiester bond in the backbone of ribosomal RNA, thereby inactivating the ribosomes and inhibiting protein synthesis. Ribonucleases are exemplified by the fungal toxins alpha-sarcin, mitogillin, and restrictocin, and also include similar acting bacterial toxins. The term “protein synthesis inactivating toxin” also includes the ADP-ribosylating component of the ADP-ribosyltransferases, which are proteolytically activated bacterial toxins that ADP-ribosylate, and thus inactivate, components of the protein synthesis machinery (e.g., diphtheria toxin and Pseudomonas exotoxin A).

Plant ribosome-inactivating proteins (RIPs) are N-glycosidases that cleave (i.e. depurinate) the N-glycosidic bond of adenine in a specific ribosomal RNA sequence. Many RIPs are single-chain proteins (type I RIPs), but some (type II RIPs) possess a galactose-specific lectin domain that binds to cell surfaces. The type II RIPs are potent toxins, and include, for example, ricin.

As used herein, “type II ribosome-inactivating proteins” or “type II RIPs” means two-chain N-glycosidases that cleave the N-glycosidic bond of adenine in a specific ribosomal RNA sequence, wherein the two chains are an A chain, which possesses the N-glycosidase activity, and a B chain, which comprises a galactose-specific lectin domain that binds to cell surfaces. Ricin is one example of a prototypical type II ribosome-inactivating protein, but other such type II RIPs include abrin (from Abrus precatrius), modeccin (from Adenia digtata), viscumin (from Viscum album), Shiga toxin (from S. dysenteriae), and volkensin (from Adenia volkensii).

As used herein, “ricin A chain” of “RTA” means an N-glycosidase of about 32 KDa that digests and inactivates 26S and 28S ribosomal RNA by cleavage of a specific adenine residue located within a highly conserved region of the 26S and 28S ribosomal RNA.

As used herein, “ricin B chain” or “RTB” means a galactose/N-acetylgalactosamine-binding lectin of about 34 KDa.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, “treatment” means any manner in which one or more of the symptoms of a protein synthesis inactivating toxin poisoning, e.g., ricin, abrin, a Shiga toxin, or a Shiga-like (e.g., E. coli 0157:57) toxin poisoning, are ameliorated or otherwise beneficially altered. Treatment also encompasses any pharmaceutical use of the compositions herein, such as uses for treating diseases, disorders, or ailments in which a protein synthesis inactivating toxin is implicated.

As used herein, “amelioration” of the symptoms of a particular disorder by administration of a particular compound or pharmaceutical composition refers to any lessening, whether permanent or temporary, lasting or transient that can be attributed to or associated with administration of the composition.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl,” “alkenyl” and “alkynyl” refer to carbon chains that may be straight or branched. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propenyl).

As used herein, “cycloalkyl” refers to a saturated mono- or multi-cyclic carbon ring system. The ring systems of the cycloalkyl groups may be composed of one ring or two or more rings which may be joined together in a fused, bridged or spiro-connected fashion. Examples include, but are not limited to, cyclopropyl, cyclobutyl, cyclopentyl, and cyclohexyl.

As used herein, “aryl” refers to aromatic monocyclic or multicyclic carbon groups. Aryl groups include, but are not limited to groups such as unsubstituted or substituted fluorenyl, phenyl, and naphthyl.

As used herein, “heteroaryl” refers to a monocyclic or multicyclic aromatic ring system where one or more, in some embodiments, 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, “heterocycloalkyl” refers to a monocyclic or multicyclic non-aromatic ring system, where one or more, in certain embodiments, 1 to 3, of the atoms in the ring system is a heteroatom, that is, an element other than carbon, including but not limited to, nitrogen, oxygen or sulfur.

As used herein, “aralkyl” refers to an alkyl group, as discussed above, having an aryl substituent, as discussed above. Non-limiting examples of an aralkyl groups include benzyl, p-nitrobenzyl, phenylethyl, diphenylmethyl, and triphenylmethyl.

As used herein, “heteroaralkyl” refers to an alkyl group, as discussed above, having a heteroaryl substituent, as discussed above. Non-limiting examples of an heteroaralkyl groups include (2-furyl)methyl, (3-furyl)methyl, (2-thienyl)methyl, (3-thienyl)methyl, (2-pyridyl)methyl, 1-methyl-1-(2-pyrimidyl)ethyl and the like.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen.

As used herein, “carboxy” refers to a divalent radical, —C(O)O—.

As used herein, “aminocarbonyl” refers to —C(O)NH₂.

As used herein, “aminoalkyl” refers to —RNH₂, in which R is alkyl.

As used herein, “alkoxy” and “alkylthio” refer to RO— and RS—, in which R is alkyl.

As used herein, “aryloxy” and “arylthio” refer to RO— and RS—, in which R is aryl.

As used herein, “amido” refers to the divalent group —C(O)NH—.

As used herein, “hydrazide” refers to the divalent group —C(O)NHNH—.

Where the number of any given substituent is not specified (e.g., haloalkyl), there may be one or more substituents present. For example, “haloalkyl” may include one or more of the same or different halogens.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

### B. COMPOUNDS

The compounds provided herein exhibit in vitro, ex vivo and in vivo activity against a protein synthesis inactivating toxin. In some embodiments, the compounds provided herein can inhibit a protein synthesis inactivating toxin. In some embodiments, the compounds provided herein can inhibit an N-glycosidase. In some embodiments, the compounds provided herein can inhibit a type II ribosome inhibiting protein (type II RIP). In some embodiments, the compounds provided herein can inhibit ricin, abrin, a Shiga toxin, or a Shiga-like toxin. In some embodiments, the compounds treat, prevent, or ameliorate one or more symptoms associated with a protein synthesis inactivating toxin poisoning, including ricin, abrin, a Shiga toxin, or a Shiga-like toxin poisoning.

Use of any of the compounds provided herein, or their pharmaceutically acceptable salts forms or derivatives, in the treatment or amelioration of a protein synthesis inactivating toxin poisoning, or associated disorders, is also provided, as well as use of any of the compounds, or pharmaceutically acceptable salts forms or derivatives, in the preparation of a medicament for the treatment or amelioration of a protein synthesis inactivating toxin poisoning.

Compounds for use in the compositions and methods provided herein, or pharmaceutically acceptable salt forms or derivatives thereof, can have Formula (I-A):

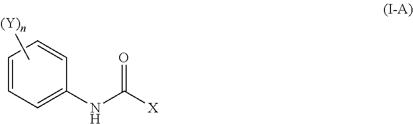

wherein:
X is selected from C₁₋₁₀ alkyl, C₅₋₁₂ cycloalkyl, C₅₋₁₂ aryl, or C₅₋₁₂ heteroaryl, wherein the alkyl, cycloalkyl, aryl, or heteroaryl may be substituted with one or more of C₁₋₁₀ alkyl, OR¹, NO₂, CONR¹R², COR¹, and halo;
each Y is independently H, C₁₋₁₀ alkyl, CO₂R¹, OR¹, or halo;
R¹ and R² are independently H, C₁₋₁₀ alkyl, and aryl; and
n is 1, 2, or 3.

In some embodiments, n is 2 wherein one Y is in the meta position on the ring and the other Y is in the para position on the ring. In some embodiments, Y in the meta position is a C₁₋₁₀ alkyl. In some embodiments, n is 3 wherein one Y is in the para position on the ring and the remaining two Y moieties are in the meta positions on the ring. In some embodiments, n is 1 wherein Y is in the para position. In some embodiments, Y is CO₂R¹. In some embodiments, Y is CO₂H.

In some embodiments, X is a C₁₋₁₀ alkyl. In some embodiments, X is a C₁₋₁₀ alkyl substituted with COR¹. In some embodiments, X is a C₅₋₁₂ heteroaryl. In some embodiments, X is quinolinyl. In some embodiments, X is a C₅₋₁₂ aryl.

In some embodiments, a compound according to Formula (I-A) can have Formula (I-B):

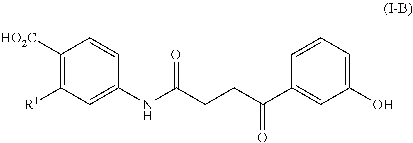

wherein:

R¹ is selected from H or C₁₋₁₀ alkyl.

In some embodiments, R¹ is selected from H, methyl, ethyl, or propyl.

In some embodiments, a compound according to Formula (I-A) can have Formula (I-C):

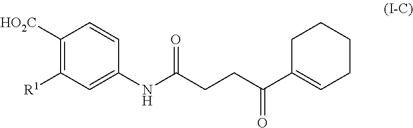

wherein:

R¹ is selected from H or C₁₋₁₀ alkyl.

In some embodiments, R¹ is selected from H, methyl, ethyl, or propyl.

In some embodiments, a compound according to Formula (I-A) can have Formula (I-D):

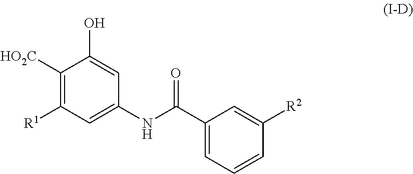

wherein:

R¹ is independently selected from H or C₁₋₁₀ alkyl; and

R² is selected from H, halo, or OR¹.

In some embodiments, R¹ is selected from H, methyl, ethyl, or propyl. In some embodiments, R² is selected from F and OH.

In some embodiments, a compound according for Formula (I-A) can be a compound according to Formula (I-E):

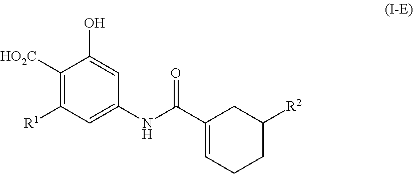

wherein:

R¹ is independently selected from H or C₁₋₁₀ alkyl; and

R² is selected from H, halo, or OR¹.

In some embodiments, R¹ is selected from H, methyl, ethyl, or propyl. In some embodiments, R² is selected from F and OH.

Exemplary compounds according to one or more of Formulas (I-A), (I-B), (I-C), (I-D), and (I-E) include:

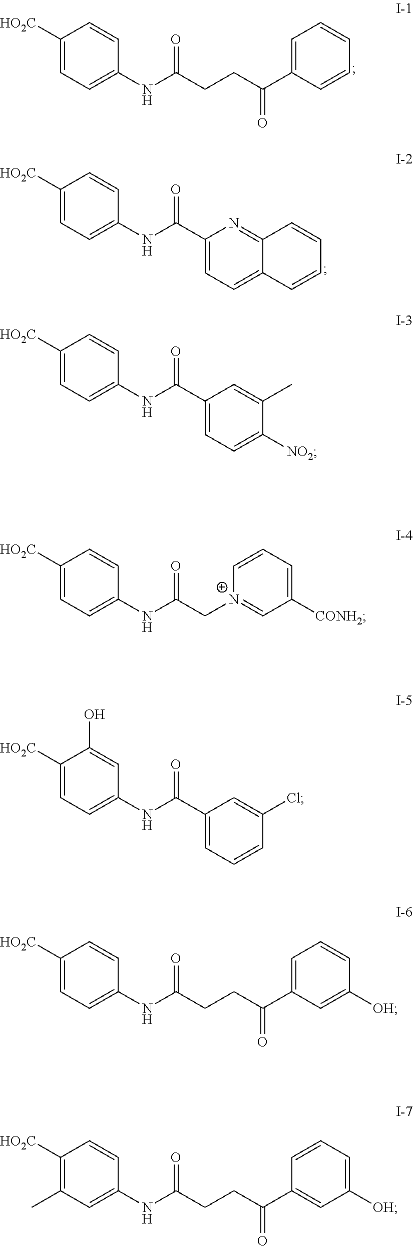

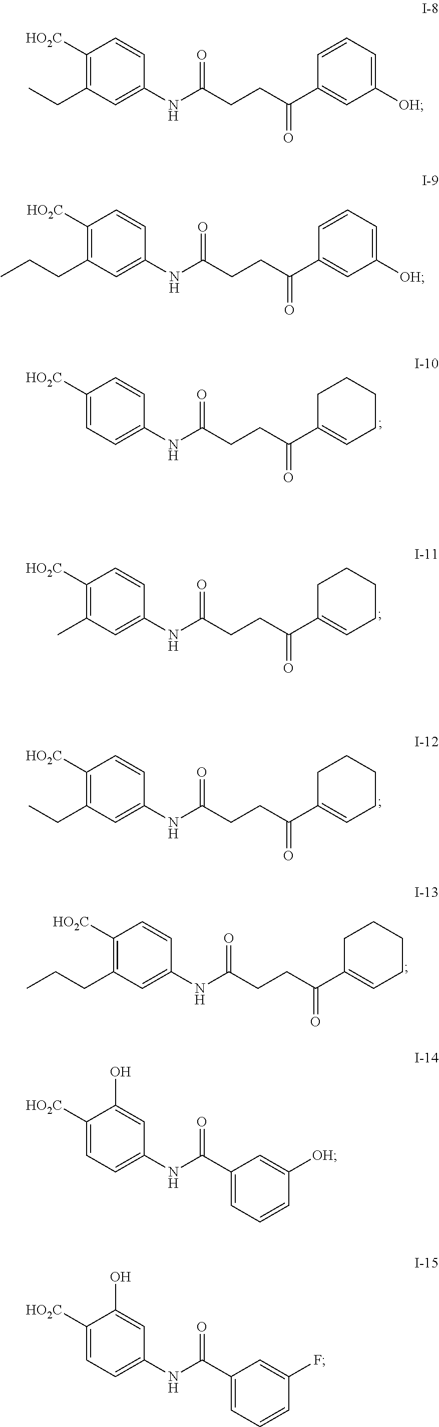

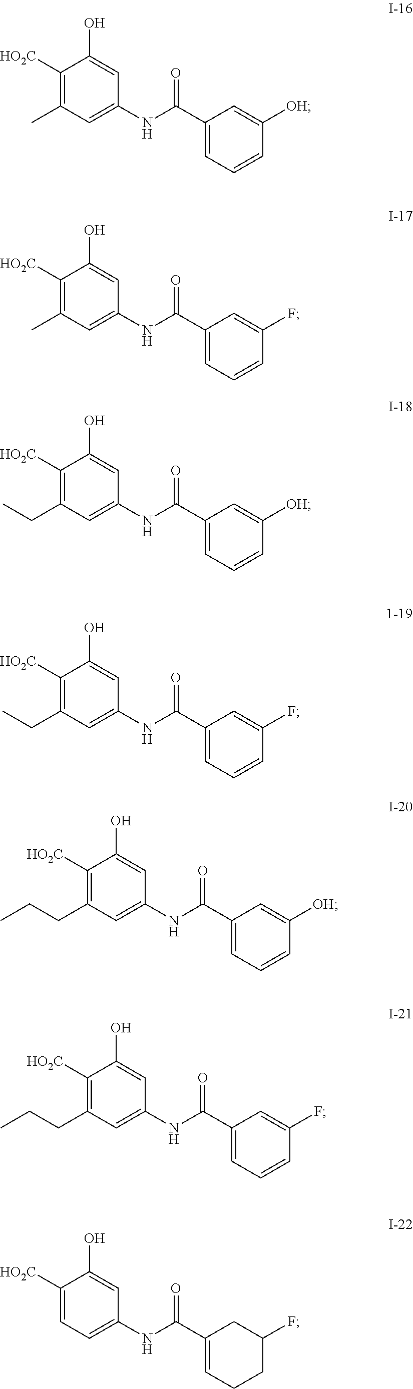

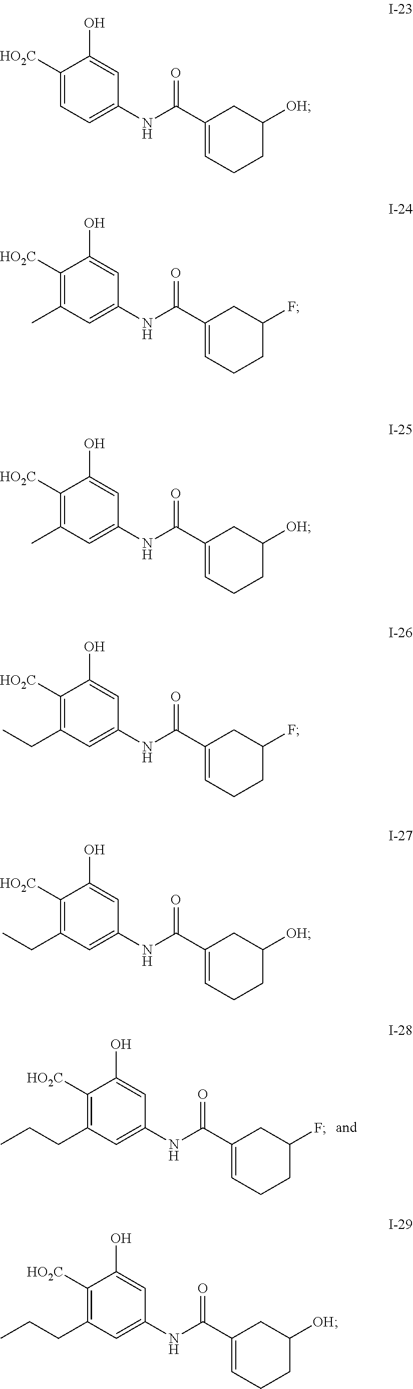

or a pharmaceutically acceptable salt form thereof.

In some embodiments, compounds for use in the compositions and methods provided herein, or pharmaceutically acceptable salt forms or derivatives thereof, can have Formula (II-A):

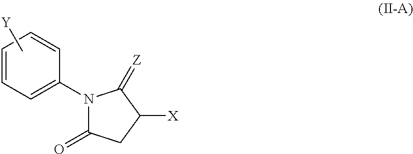

wherein:
X is selected from CO₂R¹, NR¹R², or C₅₋₁₂ heterocycloalkyl;
Y is selected from H, C₁₋₁₀ alkyl, OR¹, or halo;
Z is absent or O;
R¹ is H or C₁₋₁₀ alkyl; and
R² is selected from H, C₁₋₁₀ alkyl; and C₅₋₁₂ cycloalkyl, wherein the alkyl and cycloalkyl may be substituted with C₁₋₁₀ alkyl or C₅₋₁₂ heterocycloalkyl, wherein the heterocycloalkyl may be substituted with a C₁₋₁₀ alkyl.

In some embodiments, Y is H. In some embodiments, Y is F. In some embodiments, Y is Br. In some embodiments, Y is methyl. In some embodiments, Y is OR¹. In some embodiments, X is NR¹R². In some embodiments, X is C₅₋₁₂ heterocycloalkyl. In some embodiments, X is CO₂R¹.

In some embodiments, a compound according to Formula (II-A) can be a compound of Formula (II-B):

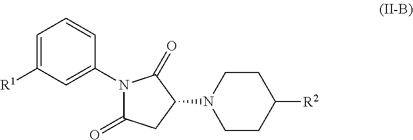

wherein:

R¹ is selected from H and OH; and

R² is selected from H or C₁₋₁₀ alkyl.

In some embodiments, R² is methyl, ethyl, or propyl.

Exemplary compounds according to one or more of Formulas (II-A) and (II-B) include:

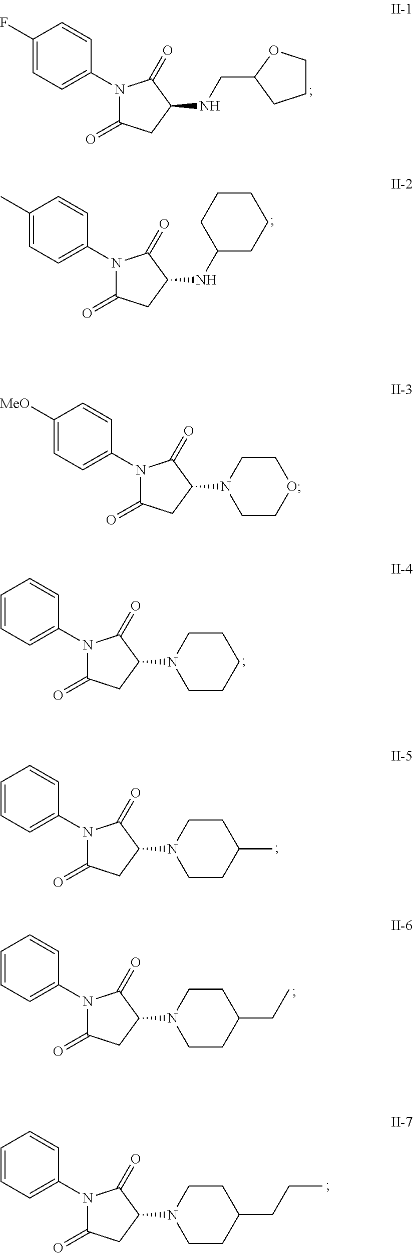

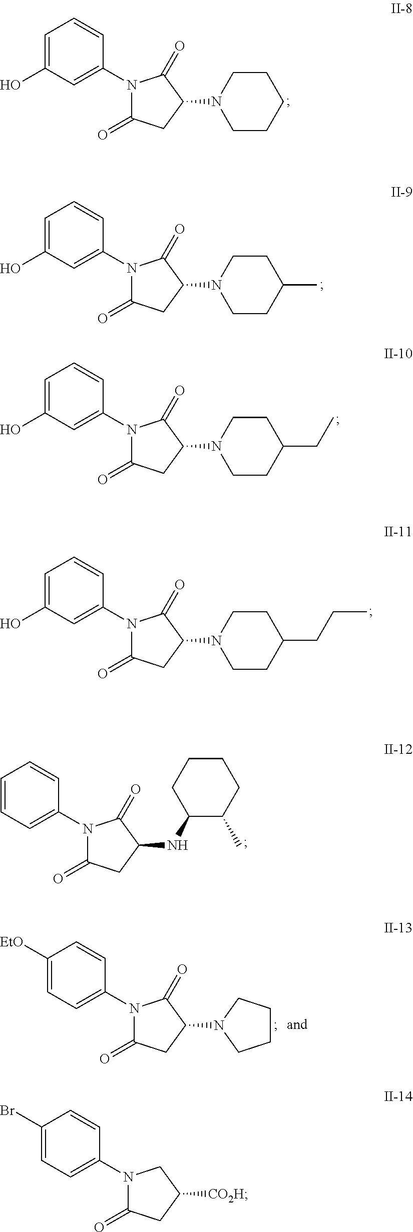

or a pharmaceutically acceptable salt form thereof.

Also provided herein are compounds for use in the compositions and methods provided herein, or pharmaceutically acceptable salt forms or derivatives thereof, having a composition according to Formula (III-A):

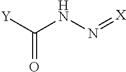

wherein:
X is C₁₋₁₀ alkyl, C₅₋₁₂ cycloalkyl, or C₅₋₁₂ heteroalkyl, wherein the alkyl and heteroaryl can be substituted with one or more of CO₂R¹, OR¹, and halo;
Y is selected from C₅₋₁₂ aryl, C₅₋₁₂ cycloalkyl, and C₅₋₁₂ heterocycle, wherein the heterocycle can be substituted with one or more of OR¹ and NR¹R²; and
R¹ and R² are independently selected from H and C₁₋₁₀ alkyl.

In some embodiments, Y is C₅₋₁₂ aryl. In some embodiments, Y is phenyl. In some embodiments, Y is C₅₋₁₂ heteroalkyl. In some embodiments, Y is C₅₋₁₂ cycloalkyl. In some embodiments, X is a C₅₋₁₂ heterocycloalkyl. In some embodiments, X is a C₁₋₁₀ alkyl.

In some embodiments, a compound according to Formula (III-A) can have Formula (III-B):

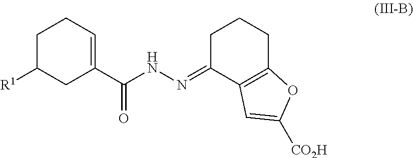

wherein:

R¹ is selected from H and OR²; and

R² is selected from H and C₁₋₁₀ alkyl.

In some embodiments, R¹ is H or OH.

Exemplary compounds according to one or more of Formulas (III-A) and (III-B) include:

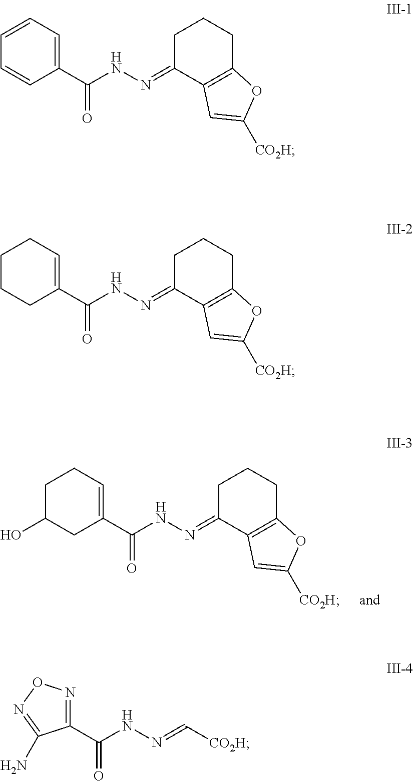

or a pharmaceutically acceptable salt form thereof.

In some embodiments, compounds for use in the compositions and methods provided herein, or pharmaceutically acceptable salt forms or derivatives thereof, can have Formula IV-A:

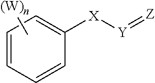

each W is independently C₁₋₁₀ alkyl, CO₂R¹, OR¹, or halo;
X is absent or NH;
Y is N or CH;
Z is selected from C₁₋₁₀ alkyl, C₁₋₁₀ alkenyl, C₁₋₁₀ aralkyl, C₁₋₁₀ heteroaralkyl, C₅₋₁₂ cycloalkyl, and C₅₋₁₂ heterocycle, wherein the alkyl, aralkyl, heteroaralkyl, and heterocycle can be substituted with one or more of C₁₋₁₀ alkyl, C(NH)NH₂, NR¹R², (CH₂)ₘNR¹R², OR¹, (CH₂)ₘOR¹, CN, NO₂, COR¹, CO₂R¹, CF₃, OCF₃, SO₃H, halo, and ═O;
R¹ and R² are independently selected from H, COCH₃, C₁₋₁₀ alkyl, (CH₂)ₘOH, and C₁₋₁₀ aryl;
m is an integer from one to three; and
n is an integer from one to three.

In some embodiments, W is C₁₋₁₀ alkyl. In some embodiments, W is CO₂R¹. In some embodiments, n is 1 and W is in the para position. In some embodiments, n is 1 and W is in the meta position. In some embodiments, n is 2 and the Ws are in ortho and meta positions. In some embodiments, X is NH. In some embodiments, Y is N. In some embodiments, Z is C₁₋₁₀ substituted or unsubstituted aralkyl. In some embodiments, Z is a C₁₋₁₀ heteroaralkyl. In some embodiments, Z is a C₅₋₁₂ cycloalkyl.

In some embodiments, a compound according to Formula (IV-A) is a compound of Formula (IV-B) or Formula (IV-C):

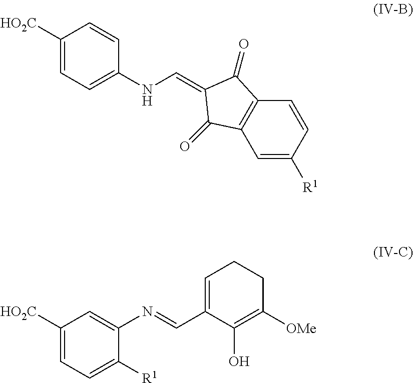

wherein:

R¹ is a C₁₋₁₀ alkyl.

In some embodiments, R¹ is selected from methyl, ethyl, and propyl.

In some embodiments, a compound according to Formula (IV-A) is a compound according to Formula (IV-D):

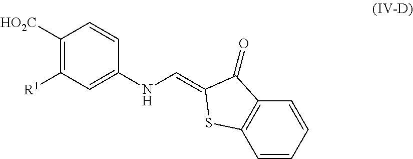

wherein:

R¹ is H or OR²; and

R² is H or C₁₋₁₀ alkyl.

In some embodiments, R¹ is H or OH.

Exemplary compounds according to one or more of Formulas (IV-A), (IV-B), (IV-C), and (IV-D) include:

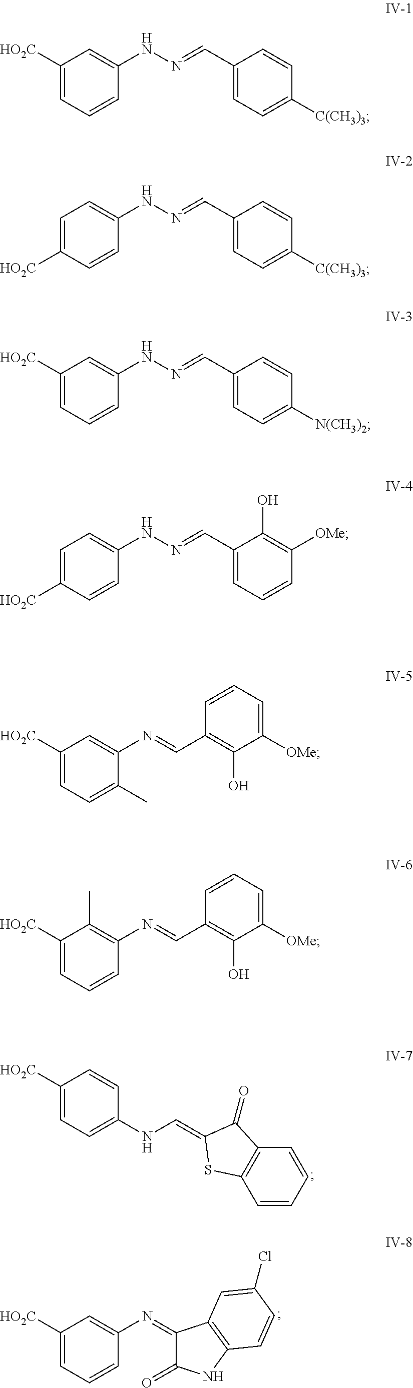

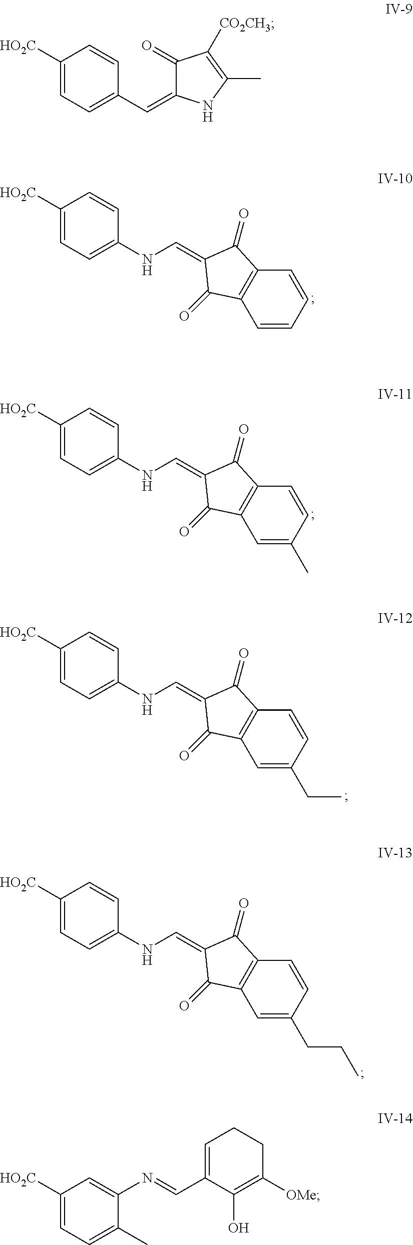

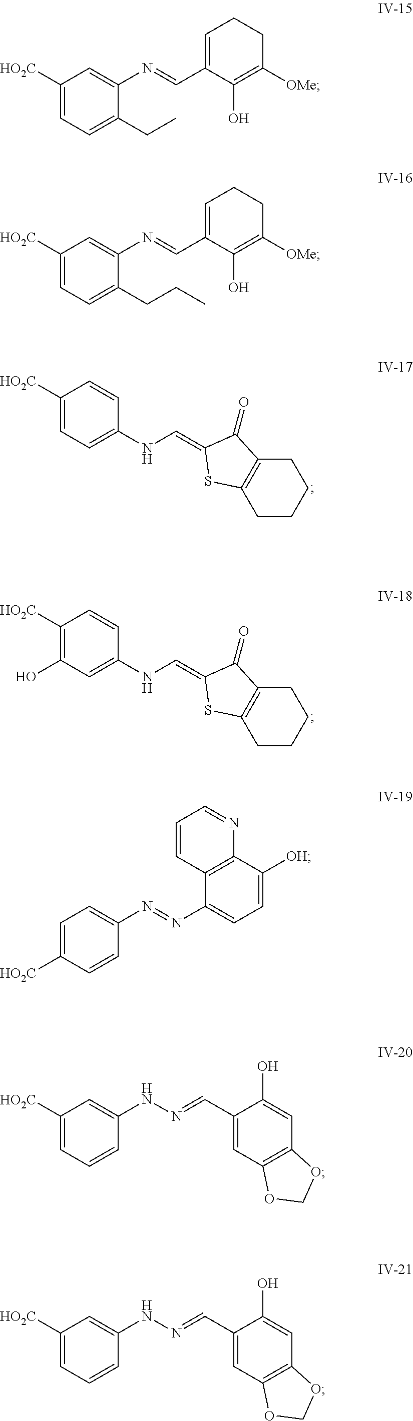

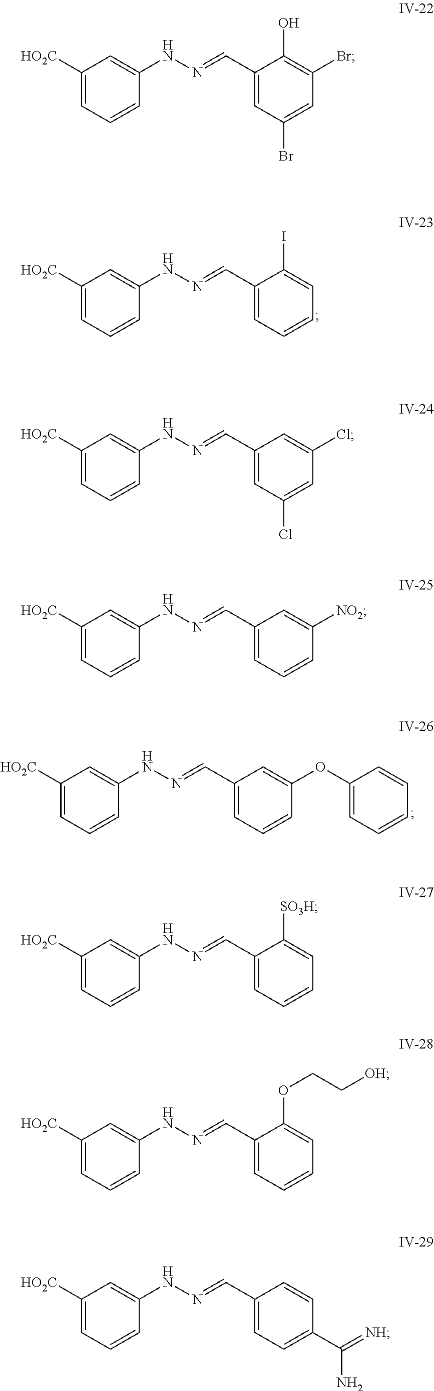

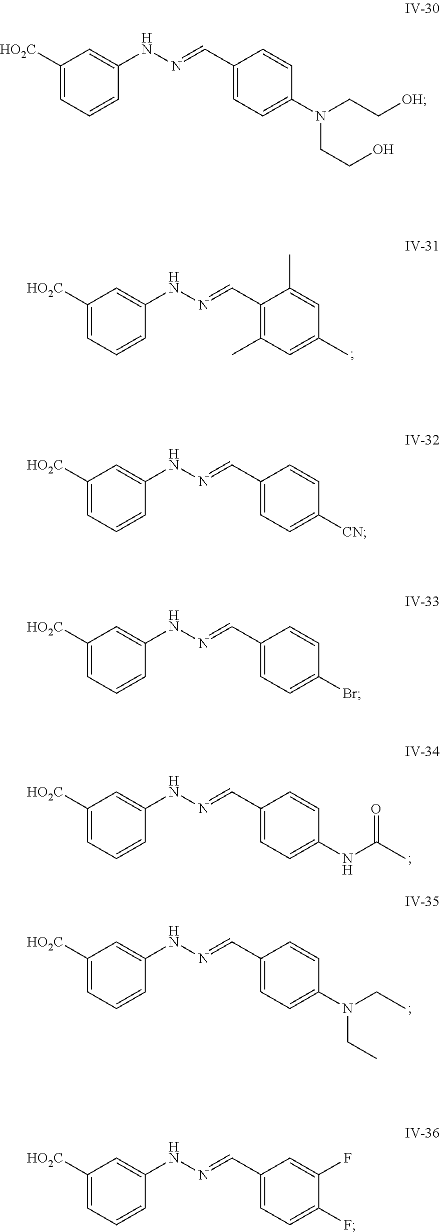

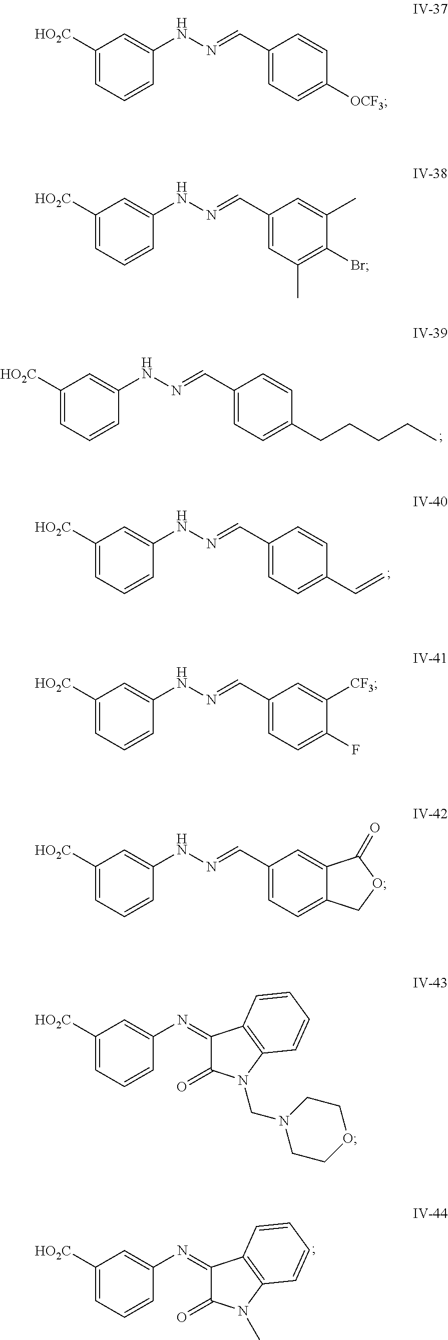

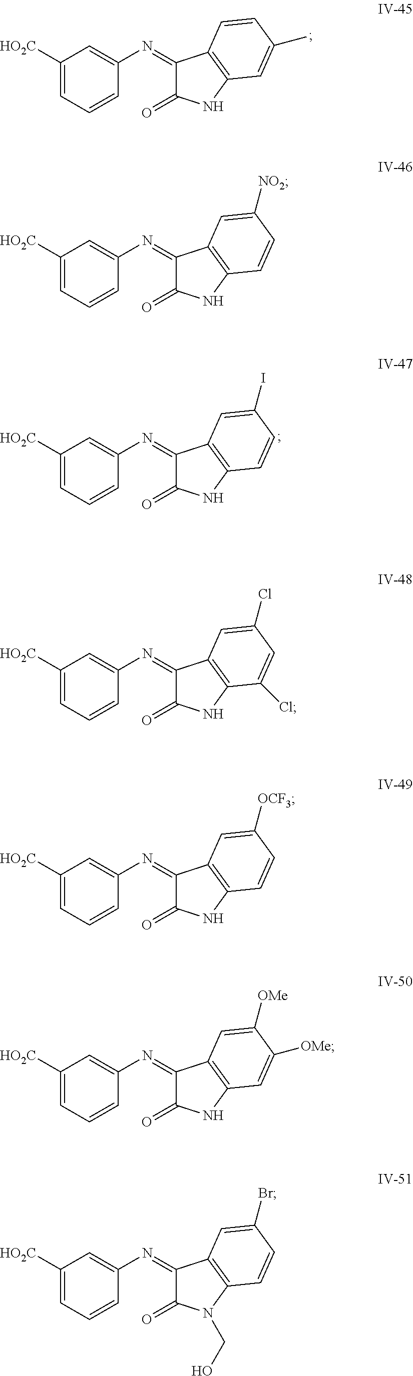

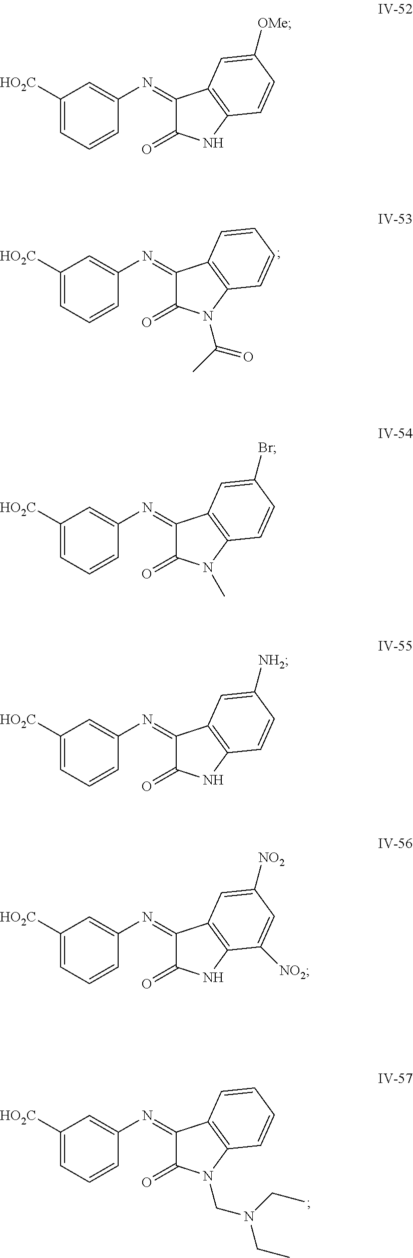

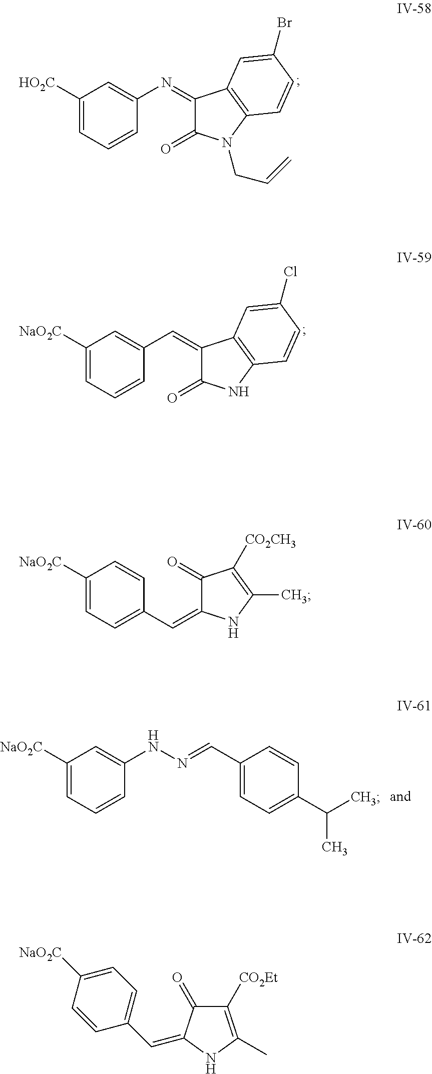

or a pharmaceutically acceptable salt form thereof.

In some embodiments, compounds for use in the compositions and methods provided herein, or pharmaceutically acceptable salt forms or derivatives thereof, can have Formula V-A:

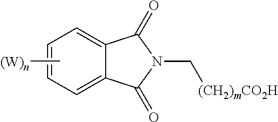

wherein
each W is independently C₁₋₁₀ alkyl, C₂₋₁₀ alkenyl, C₂₋₁₀ alkynyl, CO₂R¹, OR¹, halo, NO₂, NR¹R², or two W come together to form a fused aryl, heteroaryl, cycloalkyl, or heterocycloalkyl, wherein the alkyl, alkenyl or alkynyl can be unsubstituted or substituted with CO₂R¹, OR¹, or halo;
R¹ and R² are independently selected from H and C₁₋₁₀ alkyl;
m is an integer from zero to five; and
n is an integer from zero to three.

Exemplary compounds according to Formulas (V-A) include:

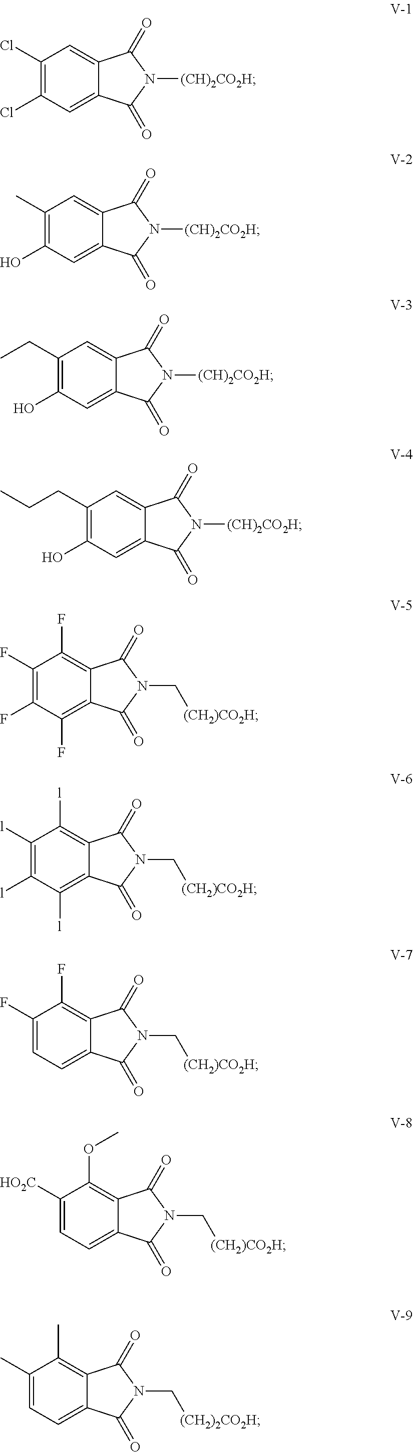

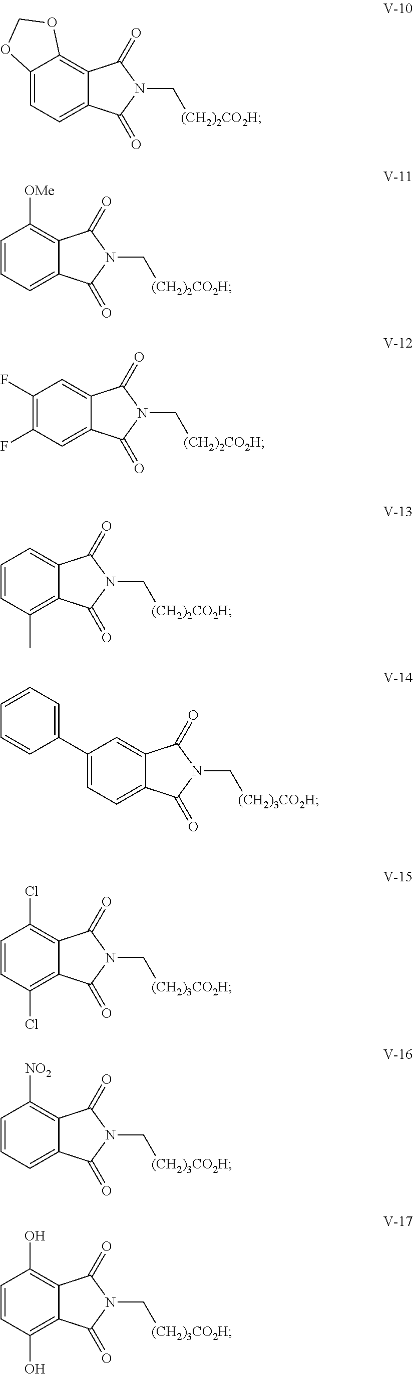

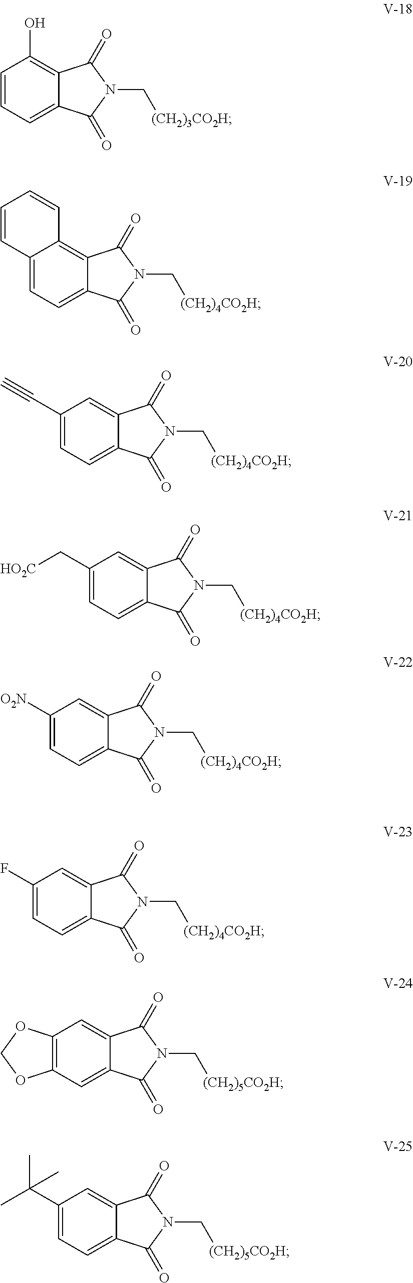

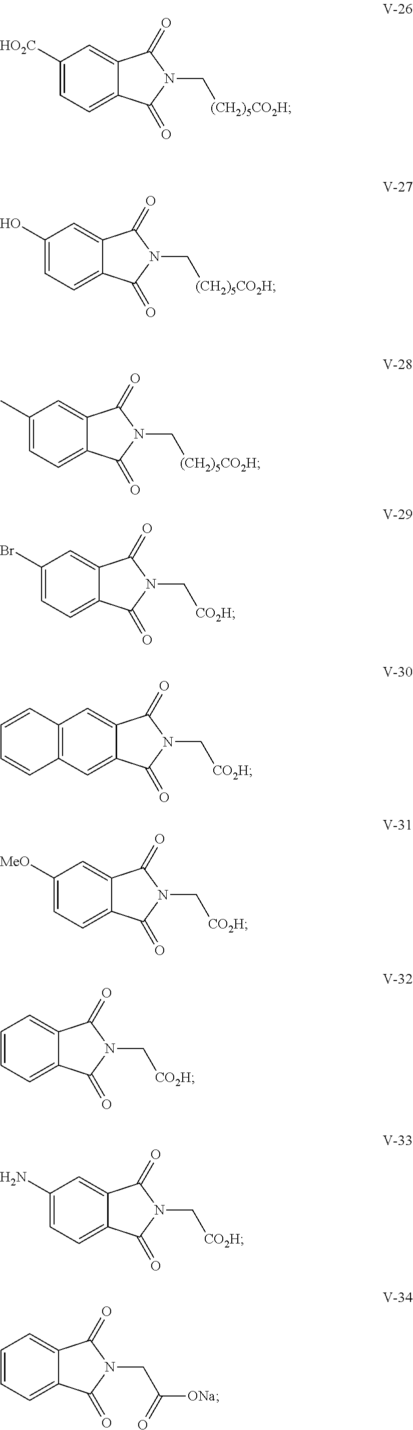

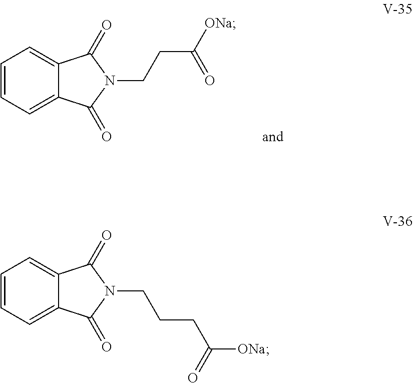

or a pharmaceutically acceptable salt form thereof.

In some embodiments, compounds for use in the compositions and methods provided herein, or pharmaceutically acceptable salt forms or derivatives thereof, can have Formula VI-A:

A-(CH₂)ₙ-B

wherein

A is selected from the group consisting of:

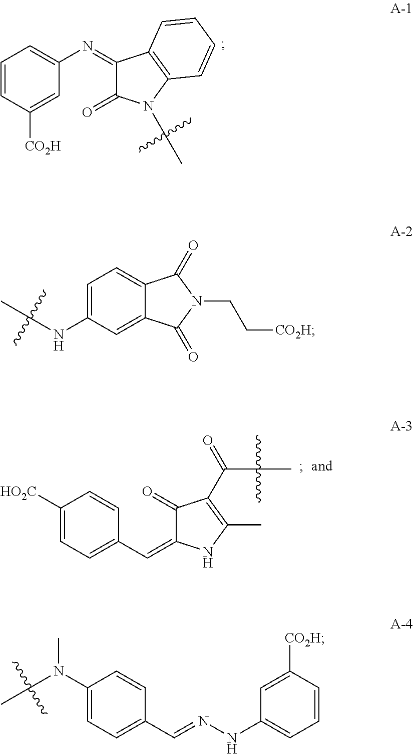

B is selected from the group consisting of:

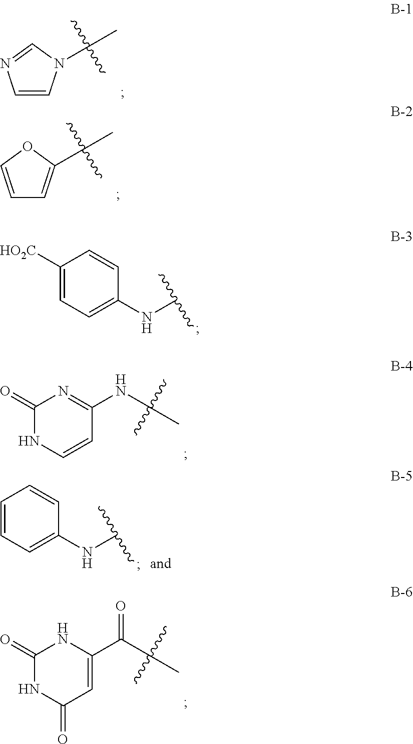

and

n is an integer from four to ten.

Exemplary compounds according to Formulas VI-A include:

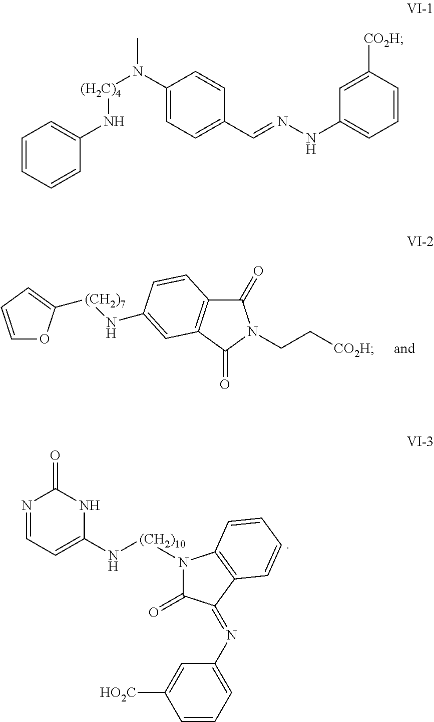

In some embodiments, compounds for use in the compositions and methods provided herein, or pharmaceutically acceptable salt forms or derivatives thereof, can be selected from:

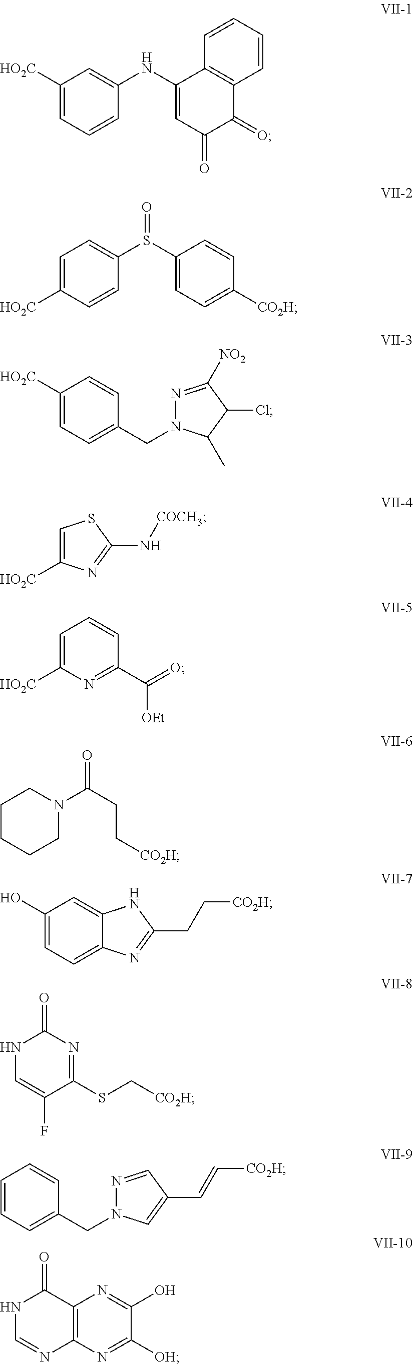

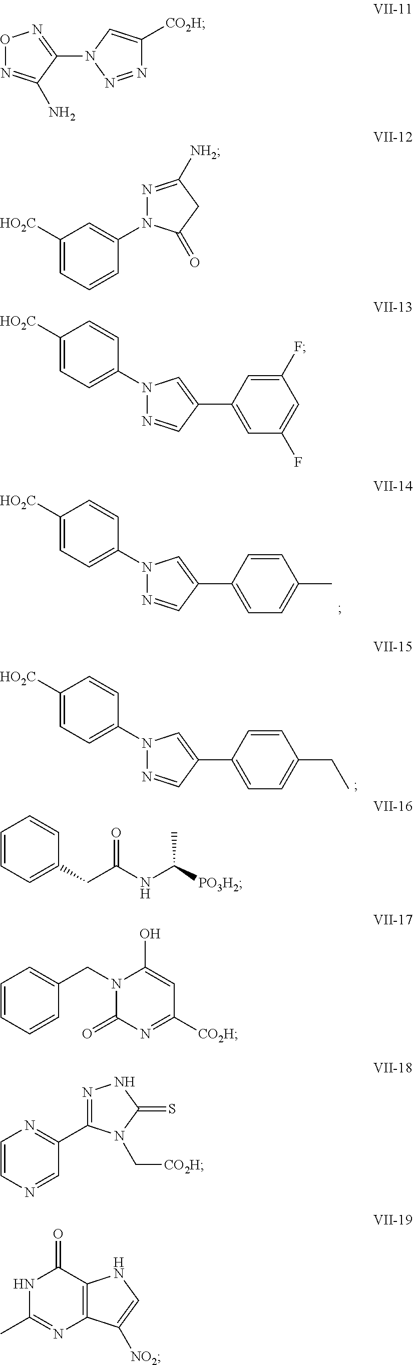

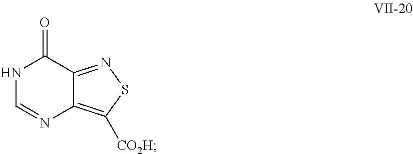

or a pharmaceutically acceptable salt form thereof.

### C. PREPARATION OF THE COMPOUNDS

The compounds for use in the compositions and methods provided herein may be obtained from commercial sources (e.g., Asinex or SpecsBiospecs), or may be prepared by the methods shown in the examples below and those known to persons of skill in the art.

### D. FORMULATION OF PHARMACEUTICAL COMPOSITIONS

The pharmaceutical compositions provided herein contain therapeutically effective amounts of one or more of the compounds provided herein that are useful in the treatment, prevention, or amelioration of one or more of the symptoms associated with a protein synthesis inactivating toxin poisoning, or a disorder or ailment in which a protein synthesis inactivating toxin poisoning is implicated, and a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

In some embodiments, the compositions contain one or more of the compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats or ameliorates one or more of the symptoms of N-glycosidase or type II ribosome inhibiting protein poisoning (e.g., ricin, abrin, a Shiga toxin, or a Shiga-like toxin poisoning).

In one embodiment, the compositions are formulated for single dosage administration. To formulate a composition, the weight fraction of compound is dissolved, suspended, dispersed or otherwise mixed in a selected carrier at an effective concentration such that the treated condition is relieved or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems, and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art.

Pharmaceutical dosage unit forms are prepared to provide from about 0.01 mg, 0.1 mg or 1 mg to about 200 mg, 1000 mg or 2000 mg, and in one embodiment from about 10 mg to about 200 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disorder being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, or in one embodiment 0.1-95%.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

a. Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action. The active ingredient is a compound or pharmaceutically acceptable derivative thereof as described herein. Higher concentrations, up to about 98% by weight of the active ingredient, may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-320-dimethyl ether, polyethylene glycol-520-dimethyl ether, polyethylene glycol-720-dimethyl ether wherein 320, 520 and 720 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

2. Injectables, Solutions, and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration should be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 20 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

6. Articles of Manufacture

The compounds or pharmaceutically acceptable derivatives may be packaged as articles of manufacture (e.g., kits) containing packaging material, a compound or pharmaceutically acceptable salt or derivative thereof provided herein within the packaging material, and a label that indicates that the compound or composition, or pharmaceutically acceptable derivative thereof, is useful for treatment, prevention, or amelioration of one or more symptoms or disorders in which a protein synthesis inactivating toxin poisoning, including ricin, abrin, a Shiga toxin, or a Shiga-like toxin poisoning, is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

7. Sustained Release Formulations

Also provided are sustained release formulations to deliver the compounds to the desired target at high circulating levels (between 10⁻⁹ and 10⁻⁴ M). The levels are either circulating in the patient systemically, or in one embodiment, localized to a site of, e.g., paralysis.

It is understood that the compound levels are maintained over a certain period of time as is desired and can be easily determined by one skilled in the art. Such sustained and/or timed release formulations may be made by sustained release means of delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556 and 5,733,566, the disclosures of which are each incorporated herein by reference. These pharmaceutical compositions can be used to provide slow or sustained release of one or more of the active compounds using, for example, hydroxypropylmethyl cellulose, other polymer matrices, gels, permeable membranes, osmotic systems, multilayer coatings, microparticles, liposomes, microspheres, or the like. Suitable sustained release formulations known to those skilled in the art, including those described herein, may be readily selected for use with the pharmaceutical compositions provided herein. Thus, single unit dosage forms suitable for oral administration, such as, but not limited to, tablets, capsules, gelcaps, caplets, powders and the like, that are adapted for sustained release are contemplated herein.

In one embodiment, the sustained release formulation contains active compound such as, but not limited to, microcrystalline cellulose, maltodextrin, ethylcellulose, and magnesium stearate. As described above, all known methods for encapsulation which are compatible with properties of the disclosed compounds are contemplated herein. The sustained release formulation is encapsulated by coating particles or granules of the pharmaceutical compositions provided herein with varying thickness of slowly soluble polymers or by microencapsulation. In one embodiment, the sustained release formulation is encapsulated with a coating material of varying thickness (e.g. about 1 micron to 200 microns) that allow the dissolution of the pharmaceutical composition about 48 hours to about 72 hours after administration to a mammal. In another embodiment, the coating material is a food-approved additive.

In another embodiment, the sustained release formulation is a matrix dissolution device that is prepared by compressing the drug with a slowly soluble polymer carrier into a tablet. In one embodiment, the coated particles have a size range between about 0.1 to about 300 microns, as disclosed in U.S. Pat. Nos. 4,710,384 and 5,354,556, which are incorporated herein by reference in their entireties. Each of the particles is in the form of a micromatrix, with the active ingredient uniformly distributed throughout the polymer.

Sustained release formulations such as those described in U.S. Pat. No. 4,710,384, which is incorporated herein by reference in its entirety, having a relatively high percentage of plasticizer in the coating in order to permit sufficient flexibility to prevent substantial breakage during compression are disclosed. The specific amount of plasticizer varies depending on the nature of the coating and the particular plasticizer used. The amount may be readily determined empirically by testing the release characteristics of the tablets formed. If the medicament is released too quickly, then more plasticizer is used. Release characteristics are also a function of the thickness of the coating. When substantial amounts of plasticizer are used, the sustained release capacity of the coating diminishes. Thus, the thickness of the coating may be increased slightly to make up for an increase in the amount of plasticizer. Generally, the plasticizer in such an embodiment will be present in an amount of about 15 to 30% of the sustained release material in the coating, in one embodiment 20 to 25%, and the amount of coating will be from 10 to 25% of the weight of the active material, and in another embodiment, 15 to 20% of the weight of active material. Any conventional pharmaceutically acceptable plasticizer may be incorporated into the coating.

The compounds provided herein can be formulated as a sustained and/or timed release formulation. All sustained release pharmaceutical products have a common goal of improving drug therapy over that achieved by their non-sustained counterparts. Ideally, the use of an optimally designed sustained release preparation in medical treatment is characterized by a minimum of drug substance being employed to cure or control the condition. Advantages of sustained release formulations may include: 1) extended activity of the composition, 2) reduced dosage frequency, and 3) increased patient compliance. In addition, sustained release formulations can be used to affect the time of onset of action or other characteristics, such as blood levels of the composition, and thus can affect the occurrence of side effects.

The sustained release formulations provided herein are designed to initially release an amount of the therapeutic composition that promptly produces the desired therapeutic effect, and gradually and continually release of other amounts of compositions to maintain this level of therapeutic effect over an extended period of time. In order to maintain this constant level in the body, the therapeutic composition must be released from the dosage form at a rate that will replace the composition being metabolized and excreted from the body.

The sustained release of an active ingredient may be stimulated by various inducers, for example pH, temperature, enzymes, water, or other physiological conditions or compounds.

Preparations for oral administration may be suitably formulated to give controlled release of the active compound. In one embodiment, the compounds are formulated as controlled release powders of discrete microparticles that can be readily formulated in liquid form. The sustained release powder comprises particles containing an active ingredient and optionally, an excipient with at least one non-toxic polymer.

The powder can be dispersed or suspended in a liquid vehicle and will maintain its sustained release characteristics for a useful period of time. These dispersions or suspensions have both chemical stability and stability in terms of dissolution rate. The powder may contain an excipient comprising a polymer, which may be soluble, insoluble, permeable, impermeable, or biodegradable. The polymers may be polymers or copolymers. The polymer may be a natural or synthetic polymer. Natural polymers include polypeptides (e.g., zein), polysaccharides (e.g., cellulose), and alginic acid. Representative synthetic polymers include those described, but not limited to, those described in column 3, lines 33-45 of U.S. Pat. No. 5,354,556, which is incorporated by reference in its entirety. Particularly suitable polymers include those described, but not limited to those described in column 3, line 46-column 4, line 8 of U.S. Pat. No. 5,354,556 which is incorporated by reference in its entirety.

The sustained release compositions provided herein may be formulated for parenteral administration, e.g., by intramuscular injections or implants for subcutaneous tissues and various body cavities and transdermal devices. In one embodiment, intramuscular injections are formulated as aqueous or oil suspensions. In an aqueous suspension, the sustained release effect is due to, in part, a reduction in solubility of the active compound upon complexation or a decrease in dissolution rate. A similar approach is taken with oil suspensions and solutions, wherein the release rate of an active compound is determined by partitioning of the active compound out of the oil into the surrounding aqueous medium. Only active compounds which are oil soluble and have the desired partition characteristics are suitable. Oils that may be used for intramuscular injection include, but are not limited to, sesame, olive, arachis, maize, almond, soybean, cottonseed and castor oil.

A highly developed form of drug delivery that imparts sustained release over periods of time ranging from days to years is to implant a drug-bearing polymeric device subcutaneously or in various body cavities. The polymer material used in an implant, which must be biocompatible and nontoxic, include but are not limited to hydrogels, silicones, polyethylenes, ethylene-vinyl acetate copolymers, or biodegradable polymers.

### E. EVALUATION OF THE ACTIVITY OF THE COMPOUNDS

The activity of the compounds provided herein as inhibitors of a protein synthesis inactivating toxin (e.g., ricin, abrin, a Shiga toxin, or a Shiga-like toxin) may be measured in a luminometer assay, e.g., those described in Iizuka, N. and Sarnow, P., Methods: A Companion to Methods in Enzymology 11(4):353-360 (1997). In one example, a test compound can be incubated with ricin A chain (RTA) and the mixture added to an assay mixture containing a yeast cell-free translation competent extract and capped lucierase RNA. The assay can be stopped by the addition of 100 μL TBS buffer. The amount of active luciferase protein (indicating translation efficiency of the in vitro reaction) can be measured using a luminometer following addition of a luciferase assay reagent. As a negative control, cycloheximide can be used as a translation inhibitor in the in vitro assay instead of ricin, to identify small molecules that may affect other steps in translation (e.g., other than ribosome depurination).

The activity of the compounds provided herein as inhibitors of a protein synthesis inactivating toxin (e.g., ricin, abrin, a Shiga toxin, or a Shiga-like toxin) may also be measured using a rabbit reticulocyte in vitro translation assay. In some embodiments, an RRL assay can be performed using 2:1 RRL obtained from Green Hectares (Oregon, Wis.). The RRL can be supplemented with the same ATP regeneration system used for the yeast in vitro translation assays discussed above, and the assay performed identically except the reaction is incubated at 30° C. for 1 hour.

Inhibitors of protein synthesis inactivating toxins can be evaluated using three different types of assays: (a) a neutralization assay (cells+inhibitor(s)+toxin mixed together at the same time); (b) a pre-treat assay (cells+inhibitor(s) preincubated before toxin challenge); and (c) a rescue assay (cells+toxin preincubated for some time before adding inhibitor(s)). In some embodiments, the antagonist(s) can be incubated with 1e4 Sp2/0-Ag14 (Sp2) mouse myeloma cells in hybridoma serum-free medium for 3 hrs at 37° C. in 96-well microplates. The toxin can be added to the cells to yield 40 pg/mL final concentration and the mixtures further incubated overnight. Metabolic activity of the cells can be determined using a CellTiter 96 Aqueous Cell Proliferation Assay (Promega). In some embodiments, the results are expressed in percent of the metabolic activity of Sp2 or Vera cells incubated under the same conditions in the absence of toxin and antagonist(s).

### F. METHODS OF USE OF THE COMPOUNDS AND COMPOSITIONS

Provided herein are methods to treat, prevent, or ameliorate symptoms or disorders associated with a protein synthesis inactivating toxin poisoning, including ricin, abrin, a Shiga toxin, or a Shiga-like toxin poisoning. The methods include administering one or more of the compounds described herein, or a pharmaceutically acceptable salt form or derivative thereof, to a mammal, e.g., a human, cat, dog, horse, pig, cow, sheep, mouse, rat, or monkey.

In certain embodiments, the symptoms or disorders associated with a protein synthesis inactivating toxin may depend on the route of exposure. In some embodiments, exposure to a protein synthesis inactivating toxin may occur via inhalation, ingestion, or injection. In some embodiments, the symptoms or disorders associated with a protein synthesis inactivating toxin poisoning may depend on the dose received. In some embodiments, the symptoms or disorders associated with a protein synthesis inactivating toxin poisoning include one or more of respiratory distress (difficulty breathing), fever, cough, nausea, tightness in the chest, heavy sweating, fluid build-up in the lungs (pulmonary edema), low blood pressure, respiratory failure, vomiting, diarrhea, severe dehydration, hallucinations, seizures, blood in the urine, liver failure, spleen failure, and kidney failure.

In practicing the methods, effective amounts of the compounds or composition provided herein are administered. Such amounts are sufficient to achieve a therapeutically effective concentration of the compound or active component of the composition in vivo.

The compounds described herein can also be used in combination with one or more one or more type II ribosome inactivating protein vaccines. In some embodiments, such combinations can be used to inhibit type II ribosome inactivating protein poisoning; reduce incapacitating local tissue damage at a portal of a type II ribosome inactivating protein entry; and/or reduce incapacitating lung damage in a subject.

### G. METHODS OF DESIGNING INHIBITORS TARGETING THE RICIN ACTIVE SITE

Provided herein are methods, including computer-based methods, for designing compounds that bind to and/or inhibit an active site of ricin as set forth in the crystal structure having PDB code 1IFS. The active site of ricin includes, but is not limited to, the residues of region I and/or II in the active site of the crystal structure 1IFS. Region I includes residues Asp100, Ile-104, Asp75, Asn78, Tyr80, Val82, Phe93, Gly120, Gly121, Asn122, His94, Pro95, and Asp96 having the conformations as set forth in the 1IFS crystal structure. Region II includes residues Tyr80, Val81, Phe93, Gly121, Asn122, Tyr123, Ile172, Arg180, Ala79, Ser176, Glu177, and Leu126 having the conformations as set forth in the 1IFS crystal structure. Inhibitors bound in Region I (see Region-I inhibitors of Example 8) can be tethered to inhibitors anchored in Region II (see Region-II inhibitors of Example 8) to form more potent and selective heterodimeric inhibitors (see Example 8)

The inventors have determined that the conformations of residues in region I and/or region II, as found in crystal structure 1IFS, are useful for determining inhibitors with high affinity for the active site. Thus, given the three-dimensional model described herein as well as the identification of region I and II in the proper configuration as useful residues to target, one having ordinary skill in the art would know how to use standard molecular modeling or other techniques to identify peptides, peptidomimetics, and small-molecules that would bind to or interact with one or more of the residues in region I and/or II. In addition, one having ordinary skill in the art would be able to combine targeting such residues with the targeting of other amino acids (e.g., Arg213) that are located at the rim of the ricin active site.

By “molecular modeling” is meant quantitative and/or qualitative analysis of the structure and function of physical interactions based on three-dimensional structural information and interaction models. This includes conventional numeric-based molecular dynamic and energy minimization models, interactive computer graphic models, modified molecular mechanics models, distance geometry and other structure-based constraint models. Molecular modeling typically is performed using a computer and may be further optimized using known methods. See Example 1 below.

Methods of designing compounds that bind specifically (e.g., with high affinity) to one or more of the residues described previously typically are also computer-based, and involve the use of a computer having a program capable of generating an atomic model. Computer programs that use X-ray crystallography data or molecular model coordinate data, such as the data that are available from the PDB, are particularly useful for designing such compounds. Programs such as RasMol, for example, can be used to generate a three dimensional model. Computer programs such as INSIGHT (Accelrys, Burlington, Mass.), Auto-Dock (Accelrys), and Discovery Studio 1.5 (Accelrys) allow for further manipulation and the ability to introduce new structures.

Compounds can be designed using, for example, computer hardware or software, or a combination of both. However, designing is preferably implemented in one or more computer programs executing on one or more programmable computers, each containing a processor and at least one input device. The computer(s) preferably also contain(s) a data storage system (including volatile and non-volatile memory and/or storage elements) and at least one output device. Program code is applied to input data to perform the functions described above and generate output information. The output information is applied to one or more output devices in a known fashion. The computer can be, for example, a personal computer, microcomputer, or work station of conventional design.

Each program is preferably implemented in a high level procedural or object oriented programming language to communicate with a computer system. However, the programs can be implemented in assembly or machine language, if desired. In any case, the language can be a compiled or interpreted language.

Each computer program is preferably stored on a storage media or device (e.g., ROM or magnetic diskette) readable by a general or special purpose programmable computer. The computer program serves to configure and operate the computer to perform the procedures described herein when the program is read by the computer. The method of the invention can also be implemented by means of a computer-readable storage medium, configured with a computer program, where the storage medium so configured causes a computer to operate in a specific and predefined manner to perform the functions described herein.

For example, a computer-assisted method of generating a test inhibitor of the active site of ricin as set forth by the crystal structure 1IFS is provided. The method uses a programmed computer comprising a processor and an input device, and can include:

(a) inputting on the input device, e.g., through a keyboard, a diskette, or a tape, data (e.g. atomic coordinates) comprising a docking box surrounded by one or more one residues of the active site of ricin as defined by the 1IFS crystal structure;

(b) docking into the docking box a test inhibitor molecule using the processor; and

(c) determining, based on the docking, whether the test inhibitor molecule would be capable of interacting with the one or more residues of the active site.

In some embodiments, the method uses a programmed computer comprising a processor, and can include:

(a) receiving data (e.g. atomic coordinates) comprising a docking box surrounded by one or more one residues of the active site of ricin as defined by the 1IFS crystal structure at a computing device;

(b) docking into the docking box a test inhibitor molecule using the processor; and

(c) determining in the computing device, based on the docking, whether the test inhibitor molecule would be capable of interacting with the one or more residues of the active site.

In some embodiments, the method can further include storing in a computer memory storage location the results of docking a test inhibitor molecule into the docking box (e.g., interaction energy values and binding strengths).

In some embodiments, the docking box is surrounded by one or more of the residues Asp100, Ile-104, Asp75, Asn78, Tyr80, Val82, Phe93, Gly120, Gly121, Asn122, His94, Pro95, and Asp96 having conformations as set forth in the 1IFS crystal structure. In some embodiments, the docking box is surrounded by one or more of residues Tyr80, Val81, Phe93, Gly121, Asn122, Tyr123, Ile172, Arg180, Ala79, Ser176, Glu177, and Leu126 having confirmations as set forth in the 1IFS crystal structure. In some embodiments, the test inhibitor molecule is capable of interacting with one or more of residues Asp100, Ile-104, Asp75, Asn78, Tyr80, Val82, Phe93, Gly120, Gly121, Asn122, His94, Pro95, and Asp96 having conformations as set forth in the 1IFS crystal structure. In some embodiments, the test inhibitor molecule is capable of interacting with one or more of residues Tyr80, Val81, Phe93, Gly121, Asn122, Tyr123, Ile172, Arg180, Ala79, Ser176, Glu177, and Leu126 having confirmations as set forth in the 1IFS crystal structure.

By “capable of interacting” it is meant capable of forming a one or more hydrogen bonds, ionic bonds, covalent bonds, pi-pi interactions, cation-pi interactions, sulfur-aromatic interactions, or VdW interactions. In some embodiments, the test inhibitor molecule can interact with one or more residues (e.g., one or more residues of region I or II) of the active site of ricin with a minimum interaction energy of −5 to about −50 kcal/mol, e.g., −20 to −40 kcal/mol. In some embodiments, the test inhibitor would be capable of forming a hydrogen bond with one or more residues of the active site of ricin.

The inhibitory activity of the test inhibitor on ricin in vitro can be evaluated. In some embodiments, the inhibitory activity is evaluated using one or more of a luminometer assay; an RRL assay; a neutralization assay; a pre-treat assay; and a rescue assay (see Examples 2-4).

From the information obtained using these methods, one skilled in the art will be able to design and make inhibitory compounds (e.g., peptides, non-peptide small molecules, peptidomimetics, and aptamers (e.g., nucleic acid aptamers)) with the appropriate 3-D structure, e.g., at certain residues and that interact in certain manners (e.g., hydrogen-bonding, ion bonding, covalent bonding, pi-pi interactions, sulfur-aromatic interactions, steric interactions, and/or van der Waals interactions). For example, one of skill in the art could design inhibitory compounds that could interact with one or more of the residues corresponding to Asp100, Ile-104, Asp75, Asn78, Tyr80, Val82, Phe93, Gly120, Gly121, Asn122, His94, Pro95, and Asp96, or residues Tyr80, Val81, Phe93, Gly121, Asn122, Tyr123, Ile172, Arg180, Ala79, Ser176, Glu177, and Leu126 whose confirmations are as defined in the 1IFS crystal structure.

Moreover, if computer-usable 3-D data (e.g., x-ray crystallographic data) for a candidate compound are available, one or more of the following computer-based steps can be performed in conjunction with computer-based steps described above:

(d) inputting into an input device, e.g., through a keyboard, a diskette, or a tape, data (e.g. atomic coordinates) that define the three-dimensional (3-D) structure of a candidate compound;

(e) determining, using a processor, the 3-D structure (e.g., an atomic model) of the candidate compound;

(f) determining, using the processor, whether the candidate compound binds to or interacts with one or more of the residues of interest in the ricin active site;

(g) determining the interaction energy of the candidate compound;

(h) identifying the candidate compound as a compound that inhibits the site;

(j) receiving, through an input device, e.g., through a keyboard, a diskette, or a tape, data (e.g. atomic coordinates) that define the three-dimensional (3-D) structure of a candidate compound at a computing device;

(k) determining, using the computing device, the 3-D structure (e.g., an atomic model) of the candidate compound;

(l) determining, using the computing device, whether the candidate compound binds to or interacts with one or more of the residues of interest in the ricin active site;

(m) determining, using the computing device, the interaction energy of the candidate compound; and

(n) identifying, using the computing device, the candidate compound as a compound that inhibits the site.

The method can involve an additional step of outputting to an output device a model of the 3-D structure of the compound. The method can also involve an additional step of storing in a computer memory storage location the results of any step of the method. In addition, the 3-D data of candidate compounds can be compared to a computer database of, for example, 3-D structures stored in a data storage system. In some embodiments, the interaction energy of the candidate compound is less than −54 kcal/mol.

Candidate compounds identified as described above can then be tested in standard cellular inhibition assays familiar to those skilled in the art.

The 3-D structure of molecules can be determined from data obtained by a variety of methodologies. These methodologies include: (a) x-ray crystallography; (b) nuclear magnetic resonance (NMR) spectroscopy; (c) molecular modeling methods, e.g., homology modeling techniques, threading algorithms, and in particular the refined homology modeling methods described below in Example 1.

Any available method can be used to construct a 3-D model of the ricin active site from the x-ray crystallographic, molecular modeling, and/or NMR data using a computer as described above. Such a model can be constructed from analytical data points inputted into the computer by an input device and by means of a processor using known software packages, e.g., CATALYST (Accelrys), INSIGHT (Accelrys) and CeriusII, HKL, MOSFILM, XDS, CCP4, SHARP, PHASES, HEAVY, XPLOR, TNT, NMRCOMPASS, NMRPIPE, DIANA, NMRDRAW, FELIX, VNMR, MADIGRAS, QUANTA, BUSTER, SOLVE, O, FRODO, or CHAIN. The model constructed from these data can be visualized via an output device of a computer, using available systems, e.g., Silicon Graphics, Evans and Sutherland, SUN, Hewlett Packard, Apple Macintosh, DEC, IBM, or Compaq.

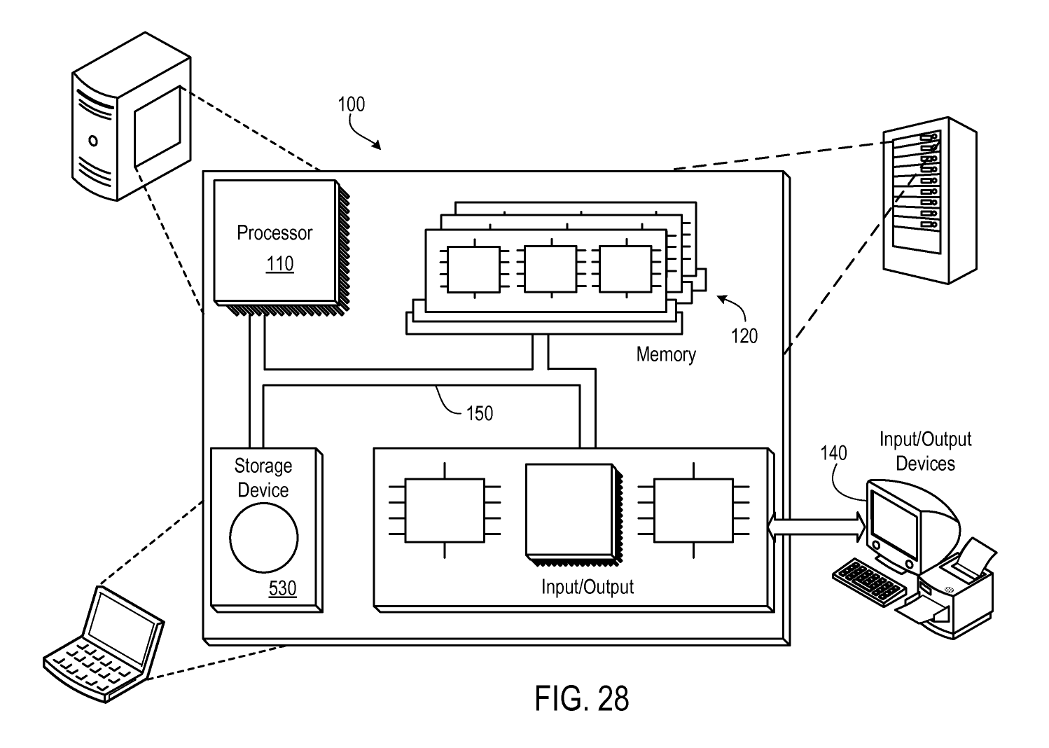

FIG. 28 is a schematic diagram of a computer system 100. The system 100 can be used for the operations described in association with any of the computer-implement methods described previously, according to one embodiment. The system 100 is intended to include various forms of digital computers, such as laptops, desktops, workstations, personal digital assistants, servers, blade servers, mainframes, and other appropriate computers. The system 100 can also include mobile devices, such as personal digital assistants, cellular telephones, smartphones, and other similar computing devices. Additionally the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The system 100 includes a processor 110, a memory 120, a storage device 130, and an input/output device 140. Each of the components 110, 120, 130, and 140 are interconnected using a system bus 150. The processor 110 is capable of processing instructions for execution within the system 100. The processor may be designed using any of a number of architectures. For example, the processor 110 may be a CISC (Complex Instruction Set Computers) processor, a RISC (Reduced Instruction Set Computer) processor, or a MISC (Minimal Instruction Set Computer) processor.

In one embodiment, the processor 110 is a single-threaded processor. In another embodiment, the processor 110 is a multi-threaded processor. The processor 110 is capable of processing instructions stored in the memory 120 or on the storage device 130 to display graphical information for a user interface on the input/output device 140.

The memory 120 stores information within the system 100. In one embodiment, the memory 120 is a computer-readable medium. In one embodiment, the memory 120 is a volatile memory unit. In another embodiment, the memory 120 is a non-volatile memory unit.

The storage device 130 is capable of providing mass storage for the system 100. In one embodiment, the storage device 130 is a computer-readable medium. In various different embodiments, the storage device 130 may be a floppy disk device, a hard disk device, an optical disk device, or a tape device.

The input/output device 140 provides input/output operations for the system 100. In one embodiment, the input/output device 140 includes a keyboard and/or pointing device. In another embodiment, the input/output device 140 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described embodiments by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

Once the 3-D structure of a compound that binds to or interacts with one or more residues of region I or II of the 1IFS structure has been established using any of the above methods, a compound that has substantially the same 3-D structure (or contains a domain that has substantially the same structure) as the identified compound can be made. In this context, “has substantially the same 3-D structure” means that the compound possesses a hydrogen bonding and hydrophobic character that is similar to the identified compound. In some cases, a compound having substantially the same 3-D structure as the identified compound can include a hydroxyl or alkyl moiety.

With the above described 3-D structural data in hand and knowing the chemical structure (e.g., amino acid sequence in the case of a protein) of the region of interest, those of skill in the art would know how to make compounds with the above-described properties. Moreover, one having ordinary skill in the art would know how to derivatize such compounds. Such methods include chemical synthetic methods and, in the case of proteins, recombinant methods.

While not essential, computer-based methods can be used to design the compounds of the invention. Appropriate computer programs include: InsightII (Accelrys), CATALYST (Accelrys), LUDI (Accelrys., San Diego, Calif.), Aladdin (Daylight Chemical Information Systems, Irvine, Calif.); and LEGEND [Nishibata et al. (1985) J. Med. Chem. 36(20):2921-2928], as well as the methods described in the Examples below and the references cited therein.

The above methods can be used to identify small-molecule inhibitors of other type-II ribosome inhibiting proteins. In such embodiments, equivalent residues of the active site as described above could be utilized.

### EXAMPLES

### Example 1

### Identification of Small-Molecule Inhibitors of Ricin Using Virtual Screening and Visual Inspection

Two-stage docking of 236,925 small molecules into the active site of RTA was carried out by the EUDOC program performed on a dedicated cluster of 800 Intel Xeon P4 processors (2.2/2.4 Ghz) according to a published protocol (see, Pang, Y.-P. et al., J. Comput. Chem. 22: 1720-1771 (2001)). The translational and rotational increments at the first stage were 1.0 Å and 10 degrees of arc, respectively, and default increments were used at the second stage. A cutoff of −30 kcal/mol for intermolecular interaction energies was used. The 236,925 small molecules were selected from an in-house database of 2.5 million small molecules using the criterion that each selected molecule has a molecular weight less than 301. All small molecules to be screened were protonated or deprotonated according to physiological pH of 7.4 and their three-dimensional structures and atomic charges were obtained from AM1 semi-empirical calculations. Conformations of RTA and small molecules were not allowed to change during docking. A docking box (6.0×3.5×6.0 Å³) was defined to confine the translation of the mass centre of each molecule within the active site of RTA crystal structure (PDB code: 1IFS). The box was surrounded by Asp100, Ile-104, Asp75, Asn78, Tyr80, Val82, Phe93, Gly120, Gly121, Asn122, His94, Pro95, and Asp96 whose conformations are as defined in the 1IFS crystal structure (region I) or by Tyr80, Val81, Phe93, Gly121, Asn122, Tyr123, Ile172, Arg180, Ala79, Ser176, Glu177, and Leu126 whose conformations were defined in the 1IFS crystal structure (region II). All water molecules and the bound adenine were removed from the RTA crystal structure. Arg213 of RTA adopts the conformation uniquely defined in the 1IFS crystal structure. Compounds provided herein had a EUDOC energy of <−54 kcal/mol obtained from a virtual screening study. Compounds showing in vitro anti-ricin activities were further derivatized, through visual inspection of the EUDOC-generated inhibitor-RTA complexes, by adding functional groups such as a hydroxyl or alkyl group to improve their intermolecular interactions.

### Example 2

### Testing Small-Molecule Inhibitors of Ricin Using a Cell-Free In Vitro Translation Assay with a Luciferase Reporter

Compounds I-1, IV-5, IV-7, III-1, VII-2, I-3, I-5, II-3, II-13, VII-13, IV-10, II-2, IV-3, II-12, VII-3, V-21, IV-9, VII-1, V-1, IV-19, I-4 and IV-1 were tested for inhibition of ricin using a cell-free in vitro translation assay as described previously and as further described below. A yeast cell-free translation-competent extract was prepared in the lab based on the method presented by Iizuka and Sarnow (see, Iizuka, N. and Sarnow, P., Methods: A Companion to Methods in Enzymology 11(4):353-360 (1997)). W303 yeast (killer virus minus strain) cells were grown in liquid culture media (YPD). Cells were spun down and washed three times in WCE-Mannitol. Yeast cells were broken open by vortexing in the presence of glass beads and WCE-PMSF-Mannitol. Small inhibitory compounds were removed by chromatography (Sephadex™ G-25). The 60-90 OD₂₆₀ fractions were collected from the column and this represents the yeast cell-free extract used in the translation assay. Capped luciferase RNA was produced using the Epicenter AmpliCap T7 Kit (AC0707) and was added to the yeast extract to provide the RNA template for translation. Each compound was prepared in 100% DMSO. Working solutions of the compounds (3 mM) were then prepared at a final DMSO concentration of 10%. The final DMSO concentration for every in vitro translation reaction was standardized to 0.67% DMSO. RTA was incubated with the compound (equimolar concentration, 20 nM) on ice for 20 min. prior to addition to the in vitro translation mixture. As a negative control, cycloheximide was used as a translation inhibitor in the in vitro assay instead of ricin, to identify small molecules that may affect other steps in translation (other than ribosome depurination).

The reaction was run at 23° C. for 1 hour. The assay has been successfully carried out in the 96-well format using 15 or 30 μL volumes per well. Following the 1 hour incubation, the reaction was stopped by the addition of 100 μL TBS buffer. White 96 microwell plates (Nunc 236105) were used to setup the luminometer assay. Briefly, 20 μL of the diluted translation assay mixture was added to a well of the plate. The amount of active luciferase protein (indicating translation efficiency of the in vitro reaction) is measured using the Biotek 96 well-plate luminometer. The system was set up such that the automatic injector added 100 μL of Promega's luciferase assay reagent (LAR) to each well (2 second delay, 10 second integrated light measurement).

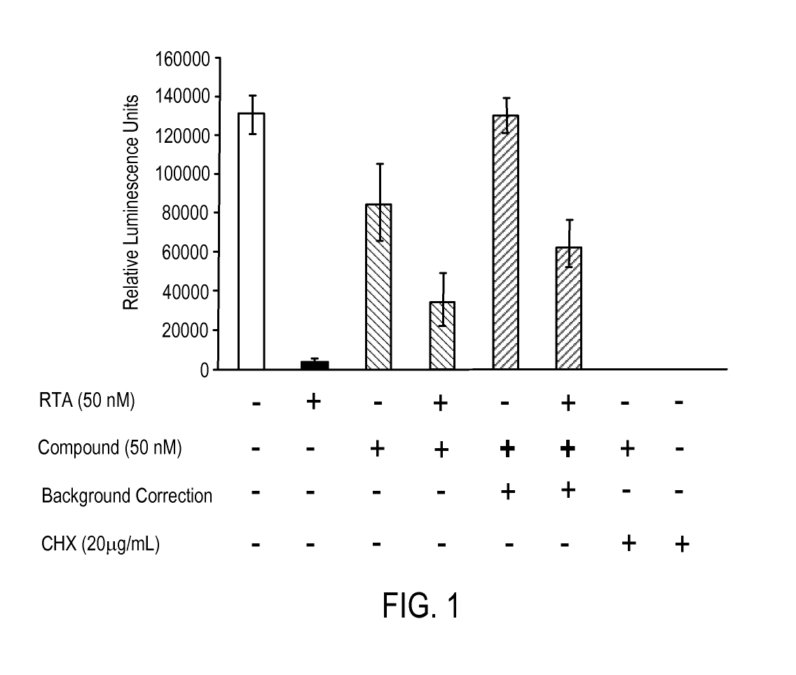

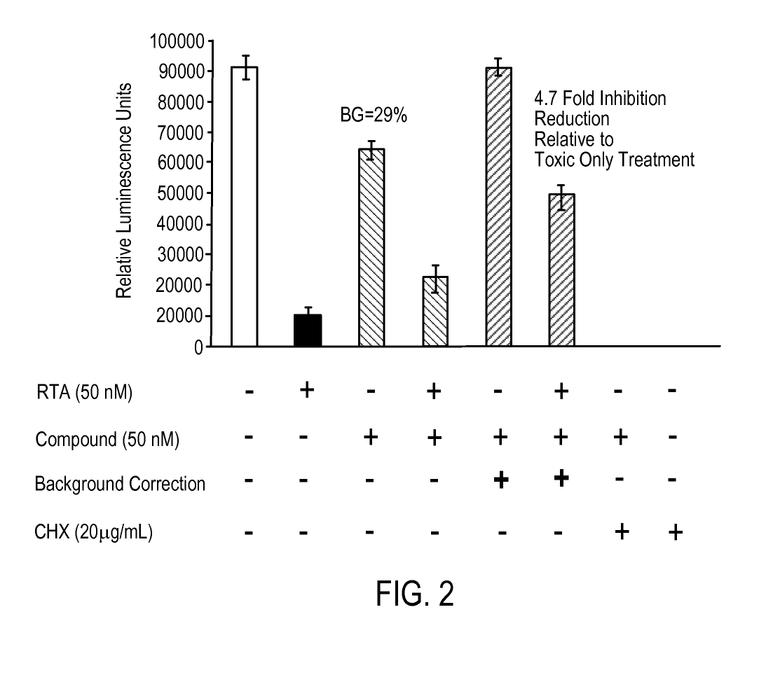

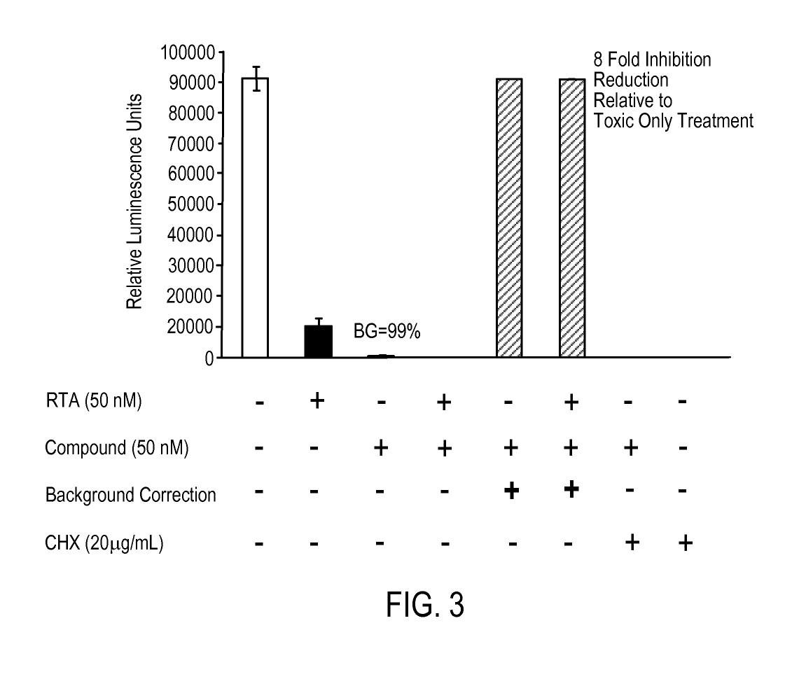

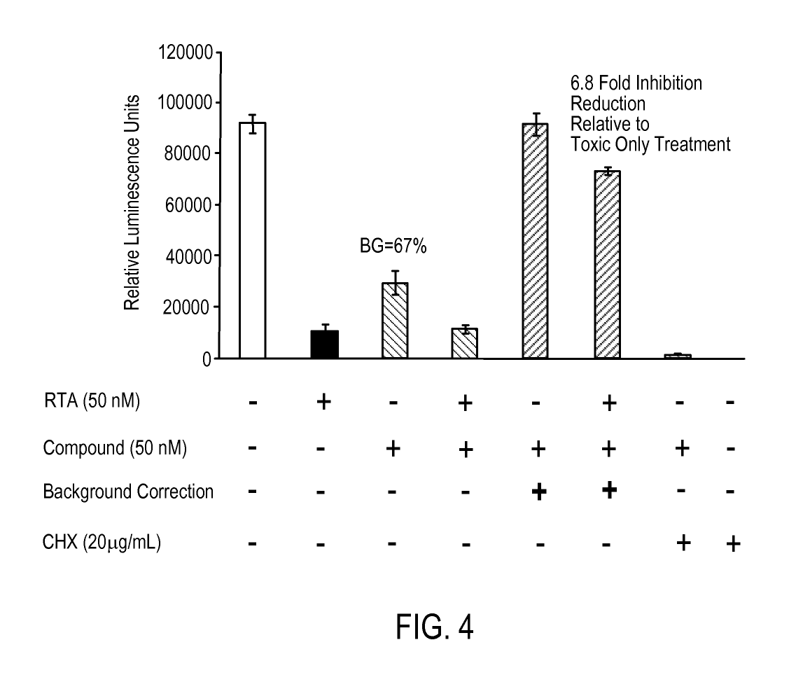

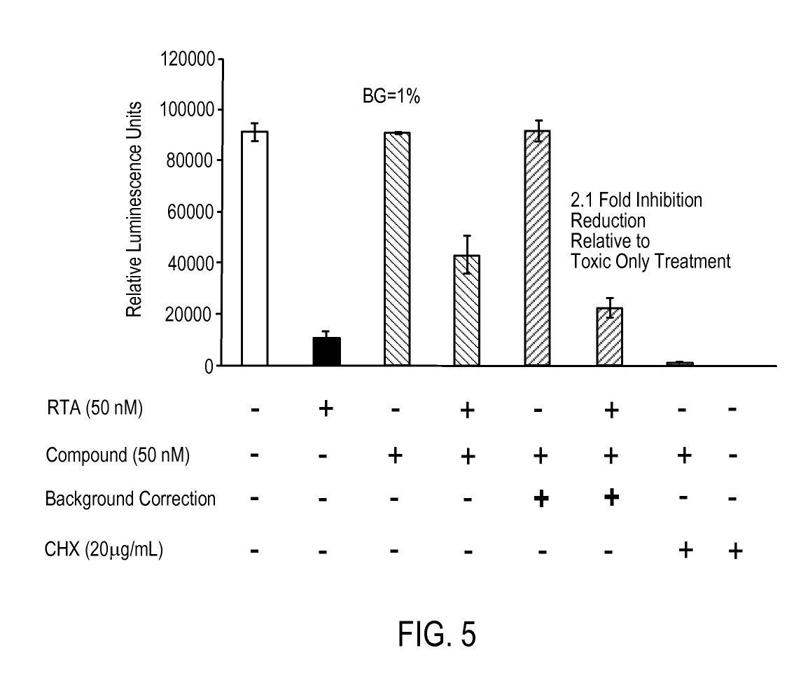

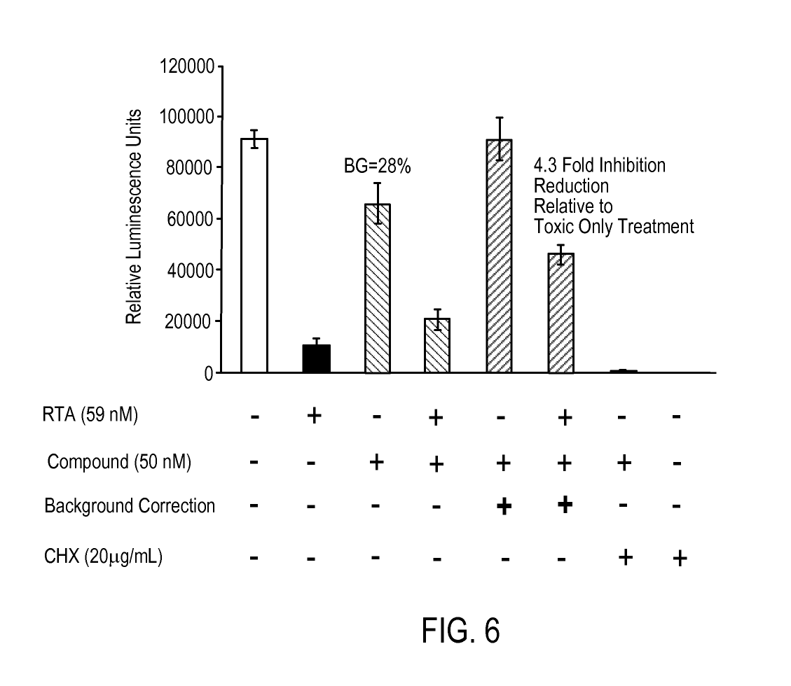

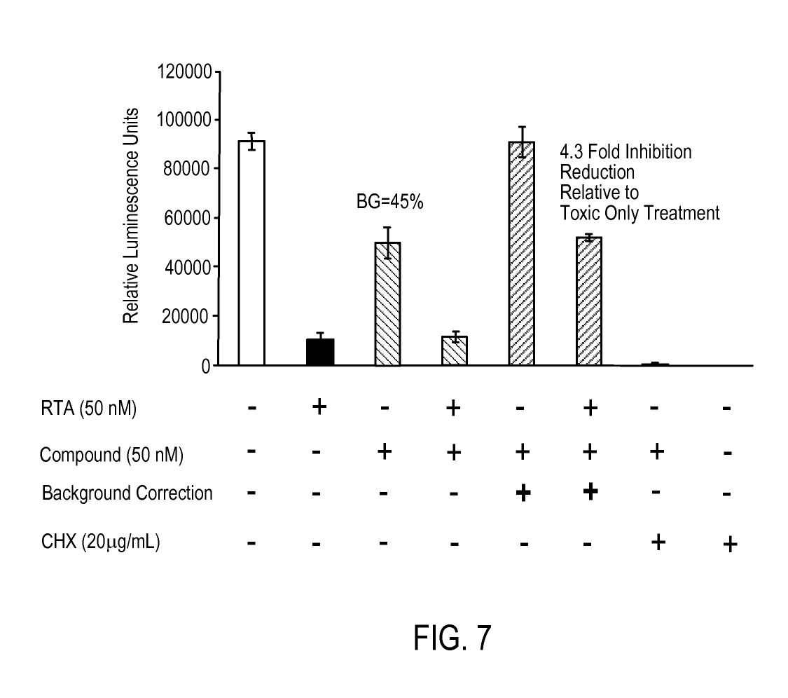

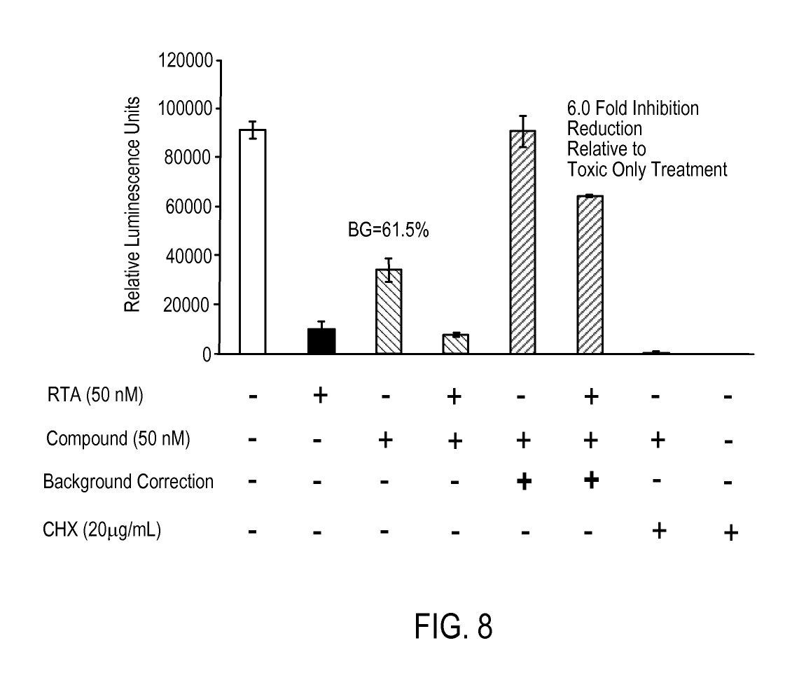

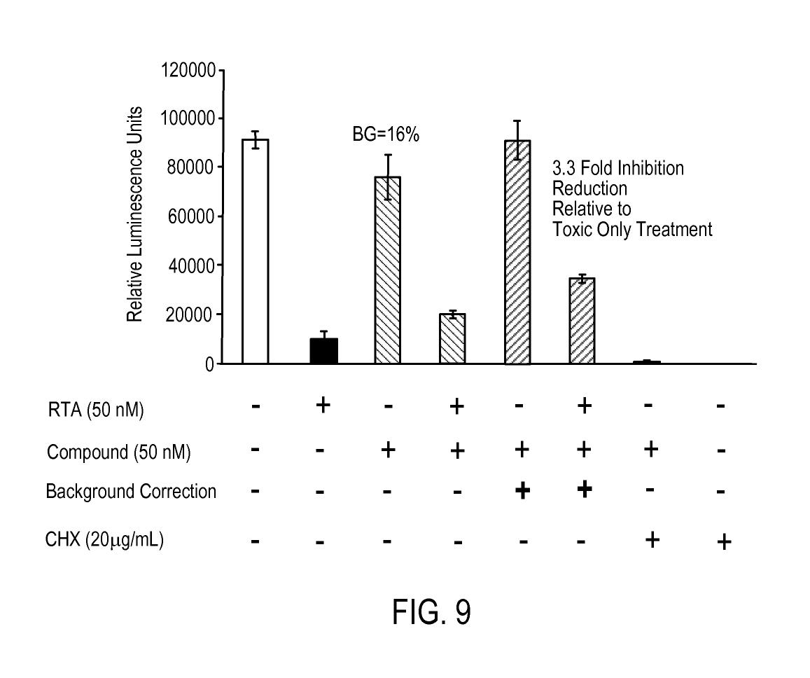

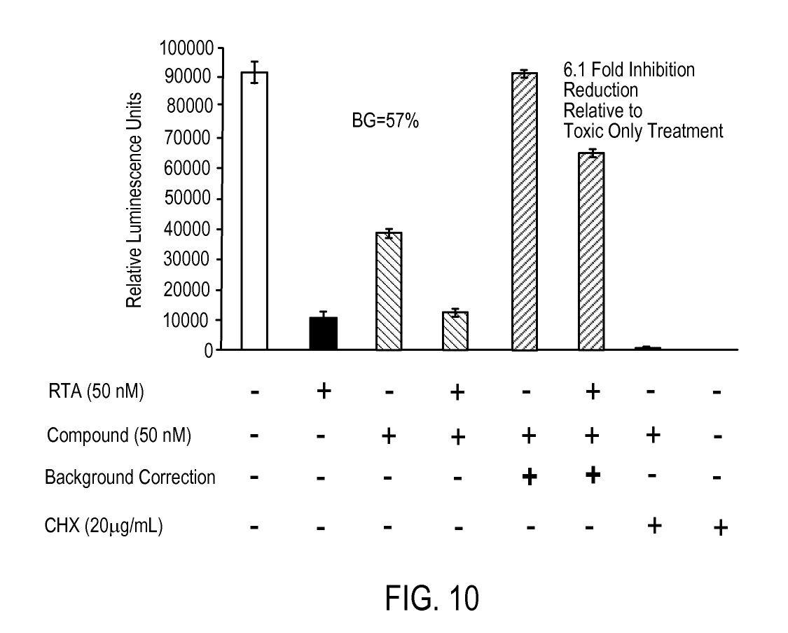

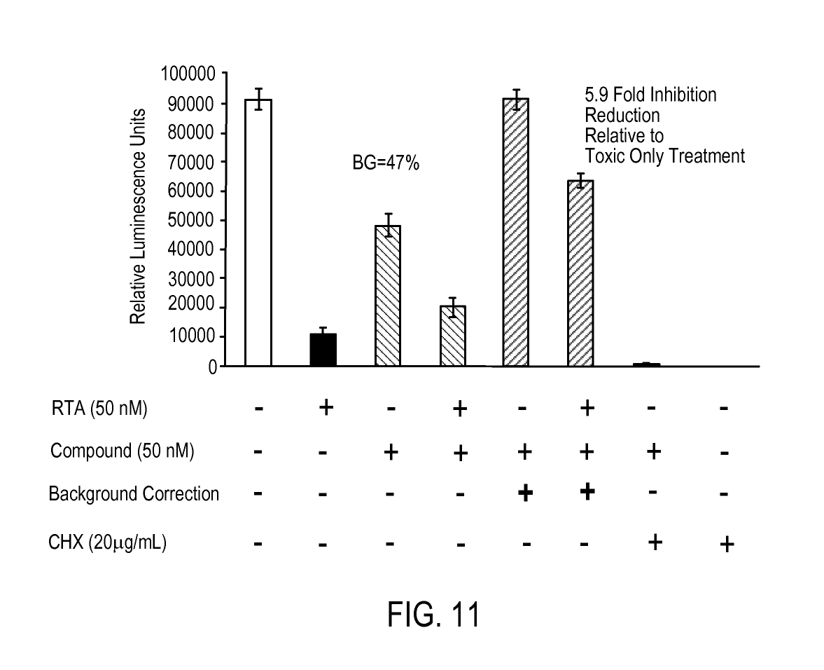

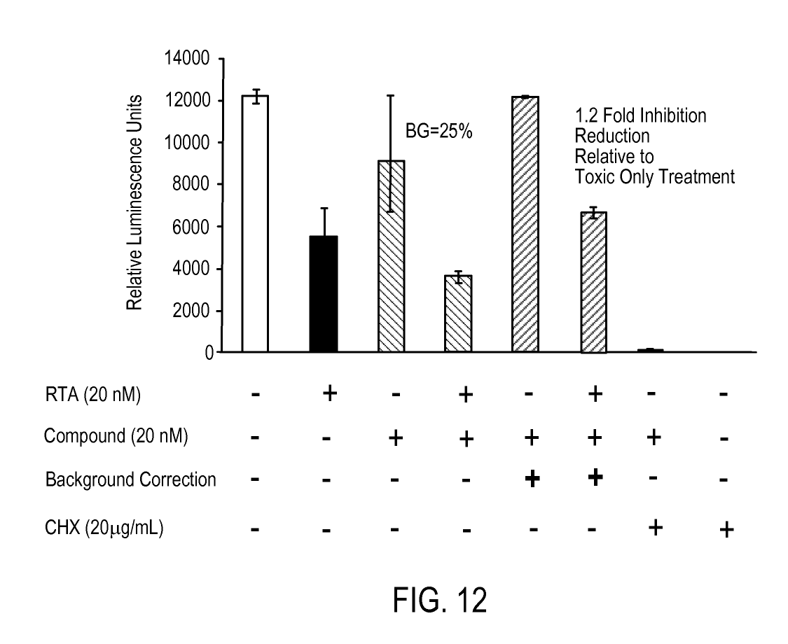

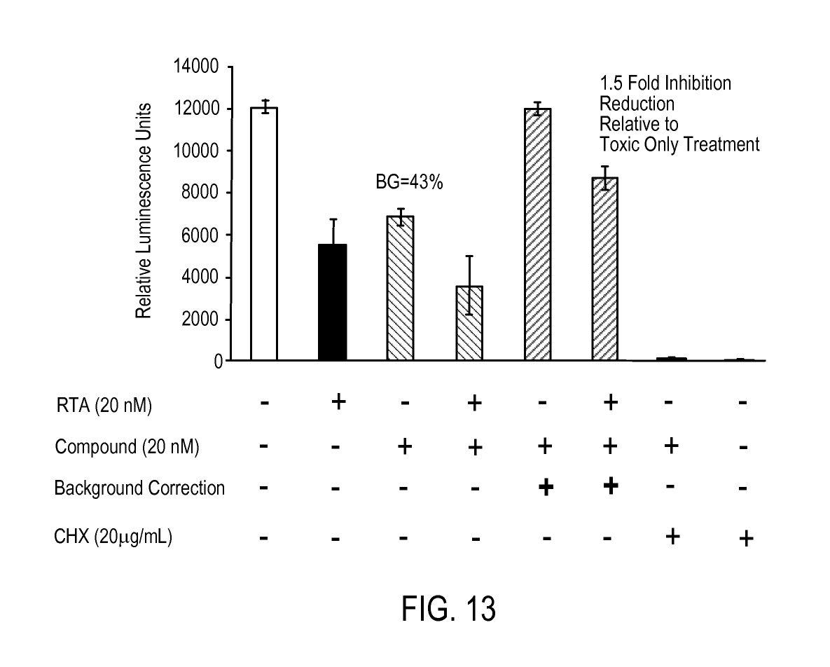

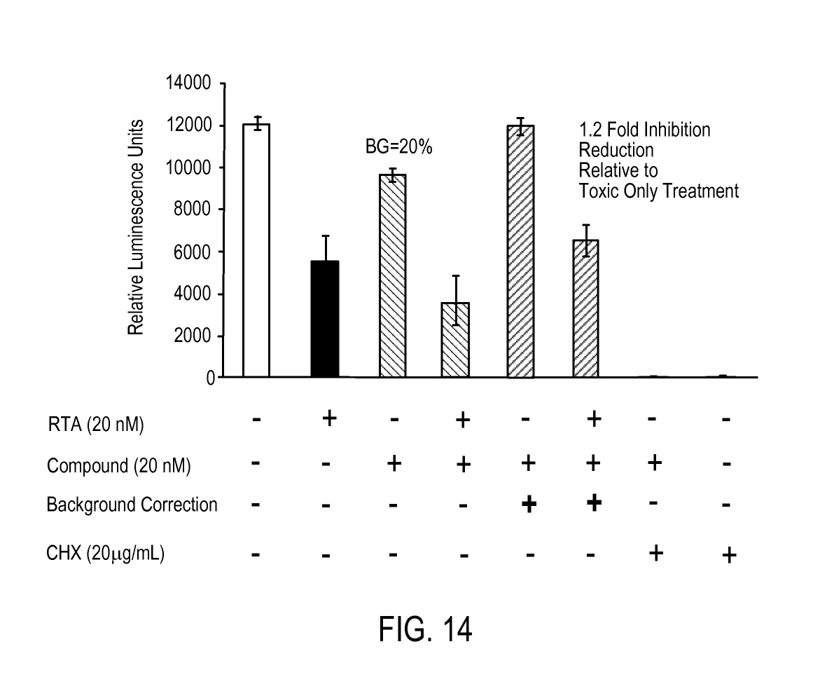

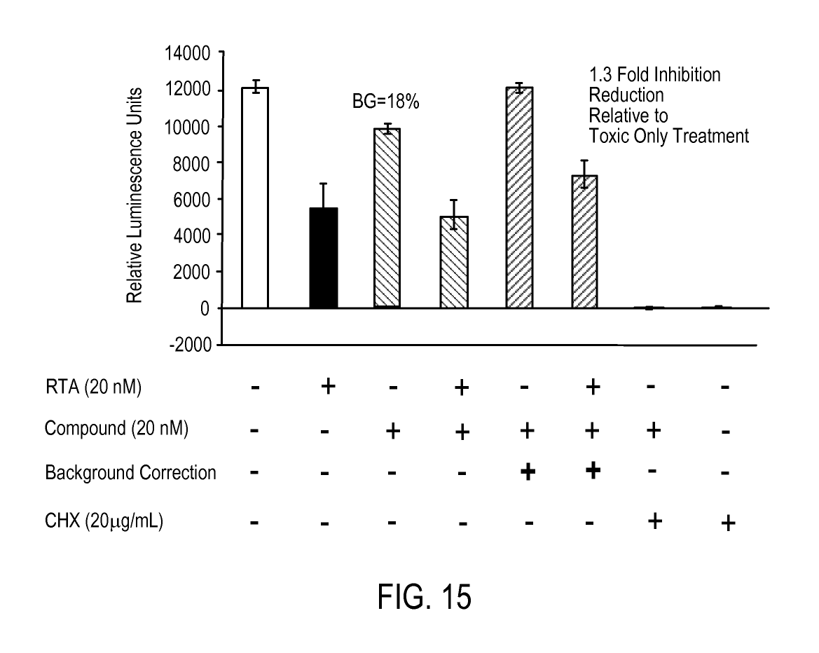

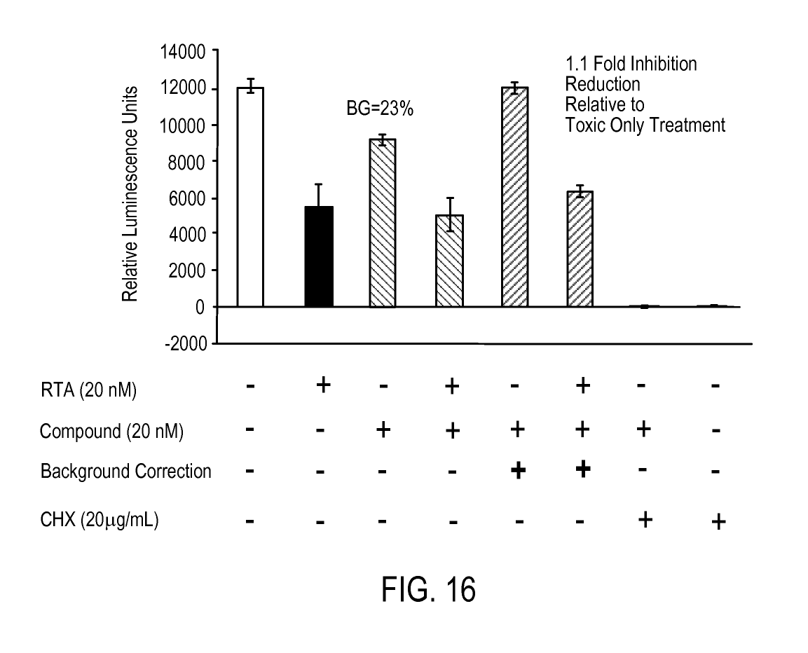

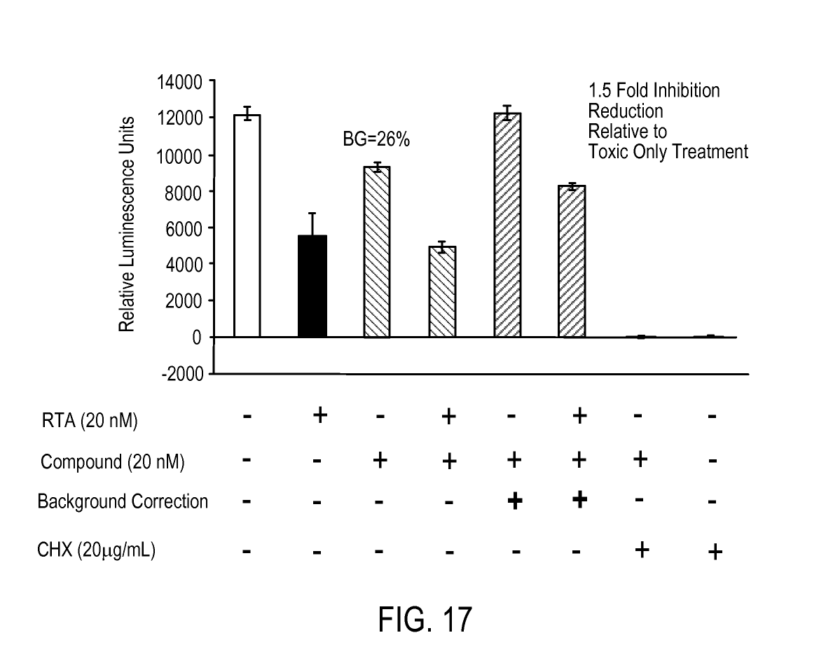

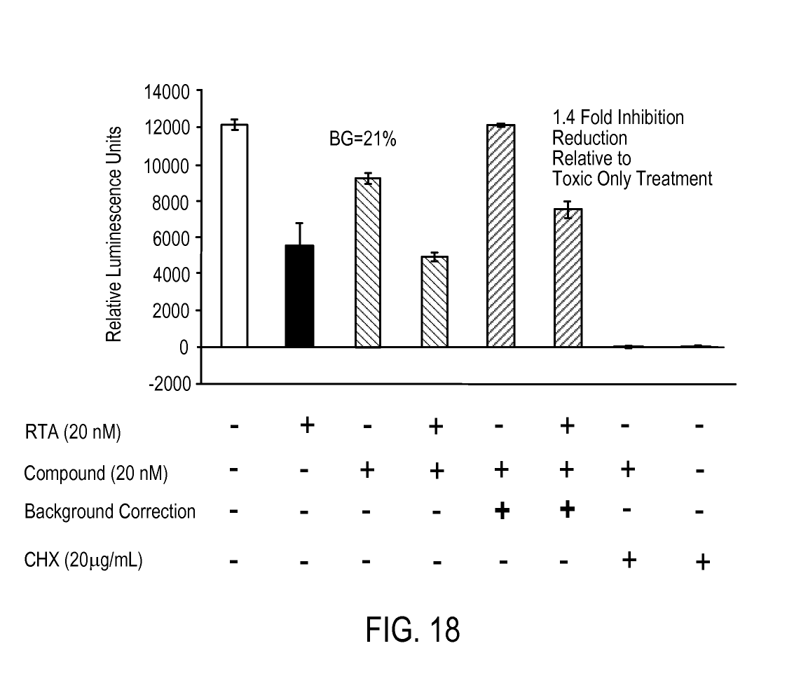

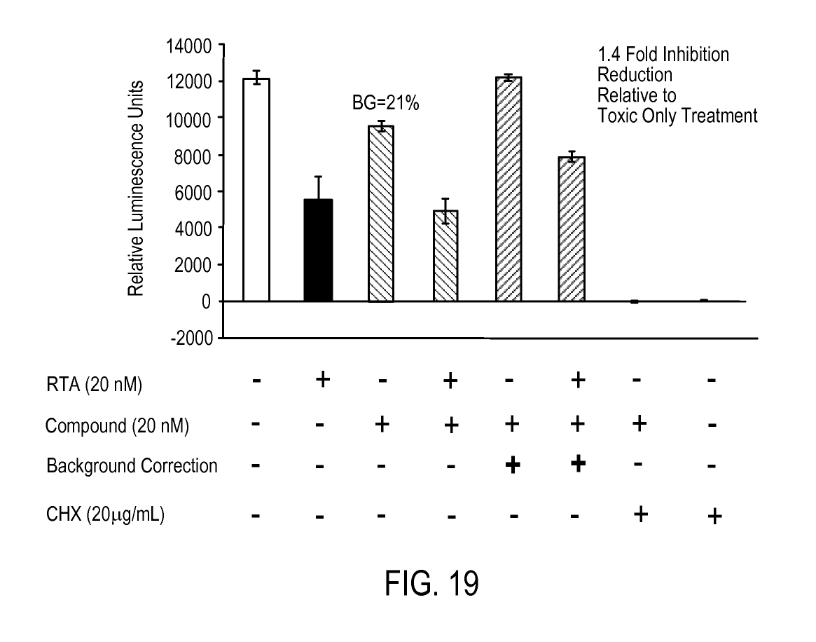

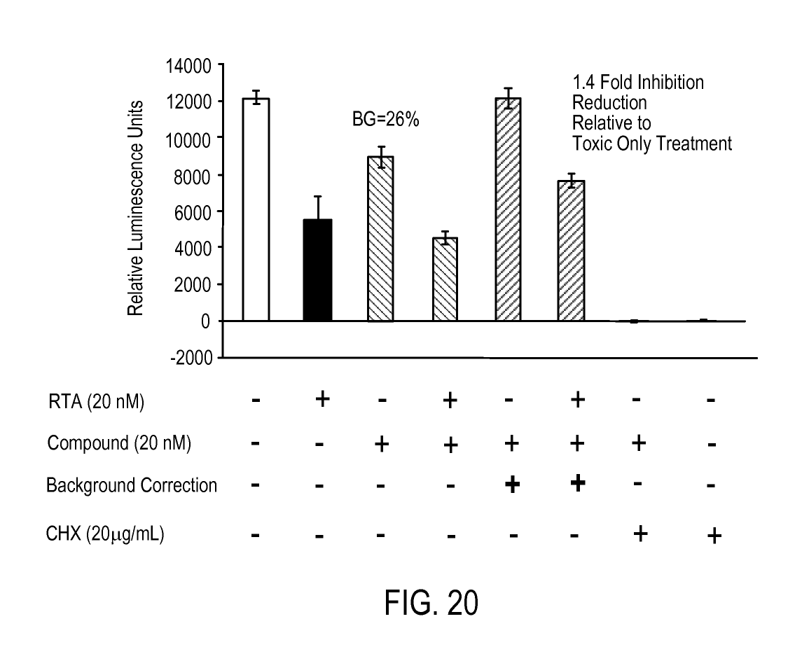

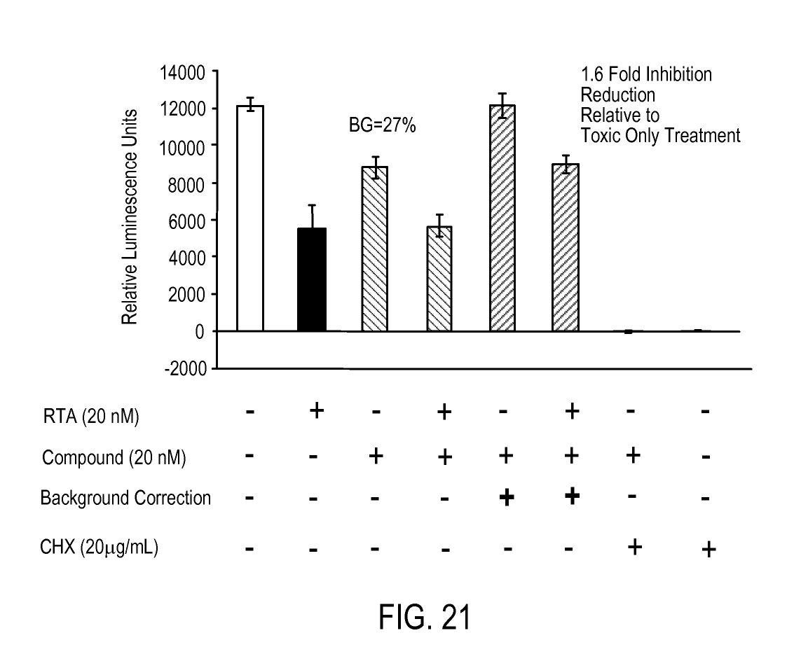

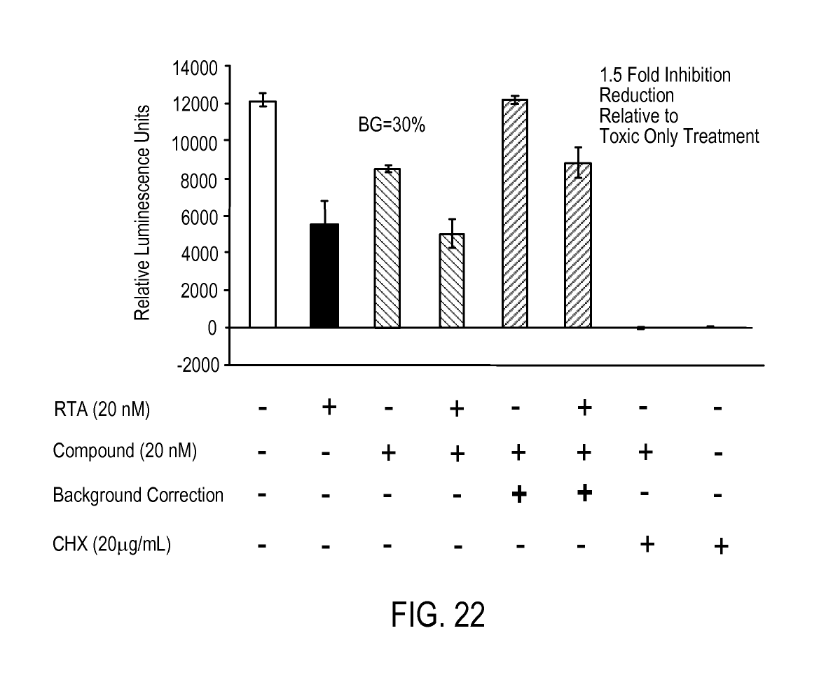

FIG. 1 presents the in vitro translation assay results for compound I-1. This compound had a moderate effect on the background (35% reduction relative to the control) and a large effect on inhibition of RTA (15.2 fold decrease in inhibition relative to the toxin only treatment). The data are presented both with and without background correction (blue=raw data, red=adjusted data). FIGS. 2-11 represent results for compounds IV-5, IV-7, III-1, VII-2, I-3, I-5, II-3, II-13, VII-13, and IV-10, respectively (20 nM equimolar concentration test). FIGS. 12-22 represents results for compounds II-2, IV-3, II-12, VII-3, V-21, IV-9, VII-1, V-1, IV-19, I-4 and IV-1 (20 nM equimolar concentration test). According to the in vitro translation assay, the IC₅₀ values of IV-3, V-21, IV-9, and IV-8 for inhibiting RTA were estimated to be 10 nM, 100 nM, 1 nM, and 0.1 nM, respectively.

### Example 3

### Testing Small-Molecule Inhibitors of Shiga-Like Toxin Using a Cell-Free in Vitro Translation Assay with a Luciferase Reporter

An in vitro cell-free bioassay was developed to screen for small-molecules that inhibit the enzymatic activity of Shiga toxin 2. The bioassay is based on a rabbit reticulocyte cell-free lysate (RRL) system. The in vitro screen can be used to determine if the inhibitor acts at the level of translation. The following describes the in vitro cell-free translation assay. The assay was applied to compounds I-1, I-2, IV-5, and IV-6.

The RRL assay was developed using 2:1 RRL obtained from Green Hectares (Oregon, Wis.). The RRL was supplemented with the same ATP regeneration system used for the yeast in vitro translation assays (Iizuka and Sarnow, 1997). The assay was performed identically as in Example 2 except the reaction is incubated at 30° C. for 1 hour. Uncapped luciferase RNA was produced using the Epicenter AmpliScribe T7 kit (AS3107) and was added to the RRL to provide the RNA template for translation. The test compounds were prepared in 100% DMSO. Working solutions of the compounds (3 mM) were then prepared at a final DMSO concentration of 10%. The final DMSO concentration for every in vitro translation reaction was standardized to 0.67% DMSO. Shiga-like toxin 2 (Stx2) was incubated with the compound (equimolar concentration, 10 nM) on ice for 20 min. prior to addition to the in vitro translation mixture. As a negative control, cycloheximide was used as a translation inhibitor in the in vitro assay instead of Stx2, to identify small molecules that may affect other steps in translation (other than ribosome depurination).

The reaction was run at 30° C. for 1 hour. The assay was successfully carried out in the 96-well format using 15 or 30 μL volumes per well. Following the 1 hour incubation, the reaction was stopped by the addition of 100 μL TBS buffer. White 96 microwell plates (Nunc 236105) were used to setup the luminometer assay. Briefly, 20 μL of the diluted translation assay mixture was added to a well of the plate. The amount of active luciferase protein (indicating translation efficiency of the in vitro reaction) was measured using the Biotek 96 well-plate luminometer. The system was set up such that the automatic injector added 100 μL of Promega's luciferase assay reagent (LAR) to each well (2 second delay, 10 second integrated light measurement).

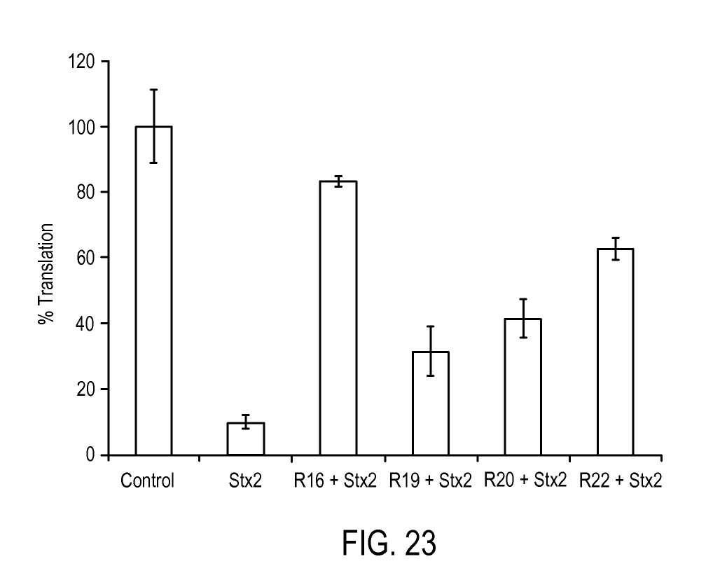

According to the above-described in vitro translation assay, IV-3, V-21, IV-9, and IV-8 showed a 18-, 14-, 9- and 7-fold decrease of the inhibition caused by Stx2 relative to the toxin only treatment at a drug concentration of 10 nM (FIG. 23).

### Example 4

### Testing Small-Molecule Inhibitors of Ricin Using a Colorimetric-Based Mouse Myeloma Cell Viability Assay

RTA antagonists were tested at three different concentrations (0.3, 3, or 30 μM) under three different types of assays: (a) cells+RTA inhibitors+ricin mixed together at the same time (neutralization); (b) cells+RTA inhibitors preincubated before ricin challenge (pre-treat); (c) cells+ricin preincubated for some time before adding ricin inhibitors (rescue). The antagonists were incubated with 1e4 Sp2/0-Ag14 (Sp2) mouse myeloma cells in hybridoma serum-free medium for 3 hrs at 37° C. in 96-well microplates. Ricin was added to the cells to yield 40 pg/mL final concentration and the mixtures were further incubated overnight. Metabolic activity of the cells were determined using the CellTiter 96 Aqueous Cell Proliferation Assay (Promega). The results are expressed in percent of the metabolic activity of Sp2 cells incubated under the same conditions in the absence of ricin and RTA antagonists. All experiments were made in six parallels.

Specifically, mouse myeloma Sp2/0-Ag14 (CRL-1581, American Type Culture Collection, Manassas, Va.) cells were pre-grown to early-mid log phase in Hybridoma Serum Free Medium (HSFM, Invitrogen, Carlsbad, Calif.) supplemented with 4 mM Glutamax (Invitrogen), 0.5% (v/v) penicillin and streptomycin mix (Invitrogen). Cells were collected with low-speed centrifugation (1,500 rpm in a Sorvall RT-6000 centrifuge, Thermo Electron Corp., Ashville, N.C.) at 4° C. for 15 minutes, resuspended in fresh HSFM and plated in the wells of 96-well sterile microplates (Costar 3595) to result in 2.5e+5/mL final cell density. The cells were incubated in the absence of any other additives (Viability Control), in the presence of 50 pg/mL ricin (Vector, Burlingame, Calif.) (Ricin Inhibition Control), in the presence of the test substance (30, 3 and 0.3 μM) (Substance Toxicity Control) and in the combined presence of the above amounts of ricin and test substances (Test) in 5% CO₂ atmosphere with 100% relative humidity at 37° C. for 18 hours. MTS/PMS from the Cell Titer 96 AQuaeous Non-Radioactive Cell Proliferation Assay (Promega, Madison, Wis.) mix was added to the cells according to the manufacturer's recommendations and the plates were read at 490 nm after further incubation for 4 hours. The data was transformed by subtracting the OD490 data obtained with the Ricin Inhibition Control from all OD490 values where ricin was present. Cell viabilities in at least 3 parallel wells containing the mixtures of ricin and tests substances were calculated by expressing the OD490 values in percent of the OD490 values of at least 3 parallel wells of Viability Control (% Viability). A positive % Viability value, exceeding the intra-assay variance obtained with the Ricin Inhibition Control was taken as the indication of the ricin antagonist effect of the test substance. A % Viability value in the Substance Toxicity Control less than the value obtained in Viability Control was a direct measure of the cell toxic nature of the test substance. A negative value in Test was also indicative of the toxicity of the substance. If this negative value was not coupled with a decreased % Viability in Substance Toxicity Control, then the substance was toxic only in the presence of ricin.

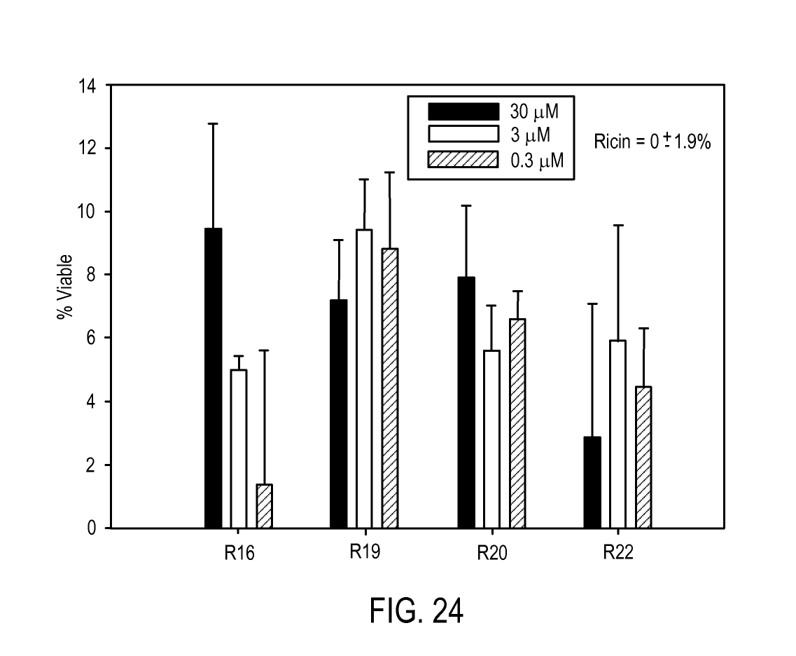

The results show that compounds present in the top 10 for anti-ricin activity in all three assay types (neutralization, pre-treat and rescue) included: V-1, IV-9, and IV-3. IV-7 was in the top 10 for activity in two assay types (neutralization and rescue). The following substances were among the top 10 for activity in a single assay type: IV-8 (neutralization), V-21 (pre-treat) and IV-1 (rescue). IV-3, V-21, IV-9, and IV-8 showed 1.4, 8.8, 6.6, and 4.4% cell protection against ricin at a drug concentration of 300 nM, respectively (FIG. 24).

### Example 5

### Testing Small-Molecule Inhibitors of Stx2 Using a Colorimetric-Based Vero Cell Viability Assay

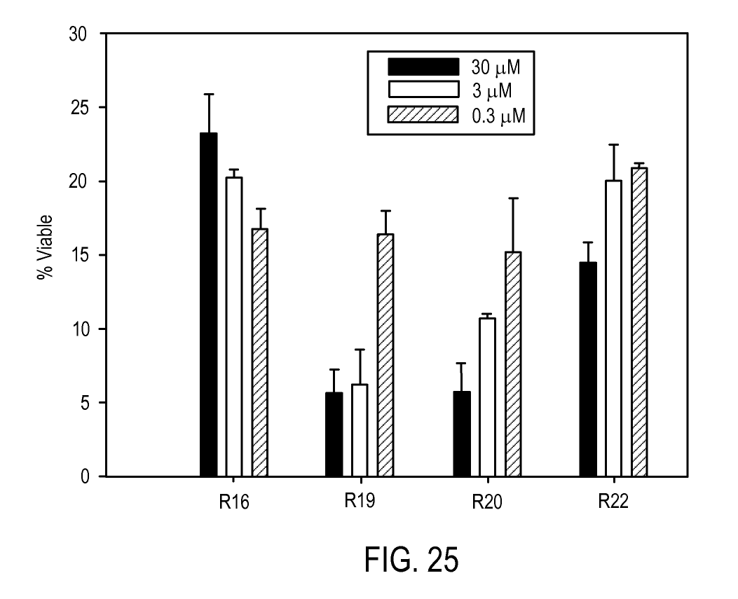

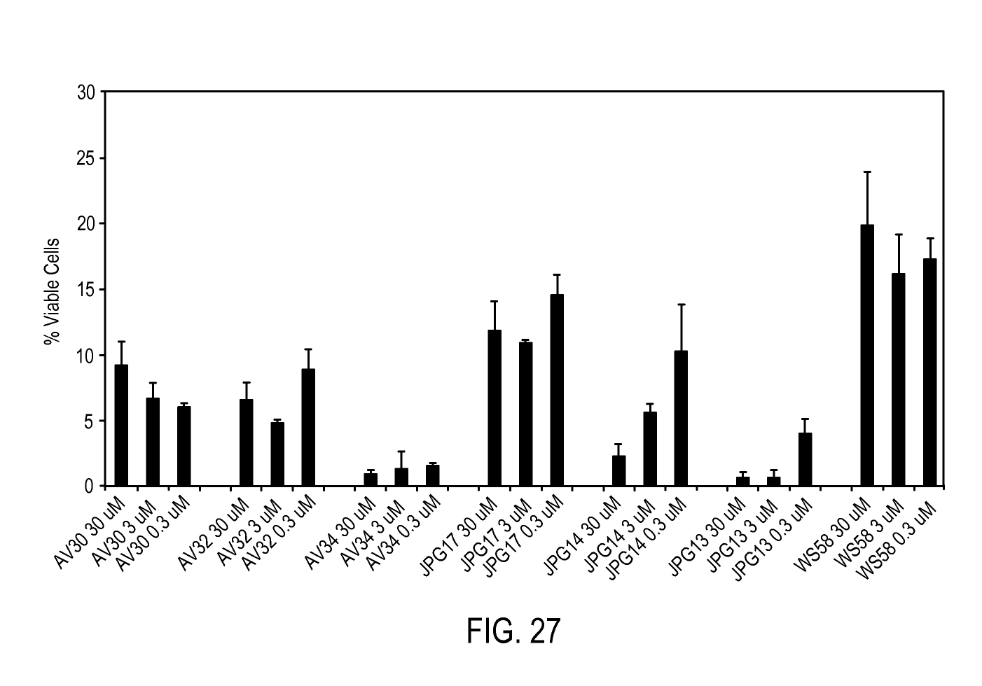

Using the same assay described in Example 4 except that Vero cells (ATCC CCL-81) replaced Sp2/0-Ag14, IV-3, V-21, IV-9, IV-61, IV-59 and IV-8 showed 15 to 20% cell protection against Stx2 at drug concentration of 300 nM (see FIGS. 25 and 27).

### Example 6

### Solution and Solid-Phase Syntheses of Compounds of Formula IV-A

Compounds according to Formula IV-A can be prepared by solution and solid-phase syntheses as exemplified in Schemes 1 and 2 below.

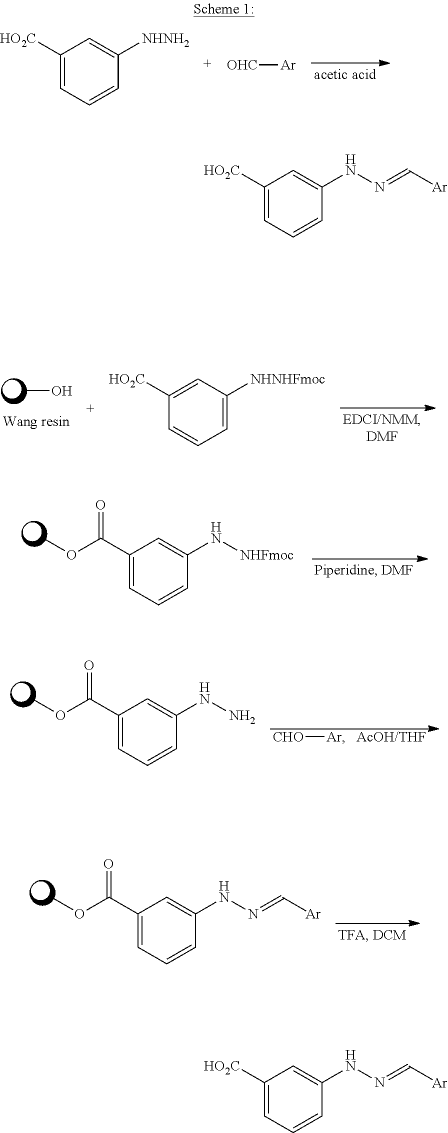

Examples of commercially available OHC—Ar are listed below:

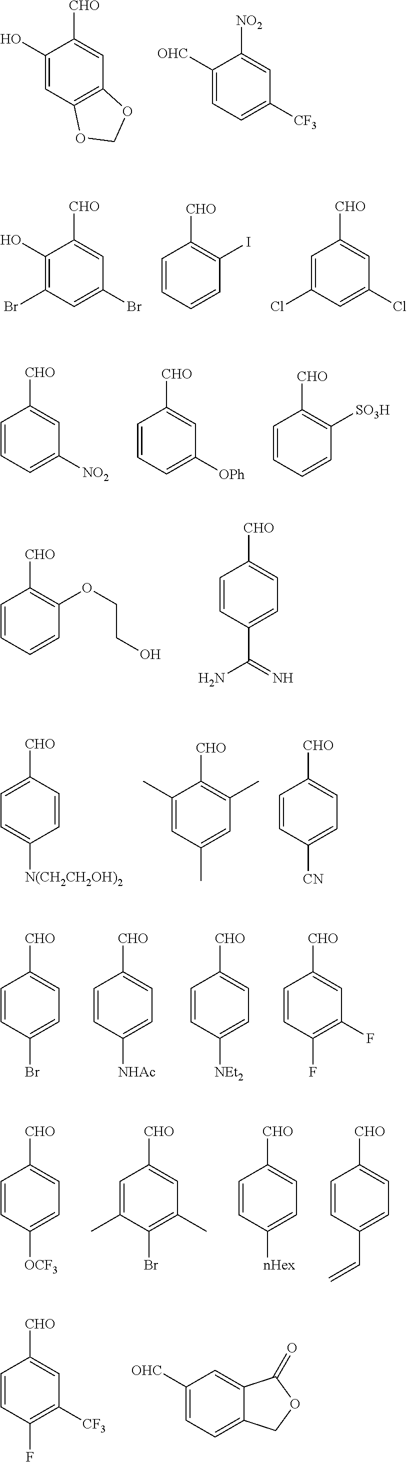

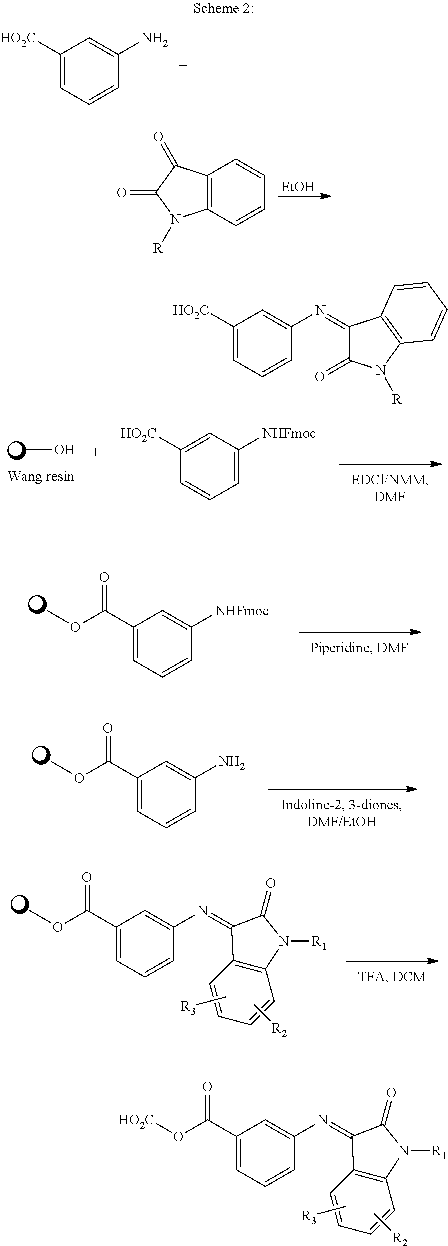

Examples of commercially available indoline-2,3-diones are listed below:

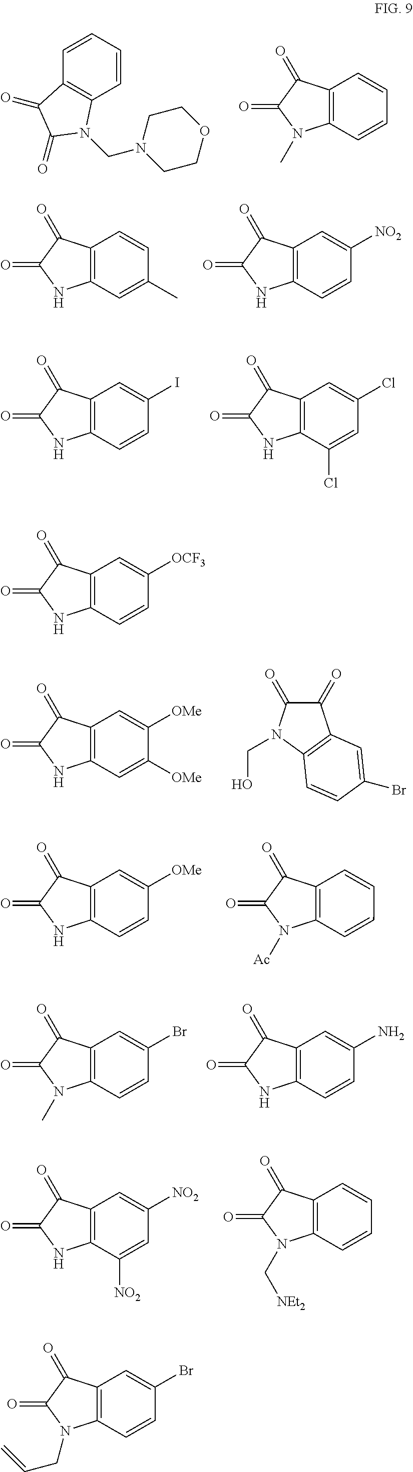

### Example 7

### Solution and Solid-Phase Syntheses of Compounds of Formula V-A

Compounds according to Formula V-A can be prepared by solution and solid-phase syntheses as exemplified in Scheme 3 below.

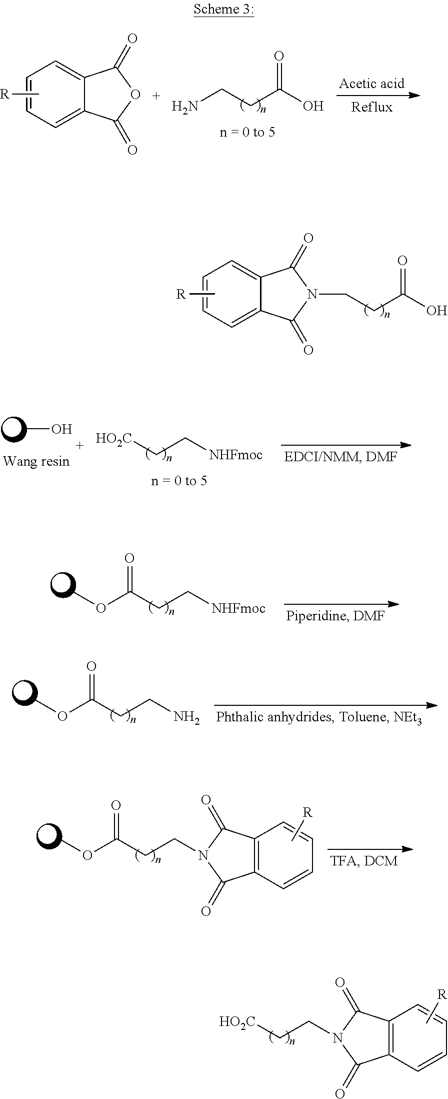

Examples of commercially available phthalic anhydrides are listed below:

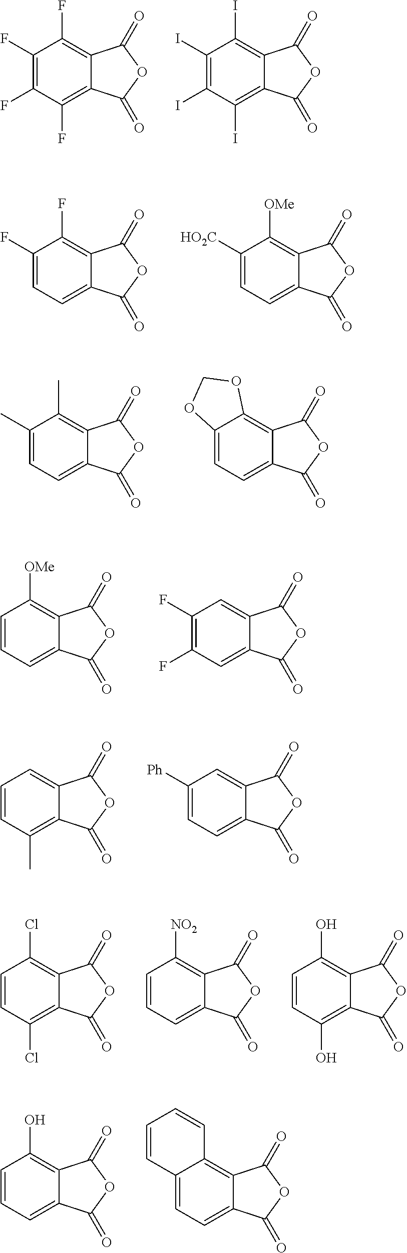

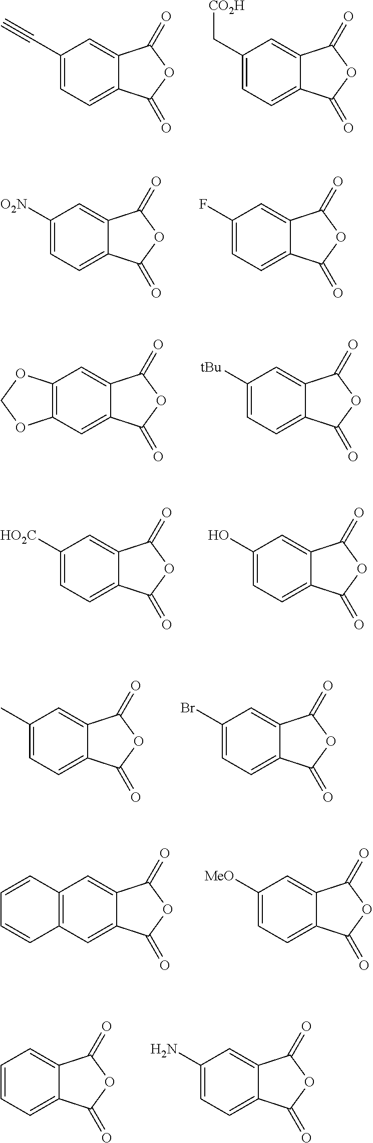

### Example 8

### Solution and Solid-Phase Syntheses of Compounds of Formula VI-A

Compounds according to Formula VI-A can be prepared as shown in Scheme 4. The wavy lines represent the point of attachment for each moiety.

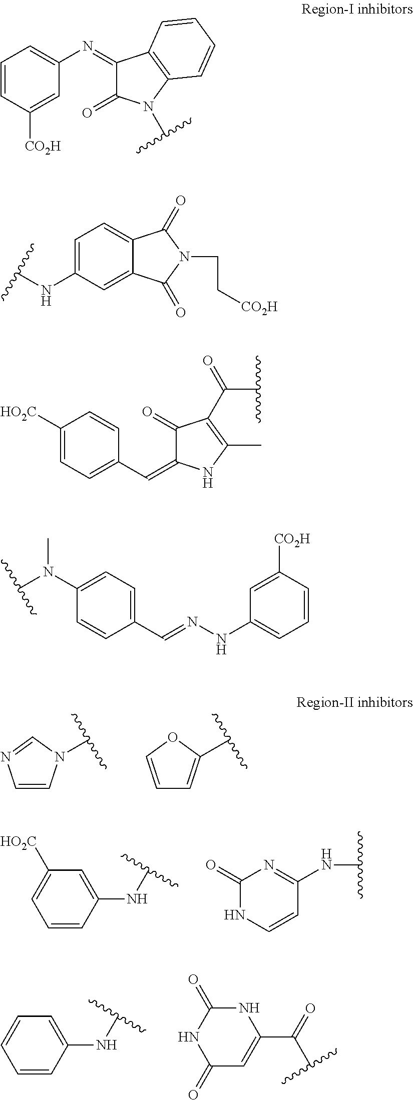

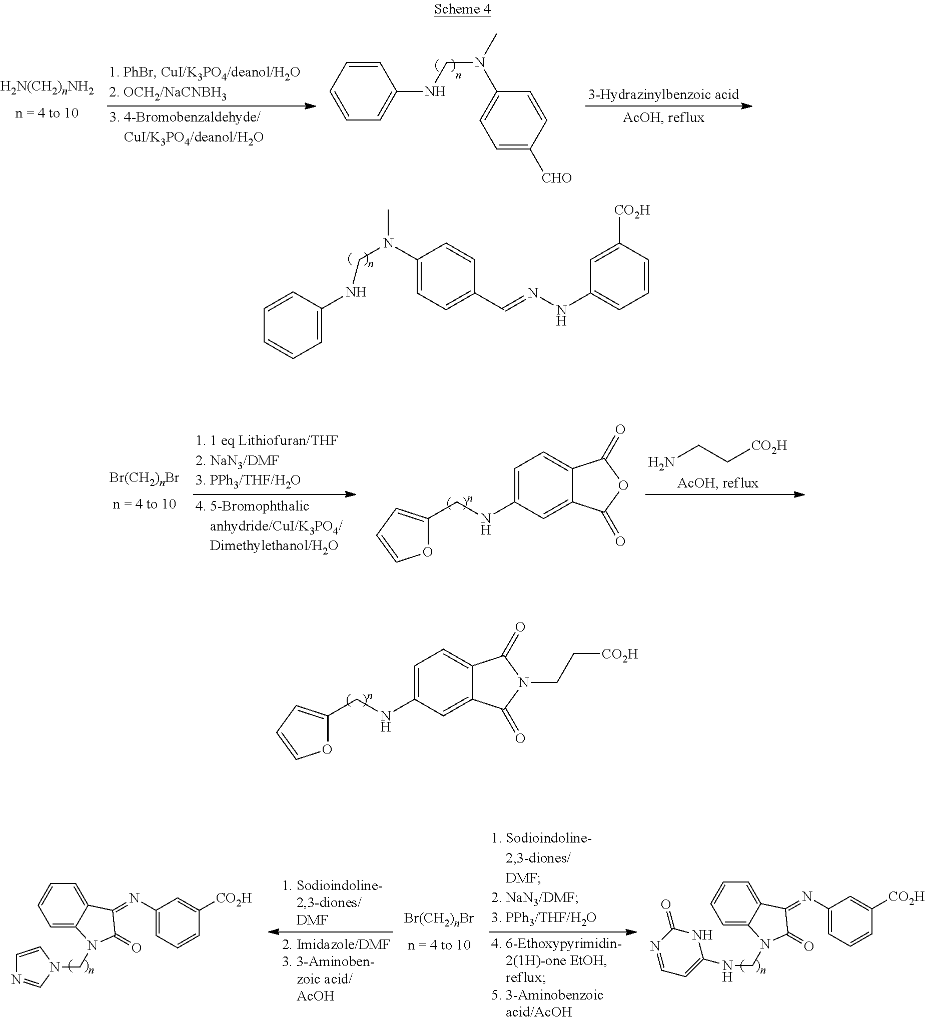

### Example 9

### Comparison of Activity of Selected RTA Inhibitors Ex Vivo

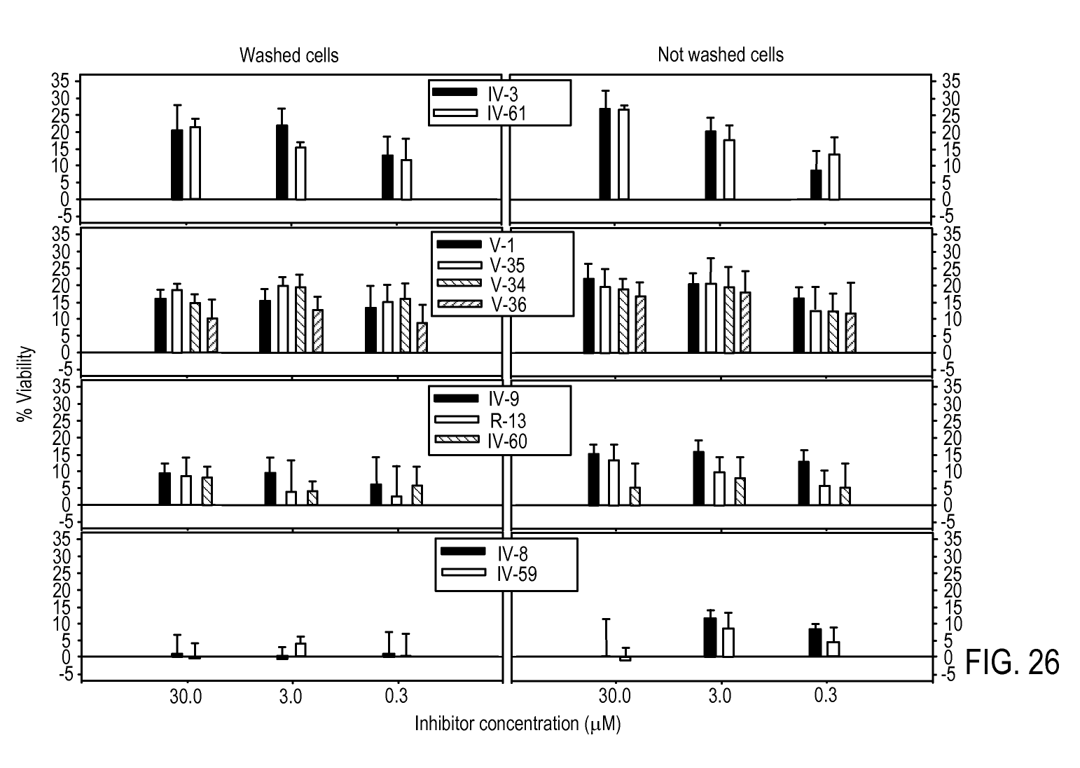

Sp2 mouse myeloma cells were exposed at 37° C. for 2 hours to the different ricin inhibitors detailed in Table 1 at the concentrations shown. Cells were centrifuged and resuspended in inhibitor-free growth medium before adding ricin. The cells were then incubated in the presence of ricin (40 pg/mL) at 37° C. for 16 hours. The metabolic activity of the cells was determined with the CellTiter 96 non-radioactive cell proliferation assay (Promega) and the results were expressed in percent of metabolic activity of similarly treated cells incubated in the absence of ricin. The data in Table 1 shows the means of 8 parallel experiments along with the standard deviation (SD) (shown as vertical bars in FIG. 26). Student's t-test was used to evaluate the differences between the various experiments. In cases in which the numbers did not pass the equal variance test, the Mann-Whitney rank sum test was used to evaluate the differences. Comparisons were made at the concentrations associated with the highest activity of the 2^{nd} group of inhibitors.

TABLE 1
Comparison of activity of 1^{st} and 2^{nd} generation RTA inhibitors ex vivo

| Compound | | | | Compared | |
|---|---|---|---|---|---|
| | % Viable | | % Viable | concentration | |
| 1^{st} group | (SD) | 2^{nd} group | (SD) | (μM) | P |
| IV-3 | 20.7 (7.3) | IV-61 | 21.7 (2.1) | 30 | 0.875 |
| V-1 | 14.8 (3.9) | V-35 | 19.7 (2.3) | 3 | 0.009 |
| | | V-34 | 19.3 (3.6) | | 0.032 |
| | | V-36 | 12.8 (3.5) | | 0.282 |
| IV-9 | 9.4 (2.8) | IV-62 | 8.3 (5.5) | 30 | 0.626 |
| | | IV-60 | 7.8 (3.4) | | 0.340 |
| IV-8 | −1.0 (3.7) | IV-59 | 3.9 (2.1) | 3 | 0.382 |

### Example 10

### Cell Viability with and without Removal of Ricin Inhibitors

Sp2 mouse myeloma cells were exposed at 37° C. for 2 hours to the different ricin inhibitors detailed in Table 2 at the concentrations shown. An aliquot of the cells was centrifuged and resuspended in inhibitor-free growth medium (washed cells) before adding ricin. Another aliquot of the cells received ricin without removing the inhibitor (not washed cells). The cells were incubated in the presence of ricin (40 pg/mL) at 37° C. for 16 hours. The metabolic activity of the cells was determined with the CellTiter 96 non-radioactive cell proliferation assay (Promega) and the results were expressed in percent of metabolic activity of similarly treated cells incubated in the absence of ricin. The data in Table 2 shows the means of 8 parallel experiments along with the standard deviation (SD). Student's t-test was used to evaluate the differences between the various experiments. In cases in which the numbers did not pass the equal variance test, the Mann-Whitney rank sum test was used to evaluate the differences.

TABLE 2
Cell viability with and without removal of ricin inhibitors

% metabolic
activity (SD)

| | Conc. | Not washed | Washed | |
|---|---|---|---|---|
| Compound | (μM) | cells | cells | P |
| IV-3 | 30 | 27.0 (5.3) | 20.7 (7.3) | 0.070 |
| | 3 | 20.1 (4.2) | 22.0 (4.8) | 0.409 |
| | 0.3 | 8.6 (5.7) | 13.1 (5.6) | 0.161 |
| IV-61 | 30 | 26.7 (1.3) | 21.7 (2.1) | <0.001 |
| | 3 | 17.7 (4.2) | 15.5 (1.3) | 0.645 |
| | 0.3 | 13.2 (5.1) | 11.8 (6.1) | 0.959 |
| V-1 | 30 | 21.8 (4.1) | 15.9 (2.5) | 0.004 |
| | 3 | 19.9 (3.6) | 14.8 (3.9) | 0.017 |
| | 0.3 | 16.0 (3.3) | 13.0 (6.5) | 0.272 |
| V-35 | 30 | 19.4 (5.5) | 18.4 (2.0) | 0.672 |
| | 3 | 20.0 (7.8) | 19.7 (2.3) | 1.000 |
| | 0.3 | 12.0 (7.7) | 14.8 (5.1) | 0.392 |
| V-34 | 30 | 18.6 (3.2) | 14.8 (2.2) | 0.015 |
| | 3 | 19.2 (6.2) | 19.3 (3.6) | 0.878 |
| | 0.3 | 12.0 (5.4) | 15.7 (4.5) | 0.155 |
| V-36 | 30 | 16.7 (4.1) | 10.3 (5.3) | 0.016 |
| | 3 | 17.8 (6.0) | 12.8 (3.5) | 0.083 |
| | 0.3 | 11.6 (9.2) | 8.6 (5.3) | 0.443 |
| IV-9 | 30 | 15.2 (2.5) | 9.4 (2.8) | <0.001 |
| | 3 | 15.6 (3.3) | 9.1 (4.7) | 0.006 |
| | 0.3 | 12.8 (3.3) | 6.0 (7.9) | 0.040 |
| IV-62 | 30 | 13.0 (4.7) | 8.3 (5.5) | 0.088 |
| | 3 | 9.7 (4.6) | 4.0 (9.1) | 0.382 |
| | 0.3 | 5.4 (4.6) | 2.4 (9.1) | 0.574 |
| IV-60 | 30 | 5.2 (6.9) | 7.8 (3.4) | 0.721 |
| | 3 | 7.8 (5.9) | 4.0 (2.9) | 0.234 |
| | 0.3 | 5.2 (6.6) | 5.5 (5.5) | 0.920 |
| IV-8 | 30 | 0.2 (11.3) | 0.7 (6.0) | 0.721 |
| | 3 | 11.4 (2.3) | −1.0 (3.7) | <0.001 |
| | 0.3 | 5.1 (4.8) | 0.7 (6.4) | 0.141 |
| IV-59 | 30 | −1.3 (4.4) | −0.4 (4.1) | 0.688 |
| | 3 | 8.4 (4.7) | 3.9 (2.1) | 0.083 |
| | 0.3 | 4.4 (4.1) | 0.2 (6.6) | 0.130 |

A number of embodiments of the invention have been described. Nevertheless, it will be understood that various modifications may be made without departing from the spirit and scope of the invention. Accordingly, other embodiments are within the scope of the following claims.

1. A method of treating or ameliorating one or more symptoms associated with a protein synthesis inactivating toxin poisoning in a subject, the method comprising administering to the subject a compound according to Formula I-A: 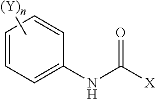
wherein:
X is selected from C₁₋₁₀ alkyl, C₅₋₁₂ cycloalkyl, C₅₋₁₂ aryl, or C₅₋₁₂ heteroaryl, wherein the alkyl, cycloalkyl, aryl, or heteroaryl may be substituted with one or more of C₁₋₁₀ alkyl, OR¹, NO₂, CONR¹R², COR¹, and halo;
each Y is independently H, C₁₋₁₀ alkyl, CO₂R¹, OR¹, or halo;
R¹ and R² are independently H, C₁₋₁₀ alkyl, and aryl; and
n is 1, 2, or 3; or
a pharmaceutically acceptable salt or derivative thereof.

2-6. (canceled)

7. A method of treating or ameliorating one or more symptoms associated with a protein synthesis inactivating toxin poisoning in a subject, the method comprising administering to the subject a compound according to Formula II-A: 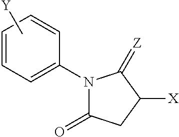
wherein:
X is selected from CO₂R¹, NR¹R², or C₅₋₁₂ heterocycloalkyl;
Y is selected from H, C₁₋₁₀ alkyl, OR¹, or halo;
Z is absent or O;
R¹ is H or C₁₋₁₀ alkyl; and
R² is selected from H, C₁₋₁₀ alkyl; and C₅₋₁₂ cycloalkyl, wherein the alkyl and cycloalkyl may be substituted with C₁₋₁₀ alkyl or C₅₋₁₂ heterocycloalkyl, wherein the heterocycloalkyl may be substituted with a C₁₋₁₀ alkyl; or
a pharmaceutically acceptable salt or derivative thereof.

8. (canceled)

9. A method of treating or ameliorating one or more symptoms associated with a protein synthesis inactivating toxin poisoning in a subject, the method comprising administering to the subject a compound according to Formula III-A: 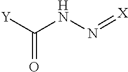
wherein:
X is C₁₋₁₀ alkyl, C₅₋₁₂ cycloalkyl, or C₅₋₁₂ heteroalkyl, wherein the alkyl and heteroaryl can be substituted with one or more of CO₂R¹, OR¹, and halo;
Y is selected from C₅₋₁₂ aryl, C₅₋₁₂ cycloalkyl, and C₅₋₁₂ heterocycle, wherein the heterocycle can be substituted with one or more of OR¹ and NR¹R²; and
R¹ and R² are independently selected from H and C₁₋₁₀ alkyl; or
a pharmaceutically acceptable salt or derivative thereof.

10. (canceled)

11. A method of treating or ameliorating one or more symptoms associated with a protein synthesis inactivating toxin poisoning in a subject, the method comprising administering to the subject a compound according to Formula IV-A: 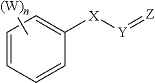
each W is independently C₁₋₁₀ alkyl, CO₂R¹, OR¹, or halo;
X is absent or NH;
Y is N or CH;
Z is selected from C₁₋₁₀ alkyl, C₁₋₁₀ alkenyl, C₁₋₁₀ aralkyl, C₁₋₁₀ heteroaralkyl, C₅₋₁₂ cycloalkyl, and C₅₋₁₂ heterocycle, wherein the alkyl, aralkyl, heteroaralkyl, and heterocycle can be substituted with one or more of C₁₋₁₀ alkyl, C(NH)NH₂, NR¹R², (CH₂)ₘNR¹R², OR¹, (CH₂)ₘOR¹, CN, NO₂, COR¹, CO₂R¹, CF₃, OCF₃, SO₃H, halo, and ═O;
R¹ and R² are independently selected from H, COCH₃, C₁₋₁₀ alkyl, (CH₂)ₘOH, and C₁₋₁₀ aryl;
m is an integer from one to three; and
n is an integer from one to three; or
a pharmaceutically acceptable salt or derivative thereof.

12. (canceled)

13. A method of treating or ameliorating one or more symptoms associated with a protein synthesis inactivating toxin poisoning in a subject, the method comprising administering to the subject a compound according to Formula V-A: 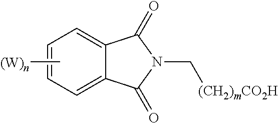
wherein
each W is independently C₁₋₁₀ alkyl, C₂₋₁₀ alkenyl, C₂₋₁₀ alkynyl, CO₂R¹, OR¹, halo, NO₂, NR¹R², or two W come together to form a fused aryl, heteroaryl, cycloalkyl, or heterocycloalkyl, wherein the alkyl, alkenyl or alkynyl can be unsubstituted or substituted with CO₂R¹, OR¹, or halo;
R¹ and R² are independently selected from H and C₁₋₁₀ alkyl;
m is an integer from zero to five; and
n is an integer from zero to three;
or a pharmaceutically acceptable salt or derivative thereof.

14. (canceled)

15. A method of treating or ameliorating one or more symptoms associated with a protein synthesis inactivating toxin poisoning in a subject, the method comprising administering to the subject a compound according to Formula VI-A:
A-(CH₂)ₙ-B
wherein
A is selected from the group consisting of: 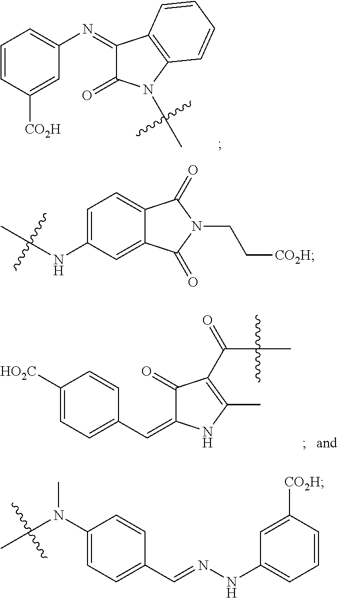
B is selected from the group consisting of: 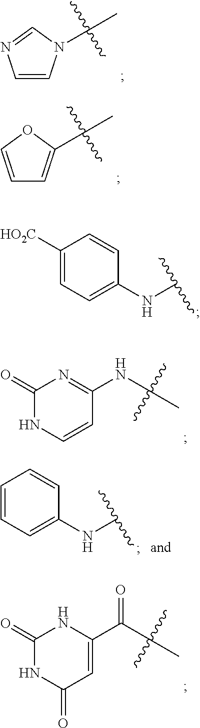
and
n is an integer from four to ten;
or a pharmaceutically acceptable salt or derivative thereof.

16. (canceled)

17. A method of treating or ameliorating one or more symptoms associated with a protein synthesis inactivating toxin poisoning in a subject, the method comprising administering to the subject a compound selected from: 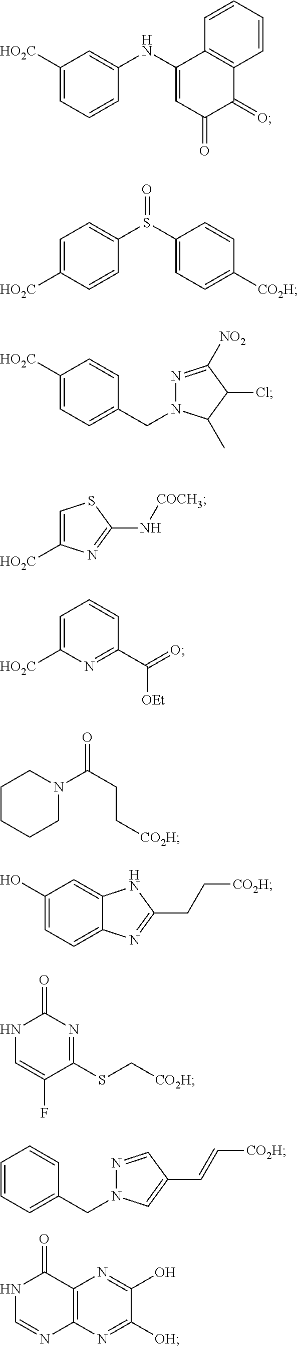 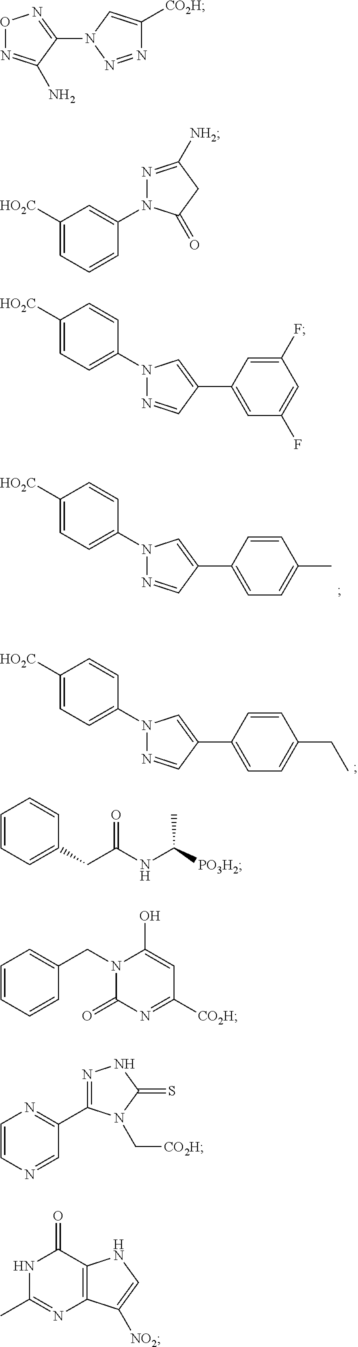 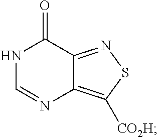
or a pharmaceutically acceptable salt or derivative thereof.

18. A method of treating or ameliorating one or more symptoms associated with a protein synthesis inactivating toxin poisoning in a subject, the method comprising administering to the subject a compound having the structure: 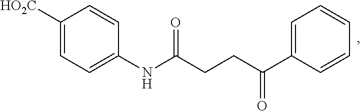
or pharmaceutically acceptable salt or derivative thereof.

19. A method of treating or ameliorating one or more symptoms associated with a protein synthesis inactivating toxin poisoning in a subject, the method comprising administering to the subject a compound having the structure: 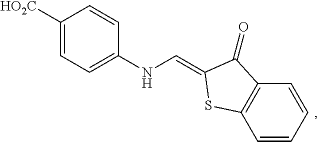
or pharmaceutically acceptable salt or derivative thereof.

20. A method of treating or ameliorating one or more symptoms associated with a protein synthesis inactivating toxin poisoning in a subject, the method comprising administering to the subject a compound having the structure: 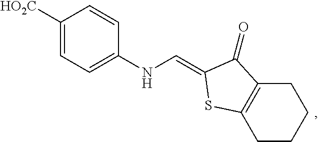
or pharmaceutically acceptable salt or derivative thereof.

21. A method of treating or ameliorating one or more symptoms associated with a protein synthesis inactivating toxin poisoning in a subject, the method comprising administering to the subject a compound having the structure: 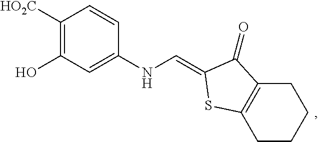
or pharmaceutically acceptable salt or derivative thereof.

22. A method of treating or ameliorating one or more symptoms associated with a protein synthesis inactivating toxin poisoning in a subject, the method comprising administering to the subject a compound having the structure: 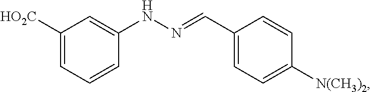
or pharmaceutically acceptable salt or derivative thereof.

23. A method of treating or ameliorating one or more symptoms associated with a protein synthesis inactivating toxin poisoning in a subject, the method comprising administering to the subject a compound having the structure: 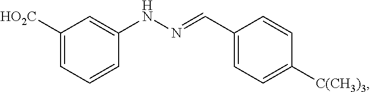
or pharmaceutically acceptable salt or derivative thereof.

24. A method of treating or ameliorating one or more symptoms associated with a protein synthesis inactivating toxin poisoning in a subject, the method comprising administering to the subject a compound having the structure: 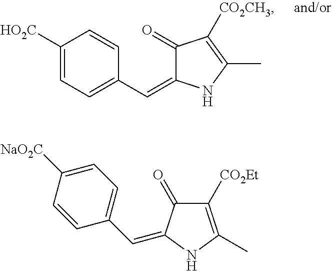
or pharmaceutically acceptable salt or derivative thereof.

25. A method of treating or ameliorating one or more symptoms associated with a protein synthesis inactivating toxin poisoning in a subject, the method comprising administering to the subject a compound having the structure: 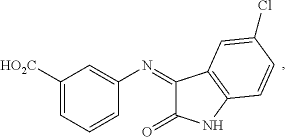
or pharmaceutically acceptable salt or derivative thereof.

26. A method of treating or ameliorating one or more symptoms associated with a protein synthesis inactivating toxin poisoning in a subject, the method comprising administering to the subject a compound having the structure: 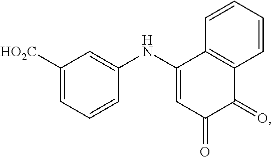
or pharmaceutically acceptable salt or derivative thereof.

27. A method of treating or ameliorating one or more symptoms associated with a protein synthesis inactivating toxin poisoning in a subject, the method comprising administering to the subject a compound having the structure: 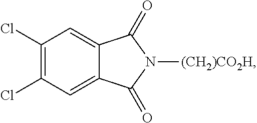
or pharmaceutically acceptable salt derivative thereof.

28. The method of any one of claims 1, 7, 9, 11, 13, 15, or 17-27, wherein the protein synthesis inactivating toxin is selected from: a ribonuclease, an N-glycosidase, and an ADP-ribosyltransferase.

29.-71. (canceled)

72. A pharmaceutical composition comprising a compound of any one of claims 1, 7, 9, 11, 13, 15, or 17-27 and a pharmaceutically acceptable carrier, excipient, or adjuvant.

73. A method of inhibiting type II ribosome inactivating protein poisoning in a subject, the method comprising administering to the subject any compound of claims 1, 7, 9, 11, 13, 15, or 17-27 in combination with a type II ribosome inactivating protein vaccine.

74. (canceled)

75. (canceled)

76. A computer-assisted method of generating a test inhibitor of the active site of ricin, the method using a programmed computer comprising a processor and an input device, the method comprising:
(a) inputting on the input device data comprising a docking box surrounded by one or more amino acid residues of the active site of ricin, the residues having a confirmation as set forth in crystal structure PDB code 1IFS;
(b) docking into the docking box a test inhibitor molecule using the processor; and
(c) determining, based on the docking, whether the test inhibitor molecule would be capable of interacting with one or more residues of the ricin active site.

77.-86. (canceled)

87. A computer-assisted method of generating a test inhibitor of the active site of ricin, the method using a computing device, the method comprising:
(a) receiving on a computing device data comprising a docking box surrounded by one or more amino acid residues of the active site of ricin, the residues having a confirmation as set forth in crystal structure PDB code 1IFS;
(b) docking into the docking box a test inhibitor molecule using the computing device; and
(c) determining, using the computing device, based on the docking, whether the test inhibitor molecule would be capable of interacting with one or more residues of the ricin active site.
